(19) United States
(12) Patent Application Publication
(10) Pub. No.: US 2026/0125356 A1
(43) Pub. Date: May 7, 2026

(54) TREATMENT OF LEISHMANIASIS

(71) Applicant: THE UNIVERSITY OF DURHAM, Durham (GB)

(72) Inventors: Paul DENNY, Durham (GB); Patrick STEEL, Durham (GB); Vanessa LYNE, Durham (GB)

(21) Appl. No.: 19/122,141

(22) PCT Filed: Oct. 13, 2023
(86) PCT No.: PCT/GB2023/052661

§ 371 Date: Apr. 17, 2025

(30) Foreign Application Priority Data
Oct. 17, 2022 (GB) 2215290.4

(51) Int. Cl. C07D 333/44; A61K 9/127; A61K 31/381; A61K 31/4709; A61K 31/536; A61K 31/5377; A61P 31/04; A61P 33/02; C07D 409/06; C07D 409/12; C07D 413/04

(52) U.S. Cl. CPC C07D 333/44; A61K 9/127; A61K 31/381; A61K 31/4709; A61K 31/536; A61K 31/5377; A61P 31/04; A61P 33/02; C07D 409/06; C07D 409/12; C07D 413/04

(57) ABSTRACT

The presentation invention relates to compounds of formula (I) and pharmaceutical compositions thereof. The compounds may be used to treat an infection, such as a parasitic infection or a bacterial infection. In some embodiments, the compounds may be used to treat leishmaniasis.

The invention relates to compounds of formula (I), which may be used in the treatment of microbial infections. In particular, compounds of formula (I) are effective at treating leishmaniasis. The invention extends to novel compositions, therapies and methods for treating, preventing or ameliorating a microbial infection.

The Neglected Tropical Disease (NTD) leishmaniasis is endemic in over 90 countries worldwide, affecting approximately 12 million people per year with 350 million people living at risk of disease. The causative agent, Leishmania species, are sand fly borne kinetoplastid protozoan parasites and infection leads to a wide spectrum of clinical manifestations in endemic areas, from self-healing but scarring cutaneous leishmaniasis (CL) to fatal visceral disease (VL). Largely due to elimination efforts in south Asia, the global burden of VL has decreased substantially in the past decade.

However, due to forced migration, the cases of CL have substantially increased in the same period (0.7-1 million per year). Current treatment of CL largely relies on the pentavalent antimonials such as sodium stibogluconate (Pentostam) and meglumine antimoniate (Glucantime) which have been in clinical use for over 70 years despite their associated problems, which include severe side-effects such as cardiotoxicity and the fact that they require parenteral administration.

Animals can also be infected and serve as reservoirs of disease. In particular, the disease affects dogs throughout southern Europe, South America and the southern USA. Furthermore, the owners of infected companion animals seek their treatment and veterinary drugs are extremely limited in both number and efficacy.

Accordingly, there is a recognised need to develop new and effective therapies for this NTD. The present invention arose from the inventors' work in attempting to address this problem.

In accordance with a first aspect of the invention, there is provided a compound of formula (I):

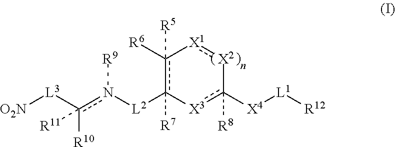

wherein X¹ is CR¹, C(R¹)₂, N, NR¹, O or S;
X² is CR², C(R²)₂, N, NR², O or S;
X³ is CR³, C(R³)₂, N, NR³, O or S;
X⁴ is a bond or NR⁴, O or S;
L¹ is optionally substituted C₁₋₁₂ alkylene, optionally substituted C₂₋₁₂ alkenylene, optionally substituted C₂₋₁₂ alkynylene or -(L⁴O)ₘL⁵-;
L¹ is a bond or is optionally substituted C₁₋₁₂ alkylene, optionally substituted C₂₋₁₂ alkenylene, optionally substituted C₂₋₁₂ alkynylene or -(L₄O)ₘL⁵-;
L³ is an optionally substituted 5 to 10 membered heteroarylene or an optionally substituted C₆-10 arylene;
L⁴ is optionally substituted C₁₋₆ alkylene, optionally substituted C₂₋₆ alkenylene or optionally substituted C₂₋₆ alkynylene;
L⁵ is a bond or optionally substituted C₁₋₆ alkylene, optionally substituted C₂₋₆ alkenylene or optionally substituted C₂₋₆ alkynylene;
R¹, R² and R³ are each independently H, optionally substituted C₁₋₁₂ alkyl, optionally substituted C₂₋₁₂ alkynyl, optionally substituted C₂₋₁₂ alkenyl, OR¹⁸, SR¹³, NR¹³R¹⁴, COR¹³, COOR¹³, CONR¹³R¹⁴, CN or a halogen;
R⁴ is H, optionally substituted C₁₋₁₂ alkyl, optionally substituted C₂₋₁₂ alkenyl or optionally substituted C₂₋₁₂ alkynyl;
R⁵, R⁷ and R⁸ are independently absent or H, optionally substituted C₁₋₁₂ alkyl, optionally substituted C₂₋₁₂ alkynyl, optionally substituted C₂₋₁₂ alkenyl, OR¹³, SR¹³, NR¹³R¹⁴, COR¹³, COOR¹³, CONR¹³R¹⁴, CN or a halogen;
R⁶ is H, optionally substituted C₁₋₁₂ alkyl, optionally substituted C₂₋₁₂ alkynyl, optionally substituted C₂₋₁₂ alkenyl, OR¹³, SR¹³, NR¹³R¹⁴, COR¹³, COOR¹³, CONR¹³R¹⁴, CN or a halogen; R⁹ is absent or is H, optionally substituted C₁₋₁₂ alkyl, optionally substituted C₂₋₁₂ alkenyl or optionally substituted C₂₋₁₂ alkynyl; or R⁶ and R⁹ together with the atoms to which they are attached combine to form an optionally substituted 5 or 6 membered heterocycle or an optionally substituted 5 or 6 membered heteroaryl;
R¹⁰ is H, halogen, optionally substituted C₁₋₁₂ alkyl, optionally substituted C₂₋₁₂ alkenyl or optionally substituted C₂₋₁₂ alkenyl; R¹¹ is absent or H, halogen, optionally substituted C₁₋₁₂ alkyl, optionally substituted C₂₋₁₂ alkenyl or optionally substituted C₂₋₁₂ alkenyl; or R⁶ and R¹⁰ together with the atoms to which they are attached combine to form an optionally substituted 5 or 6 membered heterocycle or an optionally substituted 5 or 6 membered heteroaryl; or R¹⁰ and R¹¹ together form an oxo group;
R¹² is NR¹³R¹⁴, an optionally substituted 5 to 10 membered heteroaryl or an optionally substituted 3 to 10 membered heterocycle, where the heteroaryl or heterocycle contain at least one nitrogen;
R¹³ and R¹⁴ are independently H, optionally substituted C₁₋₁₂ alkyl, optionally substituted C₂₋₁₂ alkenyl or optionally substituted C₂₋₁₂ alkynyl;
n is 0 or 1; and
m is an integer between 1 and 5;
or a pharmaceutically acceptable complex, salt, solvate, tautomeric form or polymorphic form thereof.

Advantageously, the inventors have found that compounds of formula (I) can be used to treat leishmaniasis.

The term “alkyl”, as used herein, unless otherwise specified, refers to a saturated straight or branched hydrocarbon. In certain embodiments, the alkyl group is a primary, secondary, or tertiary hydrocarbon. In certain embodiments, the alkyl group includes one to six carbon atoms, i.e. C₁-C₆ alkyl, C₁-C₆ alkyl includes for example methyl, ethyl, n-propyl (1-propyl) and isopropyl (2-propyl, 1-methylethyl), butyl, pentyl, hexyl, isobutyl, sec-butyl, tert-butyl, isopentyl, neopentyl, and isohexyl. An alkyl group can be unsubstituted or substituted with one or more of halogen, OR¹³, SR¹³, NR¹³R¹⁴, CONR¹³R¹⁴, CN, COR¹³ and COOR¹³. Accordingly, it will be appreciated that an optionally substituted C₁-C₆ alkyl may be an optionally substituted C₁-C₆ haloalkyl, i.e. a C₁-C₆ alkyl substituted with at least one halogen, and optionally further substituted with one or more of OR¹³, SR¹³, NR¹³R¹⁴, CONR¹³R¹⁴, CN, COR¹³ and COOR¹³.

“Alkenyl” refers to olefinically unsaturated hydrocarbon groups which can be unbranched or branched. In certain embodiments, the alkenyl group has 2 to 6 carbons, i.e. it is a C₂-C₆ alkenyl. C₂-C₆ alkenyl includes for example vinyl, allyl, propenyl, butenyl, pentenyl and hexenyl. An alkenyl group can be unsubstituted or substituted with one or more of optionally substituted C₁-C₆ alkyl, optionally substituted C₂₋₆ alkynyl, halogen, OR¹³, SR¹³, NR¹³R¹⁴, CONR¹³R¹⁴, CN, COR¹³ and COOR¹³.

“Alkynyl” refers to acetylenically unsaturated hydrocarbon groups which can be unbranched or branched. In certain embodiments, the alkynyl group has 2 to 6 carbons, i.e. it is a C₂-C₆ alkynyl. C₂-C₆ alkynyl includes for example propargyl, propynyl, butynyl, pentynyl and hexynyl. An alkynyl group can be unsubstituted or substituted with one or more of optionally substituted C₁-C₆ alkyl, optionally substituted C₂₋₆ alkenyl, halogen, OR¹³, SR¹³, NR¹³R¹⁴, CONR¹³R¹⁴, CN, COR¹³ and COOR¹³.

The term “alkylene”, as used herein, unless otherwise specified, refers to a bivalent saturated straight or branched hydrocarbon. “Alkenylene” refers to a bivalent olefinically unsaturated hydrocarbon group which can be unbranched or branched. “Alkynylene” refers to a bivalent acetylenically unsaturated hydrocarbon group which can be unbranched or branched. An optionally substituted alkylene, alkenylene or alkynylene group may be the same as an optionally substituted alkyl, alkenyl or alkynyl group, respectively, as defined above, except the optionally substituted alkylene, alkenylene or alkynylene group is bivalent.

“Arylene” refers to a bivalent aromatic 6 to 10 membered hydrocarbon group. Examples of a C₆-C₁₀ aryl group include, but are not limited to, phenylene, α-naphthylene, β-naphthylene, tetrahydronaphthylene and indanylene. An arylene group can be unsubstituted or substituted with one or more of optionally substituted C₁-C₆ alkyl, optionally substituted C₂₋₆ alkenyl, optionally substituted C₂₋₆ alkynyl, halogen, OR¹³, SR¹³, NR¹³R¹⁴, CONR¹³R¹⁴, CN, COR¹³ and COOR¹³.

“Heteroaryl” refers to a monocyclic or bicyclic aromatic 5 to 10 membered ring system in which at least one ring atom is a heteroatom. The or each heteroatom may be independently selected from the group consisting of oxygen, sulfur and nitrogen. Examples of 5 to 10 membered heteroaryl groups include furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, and isoquinoline. Bicyclic 5 to 10 membered heteroaryl groups include those where a phenyl, pyridine, pyrimidine, pyrazine or pyridazine ring is fused to a 5 or 6-membered monocyclic heteroaryl ring. A heteroaryl group can be unsubstituted or substituted with one or more of optionally substituted C₁-C₆ alkyl, optionally substituted C₂₋₆ alkenyl, optionally substituted C₂₋₆ alkynyl, halogen, OR¹³, SR¹³, NR¹³R¹⁴, CONR¹³R¹⁴, CN, COR¹³ and COOR¹³.

The term “heteroarylene”, as used herein, unless otherwise specified, refers to a bivalent monocyclic or bicyclic aromatic 5 to 10 membered ring system in which at least one ring atom is a heteroatom. An optionally substituted heteroarylene group may be the same as an optionally substituted heteroaryl group, as defined above, except the optionally substituted heteroaryl group is bivalent.

“Heterocycle” or “heterocyclyl” refers to a 3 to 10 membered monocyclic, bicyclic or bridged molecules in which at least one ring atom is a heteroatom. The or each heteroatom may be independently selected from the group consisting of oxygen, sulfur and nitrogen. A heterocycle may be saturated or partially saturated. Exemplary heterocyclyl groups include but are not limited to aziridine, oxirane, oxirene, thiirane, pyrroline, pyrrolidine, dihydrofuran, tetrahydrofuran, dihydrothiophene, tetrahydrothiophene, dithiolane, piperidine, 1,2,3,6-tetrahydropyridine-1-yl, tetrahydropyran, pyran, morpholine, piperazine, thiane, thiine, piperazine, azepane, diazepane, oxazine. A heterocyclyl group can be unsubstituted or substituted with one or more of optionally substituted C₁-C₆ alkyl, optionally substituted C₂₋₆ alkenyl, optionally substituted C₂₋₆ alkynyl, halogen, OR¹³, SR¹³, NR¹³R¹⁴, CONR¹³R¹⁴, CN, COR¹³ and COOR¹³.

In some embodiments, n may be 0. Accordingly, the compound of formula (I) may be a compound of formula (Ia):

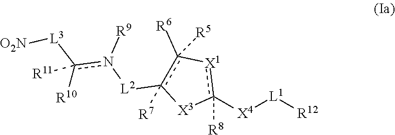

In the compound of formula (I) or (Ia), X¹ may be CR¹ or C(R¹) ₂.

In one embodiment, X¹ is CR¹ and R⁵, R⁷ and R⁸ are absent.

In the compound of formula (I) or (Ia), X³ may be CR³, NR³, O or S. In some embodiments, X³ may be CR³ or S.

Accordingly, the compound of formula (Ia) may be a compound of formula (Iai), (Iaii) or (Iaiii):

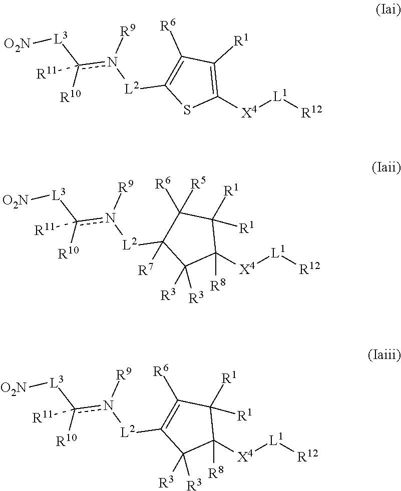

In alternative embodiments, n may be 1. Preferably, X¹ is CR¹ or N, X² is CR² or N, X³ is CR³ or N, and R⁵, R⁷ and R⁸ are absent. Accordingly, the compound of formula (I) may be a compound of formula (Ib):

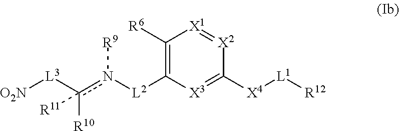

In the compound of formula (I) or (Ib), X¹ may be CR¹ or N. Preferably, X¹ is CR¹.

In the compound of formula (I) or (Ib), X² may be CR².

In the compound of formula (I) or (Ib), X³ may be CR³.

Accordingly, the compound of formula (Ib) may be a compound of formula (Ibi):

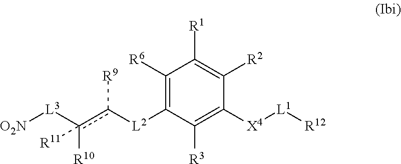

In the compound of formula (I), or any of the embodiments described above, preferably at least one of R¹, R², R³, R⁵, R⁶, R⁷ and R⁸ is optionally substituted C₁₋₁₂ alkyl, optionally substituted C₂₋₁₂ alkynyl, optionally substituted C₂₋₁₂ alkenyl, OR¹³, SR¹³, NR¹³R¹⁴, COR¹³, COOR¹³, CONR¹³R¹⁴, CN or a halogen. More preferably, at least one of R¹, R², R³, R⁵, R⁶, R⁷ and R⁸ is optionally substituted C₁₋₆ alkyl, optionally substituted C₂₋₆ alkynyl, optionally substituted C₂₋₆ alkenyl, OR¹³, SR¹³, NR¹³R¹⁴, COR¹³, COOR¹³, CONR¹³R¹⁴ or CN. More preferably, at least one of R¹, R², R³, R⁵, R⁶, R⁷ and R⁸ is optionally substituted C₁₋₃ alkyl, optionally substituted C₂₋₃ alkynyl, optionally substituted C₂₋₃ alkenyl, OR¹³, SR¹³, NR¹³R¹⁴, COR¹³, COOR¹³, CONR¹³R¹⁴ or CN. Even more preferably, at least one of R¹, R², R³, R⁵, R⁶, R⁷ and R⁸ is optionally substituted methyl, OR¹³, SR¹³, NR¹³R¹⁴, COR¹³, COOR¹³, CONR¹³R¹⁴ or CN. Even more preferably, one of R¹, R², R³, R⁵, R⁶, R⁷ and R⁸ is COOR¹³ or CONR¹³R¹⁴. Most preferably, one of R¹, R², R³, R⁵, R⁶, R⁷ and R⁸ is CONR¹³R¹⁴. R¹³ and R¹⁴ may independently be H, optionally substituted C16 alkyl, optionally substituted C₂₋₆ alkenyl or optionally substituted C₂₋₆ alkynyl. Preferably, R¹³ and R¹⁴ are independently H, optionally substituted C₁₋₃ alkyl, optionally substituted C₂₋₃ alkenyl or optionally substituted C₂₋₃ alkynyl. More preferably, R¹³ and R¹⁴ are independently H or optionally substituted methyl. In some embodiments, R¹³ is methyl. In some embodiment, R¹⁴ is H. The alkyl, alkenyl and/or alkynyl may be unsubstituted or substituted with one or more halogens. The halogen may be fluorine, chlorine or bromine.

Preferably, at least one of R¹ and R⁶ is optionally substituted C₁₋₁₂ alkyl, optionally substituted C₂₋₁₂ alkynyl, optionally substituted C₂₋₁₂ alkenyl, OR¹⁹, SR¹³, NR¹³R¹⁴, COR¹³, COOR¹³, CONR¹³R¹⁴, CN or a halogen. More preferably, at least one of R¹ and R⁶ is optionally substituted C₁₋₆ alkyl, optionally substituted C₂₋₆ alkynyl, optionally substituted C₂₋₆ alkenyl, OR¹³, SR¹³, NR¹³R¹⁴, COR¹³, COOR¹³, CONR¹³R¹⁴ or CN. More preferably, at least one of R¹ and R⁶ is optionally substituted C₁₋₃ alkyl, optionally substituted C₂₋₃ alkynyl, optionally substituted C₂₋₃ alkenyl, OR¹³, SR¹³, NR¹³R¹⁴, COR¹³, COOR¹³, CONR¹³R¹⁴ or CN. Even more preferably, at least one of R¹ and R⁶ is optionally substituted methyl, OR¹³, SR¹³, NR¹³R¹⁴, COR¹³, COOR¹³, CONR¹³R¹⁴ or CN. Even more preferably, one of R¹ and R⁶ is COOR¹³ or CONR¹³R¹⁴. Even more preferably, one of R¹ and R⁶ is CONR¹³R¹⁴. Most preferably, R⁶ is CONR¹³R¹⁴. R¹³ and R¹⁴ may independently be H, optionally substituted C₁₋₆ alkyl, optionally substituted C₂₋₆ alkenyl or optionally substituted C₂₋₆ alkynyl. Preferably, R¹³ and R¹⁴ are independently H, optionally substituted C₁₋₃ alkyl, optionally substituted C₂₋₃ alkenyl or optionally substituted C₂₋₃ alkynyl. More preferably, R¹³ and R¹⁴ are H or optionally substituted methyl. In some embodiments, R¹³ is methyl. In some embodiment, R¹⁴ is H. The alkyl, alkenyl and/or alkynyl may be unsubstituted or substituted with one or more halogens. The halogen may be fluorine, chlorine or bromine. Accordingly, in a most preferred embodiment, R⁶ is CONHMe.

In some embodiments, R⁶ and R⁹ together with the atoms to which they are attached combine to form an optionally substituted 5 or 6 membered heterocycle or an optionally substituted 5 or 6 membered heteroaryl. Preferably, R⁶ and R⁹ together with the atoms to which they are attached combine to form a 6 membered heterocycle. Preferably L² is a bond. Accordingly, the compound of formula (I) may be a compound of formula (Ic):

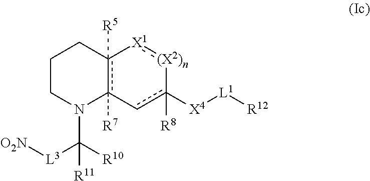

In the compound of formula (Ic), n may be 1. Accordingly, the compound of formula (Ic) may be a compound of formula (Ici) or even more preferably (Icii):

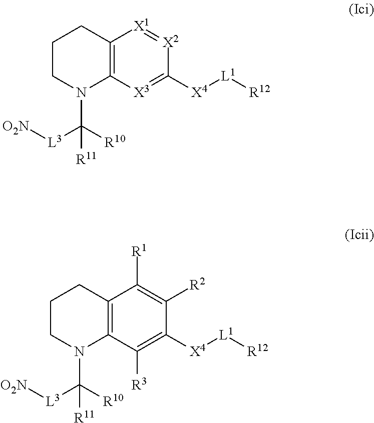

In alternative embodiments, R⁶ and R¹⁰ together with the atoms to which they are attached combine to form an optionally substituted 5 or 6 membered heterocycle or an optionally substituted 5 or 6 membered heteroaryl. Preferably, R⁶ and R¹⁰ together with the atoms to which they are attached combine to form an optionally substituted 6 membered heterocycle. The optionally substituted heterocycle preferably contains a nitrogen and an oxygen in the 5 or 6 membered ring. Preferably, the heterocycle is substituted with an oxo group. Preferably La is a bond. Accordingly, the compound of formula (I) may be a compound of formula (Id):

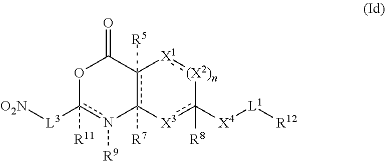

In the compound of formula (Id), n may be 1. Accordingly, the compound of formula (Id) may be a compound of formula (Idi) or even more preferably (Idii):

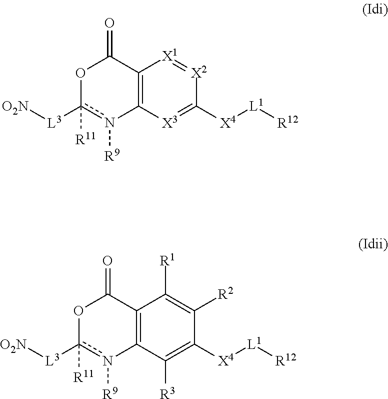

In the compound of formula (Id), (Idi) or (Idii), R⁹ and R¹¹ may be absent. Accordingly, there may be a double bond between the nitrogen and the adjacent carbon in the heterocyclic ring.

Alternatively, or additionally, the remaining R¹, R², R³, R⁵, R⁶, R⁷ and/or R⁸ groups which are not as defined above may be H, optionally substituted C₁₋₁₂ alkyl, optionally substituted C₂₋₁₂ alkynyl, optionally substituted C₂₋₁₂ alkenyl, OR¹³, SR¹³ or a halogen. Preferably, the remaining R¹, R², R³, R⁵, R⁶, R⁷ and/or R⁸ groups which are not as defined above may be H, optionally substituted C₁₋₆ alkyl, optionally substituted C₂₋₆ alkynyl, optionally substituted C₂₋₆ alkenyl, OR¹³, SR¹³ or a halogen. More preferably, the remaining R¹, R², R³, R⁵, R⁶, R⁷ and/or R⁸ groups which are not as defined above may be H, optionally substituted C₁₋₄ alkyl, optionally substituted C₂₋₄ alkynyl, optionally substituted C₂₋₄ alkenyl, OR¹³, SR¹³ or fluorine, chlorine or bromine. More preferably, the remaining R¹, R², R³, R⁵, R⁶, R⁷ and/or R⁸ groups which are not as defined above may be H, optionally substituted methyl, optionally substituted ethyl, optionally substituted propyl, optionally substituted butyl, OR¹³, SR¹³ or bromine. The optionally substituted butyl may be an optionally substituted t-butyl. R¹³ may be H, optionally substituted C₁₋₆ alkyl, optionally substituted C₂₋₆ alkenyl or optionally substituted C₂₋₆ alkynyl. Preferably, R¹³ is H, optionally substituted C₁₋₃ alkyl, optionally substituted C₂₋₃ alkenyl or optionally substituted C₂₋₃ alkynyl. More preferably, R¹³ is H or optionally substituted methyl. In some embodiments, R¹³ is methyl. The alkyl, alkenyl and/or alkynyl may be unsubstituted or substituted with one or more halogens. The halogen may be fluorine, chlorine or bromine. Accordingly, the optionally substituted alkyl may be methyl or CF₃. Most preferably, the remaining R¹, R², R³, R⁵, R⁶, R⁷ and/or R⁸ groups which are not as defined above are H.

In the compound of formula (I), or any of the embodiments described above, X⁴ is preferably O.

In the compound of formula (I), or any of the embodiments described above, L¹ may be optionally substituted C₁₋₆ alkylene, optionally substituted C₂₋₆ alkenylene, optionally substituted C₂₋₆ alkynylene or -(L₄O)ₘL⁵-, and L⁴ and L⁵ may be optionally substituted C₁₋₃ alkylene, optionally substituted C₂₋₃ alkenylene or optionally substituted C₂₋₃ alkynylene and m may be 1, 2 or 3. L¹ may be optionally substituted C₂₋₃ alkylene, optionally substituted C₂₋₃ alkenylene or optionally substituted C₂₋₃ alkynylene. Preferably, L¹ is —CH₂CH₂— or —CH₂CH₂CH₂—.

In the compound of formula (I), or any of the embodiments described above, R¹² may be NR¹³R¹⁴, an optionally substituted 5 or 6 membered heteroaryl or an optionally substituted 5 or 6 membered heterocycle, where the heteroaryl or heterocycle contain at least one nitrogen. R¹³ and R¹⁴ may independently be H, optionally substituted C₁₋₆ alkyl, optionally substituted C₂₋₆ alkenyl or optionally substituted C₂₋₆ alkynyl. More preferably, R¹³ and R¹⁴ are independently H, optionally substituted C₁₋₃ alkyl, optionally substituted C₂₋₃ alkenyl or optionally substituted C₂₋₃ alkynyl. Preferably, R¹² is an optionally substituted 5 or 6 membered heterocycle, where the heterocycle contains at least one nitrogen. The heterocycle may be bonded to the L¹ group through the nitrogen atom. R¹² may be optionally substituted pyrrolidinyl, optionally substituted piperidinyl, optionally substituted piperazinyl, optionally substituted morpholinyl or optionally substituted thiomorpholinyl. The 5 or 6 membered heterocycle may be unsubstituted or substituted with halogen, optionally substituted C₁-C₆ alkyl, optionally substituted C₂₋₆ alkenyl, optionally substituted C₂₋₆ alkynyl or oxo. More preferably, the 5 or 6 membered heterocycle may be unsubstituted or substituted with C₁-C₃ alkyl, C₂₋₃ alkenyl, C₂₋₃ alkynyl or oxo. Most preferably, the 5 or 6 membered heterocycle is unsubstituted or substituted with methyl or oxo. Accordingly, R¹² may be

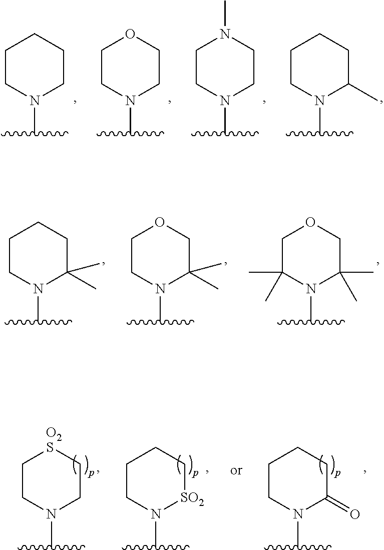

where p is 0 or 1. In one embodiment, R¹² is

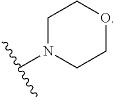 [SMILES: *N1CCOCC1]

In the compound of formula (I), or any of the embodiments described above unless stated otherwise, L² may be a bond or optionally substituted C₁₋₆ alkylene, optionally substituted C₂₋₆ alkenylene or optionally substituted C₂₋₆ alkynylene. Preferably, L² is a bond or optionally substituted C₁₋₃ alkylene, optionally substituted C₂₋₃ alkenylene or optionally substituted C₂₋₃ alkynylene. More preferably, L² is a bond or —CH₂—. Most preferably, L² is a bond.

In the compound of formula (I), or any of the embodiments described above unless stated otherwise, R⁹ and R¹¹ may be absent. It will be appreciated that in this embodiment, there would be a double bond between the nitrogen and the adjacent carbon to which R¹⁰ is attached.

However, in a preferred embodiment of the compound of formula (I), or any of the embodiments described above unless stated otherwise, R⁹ is preferably H, optionally substituted C₁₋₆ alkyl, optionally substituted C₂₋₆ alkenyl or optionally substituted C₂₋₆ alkynyl. More preferably, R⁹ is H, optionally substituted C₁₋₃ alkyl, optionally substituted C₂₋₃ alkenyl or optionally substituted Ca-3 alkynyl. Even more preferably, R⁹ is H or methyl. Most preferably, R⁹ is H.

In the compound of formula (I), or any of the embodiments described above unless stated otherwise, R¹⁰ may be H, halogen, optionally substituted C₁₋₆ alkyl, optionally substituted C₂₋₆ alkenyl or optionally substituted C₂₋₆ alkenyl, R¹¹ may be absent or H, halogen, optionally substituted C₁₋₆ alkyl, optionally substituted C₂₋₆ alkenyl or optionally substituted C₂₋₆ alkenyl or R¹⁰ and R¹¹ may together form an oxo group. More preferably, R¹⁰ may be H, halogen, optionally substituted C₁₋₃ alkyl, optionally substituted C₂₋₃ alkenyl or optionally substituted C₂₋₃ alkenyl, R¹¹ may be absent or H, halogen, optionally substituted C₁₋₃ alkyl, optionally substituted C₂₋₃ alkenyl or optionally substituted C₂₋₃ alkenyl or R¹⁰ and R¹¹ may together form an oxo group. In some embodiments, R¹⁰ may be H and R¹¹ may be absent. However, in a preferred embodiment, R¹⁰ and R¹¹ together form an oxo group.

In the compound of formula (I), or any of the embodiments described above, L³ may be an optionally substituted 5, 6 or 9 membered heteroarylene or an optionally substituted phenylene, more preferably an optionally substituted 5 or 9 membered heteroarylene and most preferably an optionally substituted 5 or 9 membered heteroarylene. Accordingly, L³ may be an optionally substituted 1H-pyrollylene, an optionally substituted furanylene, an optionally substituted thiophenylene, an optionally substituted 1 h-indolylene, an optionally substituted benzofuranylene, an optionally substituted benzthiophenylene or an optionally substituted phenylene. The optionally substituted heteroarylene or optionally substituted aryl may be unsubstituted or substituted with one of more of optionally substituted C₁-C₆ alkyl, optionally substituted C₂₋₆ alkenyl, optionally substituted C₂₋₆ alkynyl, halogen, OR¹³, SR¹³, NR¹³R¹⁴, CONR¹³R¹⁴, CN, COR¹³ and COOR¹³. Preferably, the optionally substituted heteroarylene or optionally substituted aryl is unsubstituted or substituted with one of more of optionally substituted C₁-C₃ alkyl, optionally substituted C₂₋₃ alkenyl, optionally substituted C₂₋₃ alkynyl and halogen. More preferably, the optionally substituted heteroarylene or optionally substituted aryl is unsubstituted or substituted with one of more of optionally substituted methyl and halogen. The optionally substituted alkyl, alkenyl or alkynyl may be unsubstituted or substituted with one or more halogen. The or each halogen may be fluorine, chlorine or bromine and is preferably fluorine. Accordingly, in some embodiments, the optionally substituted heteroarylene or optionally substituted aryl is unsubstituted or substituted with CF₃. Accordingly, L³ may be

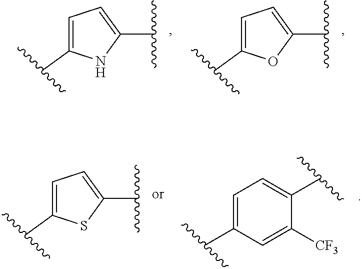 [SMILES: *c1ccc(*)[nH]1 ; *c1ccc(*)o1 ; *c1ccc(*)s1 ; *c1ccc(*)c(C(F)(F)F)c1]

Most preferably, L³ is

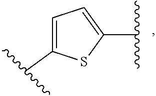 [SMILES: *c1ccc(*)s1]

The compound may be a compound of formula (100) or (101):

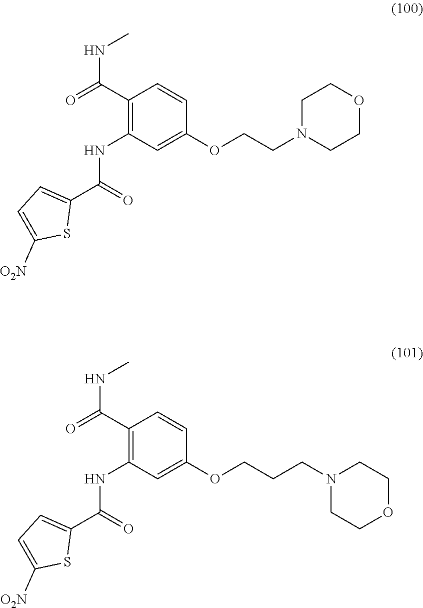 [SMILES: CNC(=O)c1ccc(OCCN2CCOCC2)cc1NC(=O)c1ccc([N+](=O)[O-])s1 ; CNC(=O)c1ccc(OCCCN2CCOCC2)cc1NC(=O)c1ccc([N+](=O)[O-])s1]

In a second aspect, there is provided a pharmaceutical composition comprising a compound of formula (I), or a pharmaceutically acceptable complex, salt, solvate, tautomeric form or polymorphic form thereof, and a pharmaceutically acceptable vehicle.

The pharmaceutical composition can be used in the therapeutic amelioration, prevention or treatment in a subject of a microbial infection.

In accordance with a third aspect, there is provided a compound of formula (I), or a pharmaceutically acceptable complex, salt, solvate, tautomeric form or polymorphic form thereof, or the pharmaceutical composition of the second aspect, for use as a medicament.

In particular, the inventors have found that the compounds of formula (I) may be used to treat a microbial infection.

Accordingly, in accordance with a fourth aspect, there is provided a compound of formula (I), or a pharmaceutically acceptable complex, salt, solvate, tautomeric form or polymorphic form thereof, or the pharmaceutical composition of the second aspect, for use in treating an infection.

According to a fifth aspect of the invention, there is provided a method of treating, preventing or ameliorating an infection in a subject, the method comprising administering to a subject in need of such treatment, a therapeutically effective amount of a compound of formula (I) or a pharmaceutically acceptable complex, salt, solvate, tautomeric form or polymorphic form thereof.

Preferably, the infection is a microbial infection. Preferably, the microbial infection is a parasitic infection. Preferably, the parasitic infection is a protozoan parasitic infection.

The parasitic infection may be leishmaniasis, Chagas disease or African sleeping sickness. Preferably, the parasitic infection is leishmaniasis.

Alternatively, the infection may be a bacterial infection. The bacterial infection may be caused by a gram-positive bacteria or a gram-negative bacteria. The bacterial infection may be caused by a gram-positive bacteria. The bacteria may be from the family Staphylococcus or Escherichia. The bacteria may be S. aureus or E. coli.

The term “preventing” may be understood to mean reducing the likelihood of the patient developing a microbial infection.

Pharmaceutically acceptable salts include any salt of a compound of formula (I) provided herein which retains its biological properties and which is not toxic or otherwise undesirable for pharmaceutical use. The pharmaceutically acceptable salt may be derived from a variety of organic and inorganic counter-ions well known in the art.

The pharmaceutically acceptable salt may comprise an acid addition salt formed with organic or inorganic acids such as hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid and the like acids. Alternatively, the pharmaceutically acceptable salt may comprise a base addition salt formed when an acidic proton present in the parent compound is either replaced by a metal ion, e.g., an alkali metal ion, an alkaline earth ion, an aluminium ion, alkali metal or alkaline earth metal hydroxides, such as sodium, potassium, calcium, magnesium, aluminium, lithium, zinc, and barium hydroxide, or coordinates with an organic base, such as aliphatic, alicyclic, or aromatic organic amines, such as ammonia, methylamine, dimethylamine, diethylamine, picoline, ethanolamine, diethanolamine, triethanolamine, ethylenediamine, lysine, arginine, ornithine, choline, N,N′-dibenzylethylene-diamine, chloroprocaine, diethanolamine, procaine, N-benzylphenethylamine, N-methylglucamine piperazine, tris(hydroxymethyl)-aminomethane, tetramethylammonium hydroxide, and the like.

A pharmaceutically acceptable solvate refers to a compound of formula (I) provided herein, or a salt thereof, that further includes a stoichiometric or non-stoichiometric amount of solvent bound by non-covalent intermolecular forces. Where the solvent is water, the solvate is a hydrate.

It will be appreciated that the compound of formula (I) described herein, or a pharmaceutically acceptable salt or solvate thereof, may be used in a medicament which may be used in a monotherapy (i.e. use of the compound of formula (I) alone), for treating, ameliorating, or preventing a microbial infection. Alternatively, the compound of formula (I) or a pharmaceutically acceptable salt or solvate thereof may be used as an adjunct to, or in combination with, known therapies for treating, ameliorating, or preventing a microbial infection.

The compound of formula (I) may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment.

In some embodiments, the compound of formula (I) is provided in a liposomal suspension or formulation. The liposomal formulation may comprise a plurality of lipids. The plurality of lipids may comprise a phospholipid. The phospholipid may be or comprise phosphatidylcholine. The plurality of lipids may comprise a cationic lipid and/or the ionisable cationic lipid. The cationic lipid and/or the ionisable cationic lipid may be or comprise stearylamine.

The liposomal suspension or formulation may comprise a phospholipid and a cationic lipid and/or ionisable cationic lipid. The weight ratio of the phospholipid to the cationic lipid and/or the ionisable cationic lipid may be between 50:1 and 1:1, between 20:1 and 2.5:1, between 17.5:1 and 5:1, between 15:1 and 8:1 or between 12:1 and 9:1, and may be about 10:1. The molar ratio of the phospholipid to the cationic lipid and/or the ionisable cationic lipid may be between 10:1 and 1:2, between 7.5:1 and 1:1, between 5:1 and 2:1, between 4:1 and 3:1, between 3.75:1 and 3.2:1 or between 3.5:1 and 3.4:1.

The weight ratio of the plurality of lipids to the compound of formula (I) may be between 1,000:1 and 1:1, between 500:1 and 10:1, between 250:1 and 25:1, between 150:1 and 50:1, between 100:1 and 60:1, between 90:1 and 70:1 or between 80:1 and 75:1. The molar ratio of the plurality of lipids to the compound of formula (I) may be between 1,000:1 and 1:1, between 500:1 and 5:1, between 250:1 and 10:1, between 100:1 and 20:1, between 80:1 and 30:1, between 60:1 and 40:1 or between 55:1 and 45:1.

It will be appreciated that the vehicle of medicaments according to the invention should be one which is well-tolerated by the subject to whom it is given.

Medicaments comprising the compound of formula (I) described herein may be used in a number of ways. Compositions comprising the compound of formula (I) of the invention may be administered by inhalation (e.g. intranasally). Compositions may also be formulated for topical use. For instance, creams or ointments may be applied to the skin.

The compound of formula (I) according to the invention may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with the compound of formula (I) used according to the invention is required and which would normally require frequent administration (e.g. at least daily injection).

The compound of formula (I) and compositions according to the invention may be administered to a subject by injection into the blood stream or directly into a site requiring treatment. Injections may be intravenous (bolus or infusion) or subcutaneous (bolus or infusion), or intradermal (bolus or infusion).

In a preferred embodiment, the compound of formula (I) is administered orally. Accordingly, the compound of formula (I) may be contained within a composition that may, for example, be ingested orally in the form of a tablet, capsule or liquid.

It will be appreciated that the amount of the compound of formula (I) that is required is determined by its biological activity and bioavailability, which in turn depends on the mode of administration, the physiochemical properties of the compound of formula (I), and whether it is being used as a monotherapy, or in a combined therapy. The frequency of administration will also be influenced by the half-life of the compound of formula (I) within the subject being treated. Optimal dosages to be administered may be determined by those skilled in the art, and will vary with the particular compound of formula (I) in use, the strength of the pharmaceutical composition, the mode of administration, and the advancement of the microbial infection. Additional factors depending on the particular subject being treated will result in a need to adjust dosages, including subject age, weight, sex, diet, and time of administration.

The compound of formula (I) may be administered during or after onset of the microbial infection to be treated. Daily doses may be given as a single administration. Alternatively, the compound of formula (I) may be given two or more times during a day.

Generally, a daily dose of between 0.01 μg/kg of body weight and 500 mg/kg of body weight of the compound of formula (I) according to the invention may be used for treating, ameliorating, or preventing a microbial infection. More preferably, the daily dose is between 0.01 mg/kg of body weight and 400 mg/kg of body weight, more preferably between 0.1 mg/kg and 200 mg/kg body weight, and most preferably between approximately 1 mg/kg and 100 mg/kg body weight.

Alternatively, a slow release device may be used to provide optimal doses of the compound of formula (I) according to the invention to a patient without the need to administer repeated doses.

Known procedures, such as those conventionally employed by the pharmaceutical industry (e.g. in vivo experimentation, clinical trials, etc.), may be used to form specific formulations comprising the compound of formula (I) according to the invention and precise therapeutic regimes (such as daily doses of the compound of formula (I) and the frequency of administration).

A “subject” may be a vertebrate, mammal, or domestic animal. Hence, the compound of formula (I), compositions and medicaments according to the invention may be used to treat any mammal, for example livestock (e.g. a horse), pets (e.g. a dog), or may be used in other veterinary applications. Most preferably, however, the subject is a human being.

A “therapeutically effective amount” of the compound of formula (I) is any amount which, when administered to a subject, is the amount of drug that is needed to treat the microbial infection.

For example, the therapeutically effective amount of the compound of formula (I) used may be from about 0.01 mg to about 800 mg, and preferably from about 0.01 mg to about 500 mg. It is preferred that the amount of the compound of formula (I) is an amount from about 0.1 mg to about 250 mg, and most preferably from about 0.1 mg to about 20 mg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents (i.e. the compound of formula (I)) according to the invention. In tablets, the active compound of formula (I) may be mixed with a vehicle having the necessary compression properties in suitable proportions and compacted in the shape and size desired. The powders and tablets preferably contain up to 99% of the active compound of formula (I). Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The compound of formula (I) according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and Arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The compound of formula (I) may be prepared as a sterile solid composition that may be dissolved or suspended at the time of administration using sterile water, saline, or other appropriate sterile injectable medium.

The compound of formula (I) and compositions of the invention may be administered in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The compound of formula (I) used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

All of the features described herein (including any accompanying claims, figures and abstract), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

FIG. 1 Crystal structure of original hit 2 showing intramolecular hydrogen bonding resulting in close packing of monomer units in the crystal lattice (P-1);

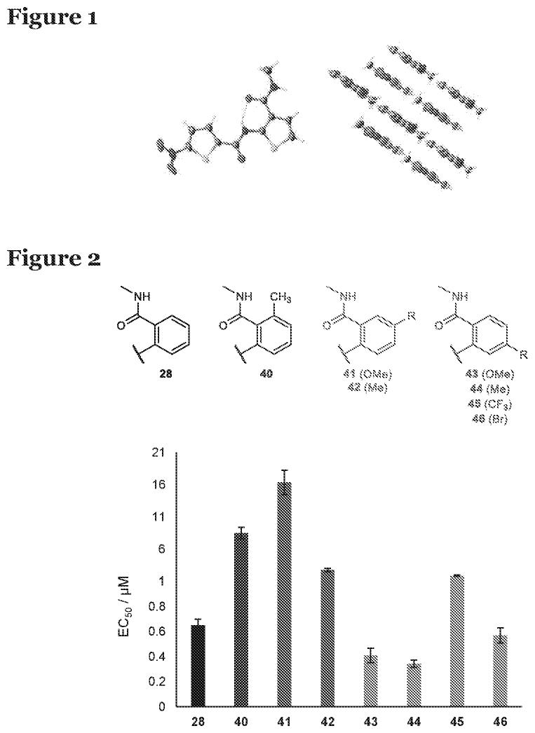

FIG. 2 Antileishmanial activity of substituted phenyl derivatives (40-46) compared to unsubstituted phenyl 28. Averages and standard errors calculated from at least 3 biological repeats;

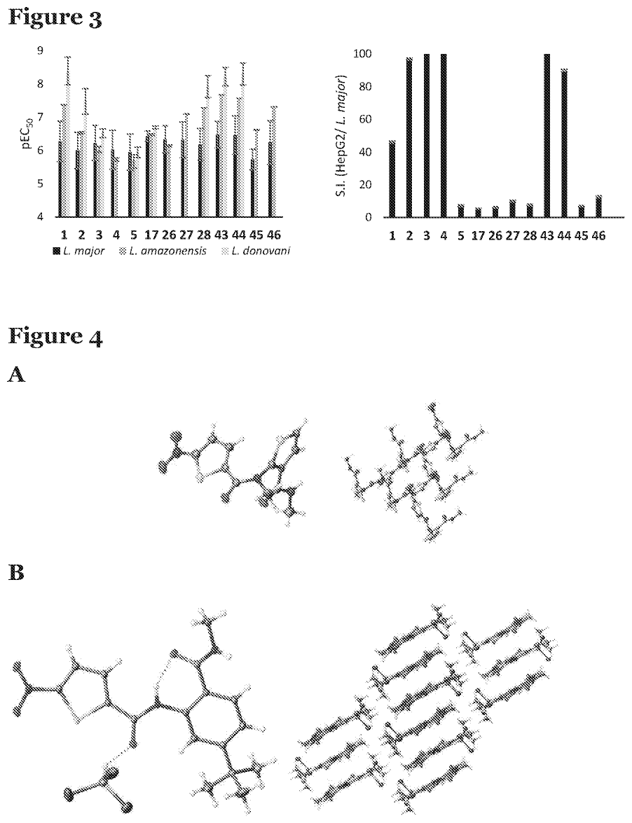

FIG. 3 (A) 13 5N2C compounds show broad spectrum antileishmanial activity. (B) 6 5N2C compounds show a selectivity index over HepG2 cells>30. Averages were calculated from the mean of at least three biological repeats. Errors represent standard error;

FIG. 4 (A) Crystal structures of methyl capped analogue 48 showing loss of intramolecular hydrogen bonding and an alternative folded geometry; (B) Crystal structures of tert-butyl substituted analogue 51;

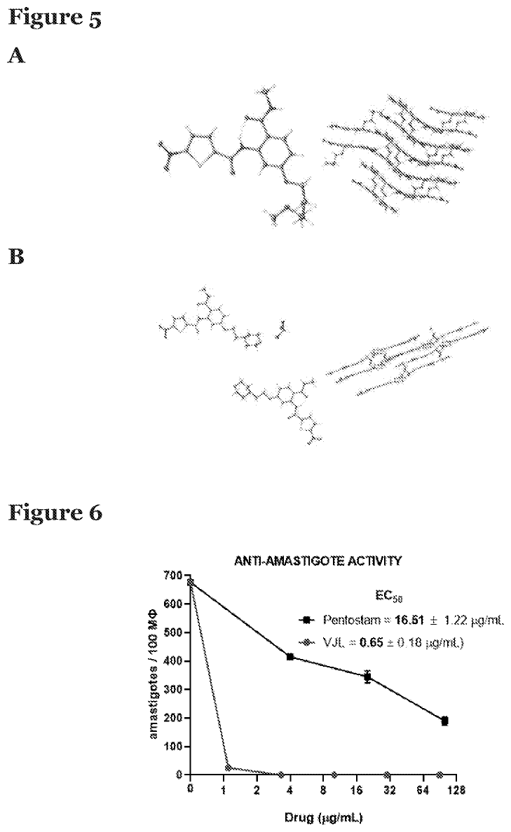

FIG. 5 (A) X-ray crystal structure of 51 showing accommodation of the PEG chain within the crystal lattice. (B) X-ray crystal structure of 52 showing an increase in free space within the lattice to accommodate the morpholine ring;

FIG. 6 shows compound 53 (identified as VJL) killing of intracellular amastigotes. Peritoneal macrophages were plated at 2×10⁵/well in 24 well-plate onto circular glass coverslips with medium RPMI+10% HIFCS. After 24 h, macrophages were infected with L. amazonensis (1:10) and cultured at 37″C for 4 h and washed to remove non internalized amastigotes. At 24 h post infection, cells were treated with serial dilutions of compound 53 or Pentostam for 48 h at 37° C. in medium RPMI+5% HIFCS. Afterwards, cells were stained with Rapid Panoptic and the number of intracellular amastigotes in 100 macrophages was counted. EC₅₀ values were calculated by non-linear regression. Means±SD (n=2);

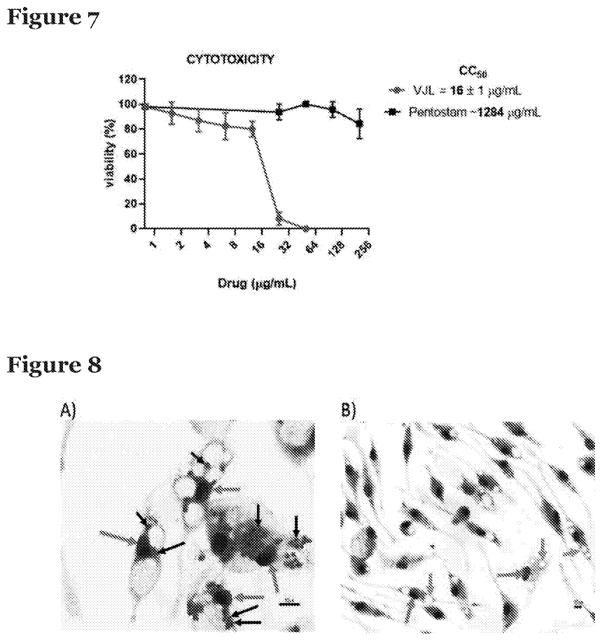

FIG. 7 shows cytotoxicity of compound 53 against macrophages. Mouse bone marrow-derived macrophages (BMDM) were plated in 96 well-plate at 1×10⁵/well with medium RPMI+10% HIFCS. After 24 h, compound 53 and Pentostam were added at the indicated concentrations, and cells cultured for further 48 h with medium RPMI+5% HIFCS. Resazurin solution was added in the last 4 hours, and fluorescence recorded CC₅₀ values were calculated by non-linear regression, where 100% viability were cells cultured in the absence of drugs. Pentostam CC₅₀ was from curve regression extrapolation. Means±SD (n=5);

FIG. 8 shows photographs of L. amazonensis-infected macrophages. A) Untreated, 400×. B) Treated with 1 μg/mL of compound 53, 200×. Bar=10 μm. Red arrows: Macrophage nuclei, Black arrows=amastigotes, Green arrows=empty parasitophorus vacuoles;

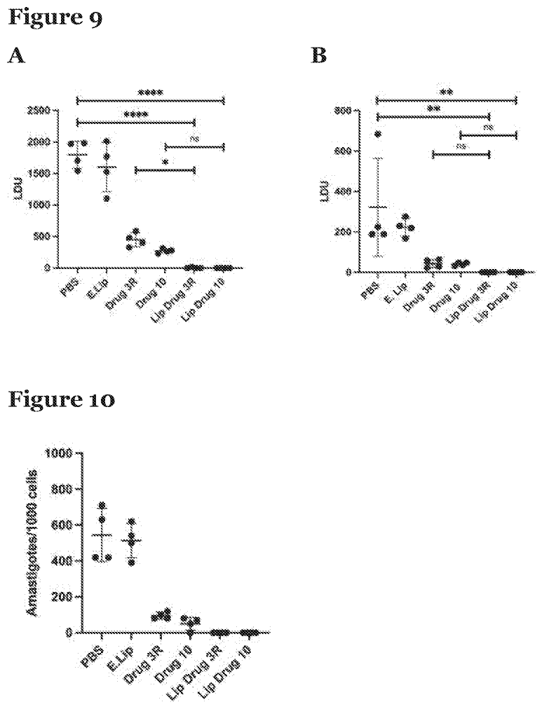

FIG. 9 evaluation of efficacy against visceral leishmaniasis in BALB/c mice in (A) the liver and (B) the spleen through Leishman donovani units (LDU) (n=4);

FIG. 10 evaluation of efficacy against visceral leishmaniasis in BALB/c mice in bone marrow (n=4);

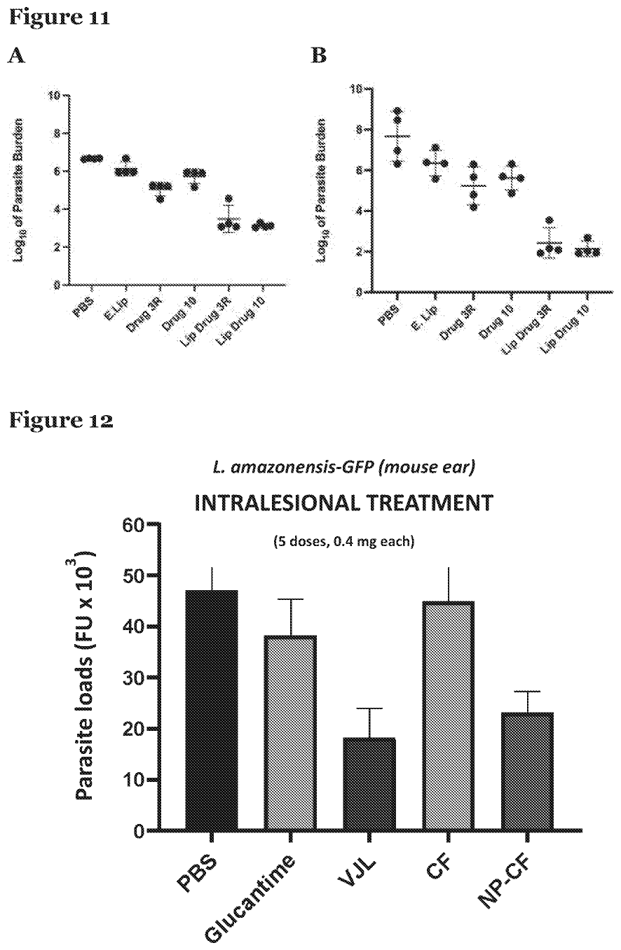

FIG. 11 evaluation of efficacy against visceral leishmaniasis in BALB/c mice in (A) the liver and (B) the spleen through Limiting Dilution Assay (LDA) (n=4);

FIG. 12 evaluation of the parasitic load of intralesional treatment of L. amazonensis-GFP (mouse ear) after treatment with PBS, glucantime, compound 53 (VJL), clemastine fumarate (CF) or clemastine fumarate in a polymeric nanoparticle (NP-CF);

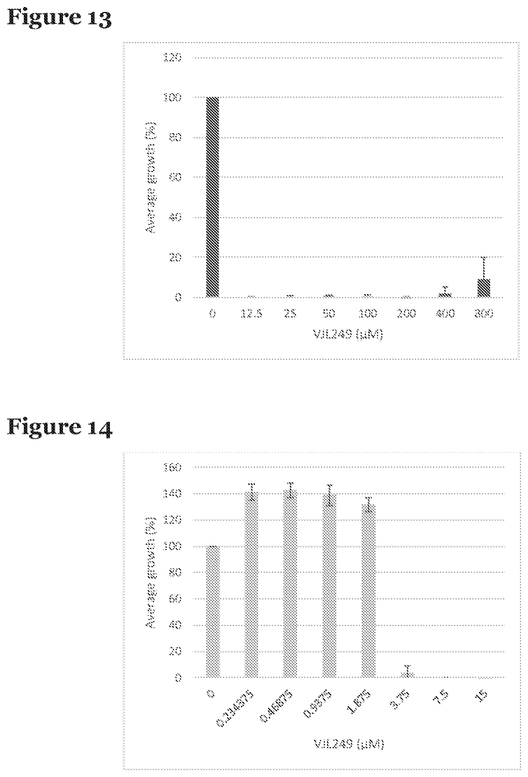

FIG. 13 shows the average growth of S. aureus when treated with a negative control or with concentrations between 12.5 and 800 μM of compound 54 (n=3);

FIG. 14 shows the average growth of S. aureus when treated with a negative control or with concentrations between 0.23 and 15 UM of compound 54 (n=3);

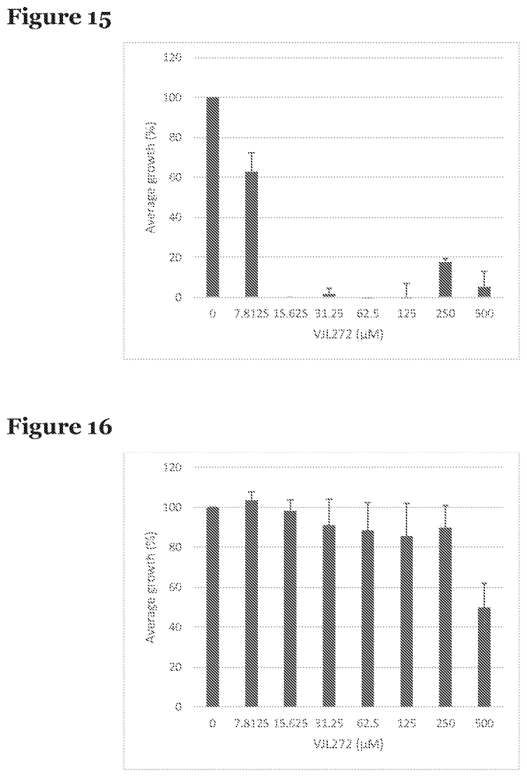

FIG. 15 shows the average growth of S. aureus when treated with a negative control or with concentrations between 7.8 and 500 UM of compound 53 (n=3);

FIG. 16 shows the average growth of wild-type E. coli when treated with a negative control or with concentrations between 7.8 and 500 μM of compound 53 (n=3); and

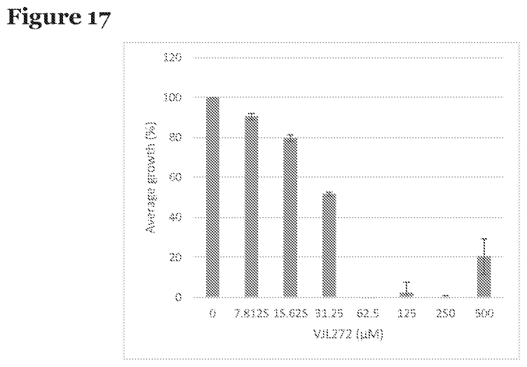

FIG. 17 shows the average growth of ΔrfaC mutant E. coli when treated with a negative control or with concentrations between 7.8 and 500 UM of compound 53 (n=3).

### EXAMPLE 1-ORIGINAL HIT RESYNTHESIS AND VALIDATION

The initial screening pipeline had identified compounds 1 and 2 using L. major inositol phosphoryl ceramide synthase (IPCS) as the target enzyme with subsequent phenotypic validation undertaken using a L. donovani intra-macrophage infection model. In order to connect these observations, initial efforts focused on the resynthesis of the original hit structures and testing in a L. major model. Modified Gewald synthesis provided simple access to the amino thiophene component which could be acylated with 5-nitrothiophene-2-carbonyl chloride (Scheme 1).¹¹⁻¹⁴ Whilst this provided ready access to methyl carboxamide (2) and related analogues 3-5, attempts to generate the parent carboxamide (1) directly were not successful and this had to be generated in a two-step process via hydration of the corresponding nitrile (5).

Using a standard alamarBlue™ assay, activity and selectivity were assessed using L. major promastigotes and HepG2 cells, respectively. All compounds exhibited good (0.47-1.1 μM EC₅₀) activity against the parasite, with the two original carboxamides (1 and 2) and esters (3 and 4) also offering significant selectivity when contrasted with the mammalian cell line (Table 1 entries 1-5). However, as suggested by the tight crystal packing in the solid state (FIG. 1) 1-4 have extremely low aqueous solubility (solubility (PBS)<7.4 μM) which relates to their poor pharmacokinetic profiles.

### Scheme 1. Synthesis of Original Hit Dithiophenes (1 and 2) and Other Dithiophene Analogues (3-5)^{a}

^{a}Reagents and conditions: (a) Et₃N, EtOH, 80° C. (b) Et₃N, THF, 0° C.-RT. (c) 4M HCl_{(aq)}, dioxane, 80° C.

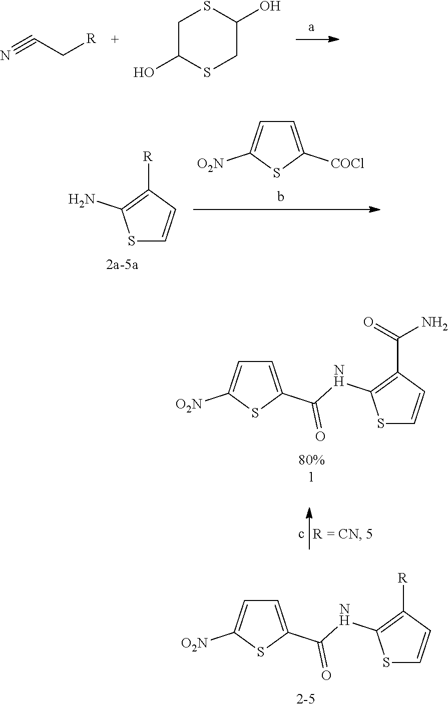

TABLE 1
Antipromastigote, HepG2 toxicity and solubility data for compounds
1 to 5. Averages were calculated from the mean of at least three
biological repeats. Errors represent standard error.

| | | EC₅₀ | EC₅₀ | EC₅₀ | CC₅₀ | Solubility | Solubility |
|---|---|---|---|---|---|---|---|
| | | (L. major)/ | (L. amaz)/ | (L. don)/ | (HepG2)/ | (PBS)/ | (FESSIF)/ |
| ID | R | μM | μM | μM | μM | μM | μM |
| 1 | CONH₂ | 0.52 ± | 0.042 ± | 0.004 ± | 24 ± | <7.4 | 111 ± |
| | | 0.05 | 0.004 | 0.0002 | 1 | | 2 |
| 2 | CONHMe | 0.99 ± | 0.29 ± | 0.033 ± | 96 ± | <7.4 | 35.1 ± |
| | | 0.09 | 0.03 | 0.002 | 4 | | 0.9 |
| 3 | COOMe | 0.59 ± | 0.91 ± | 0.300 ± | >100 | <7.4 | 48 ± |
| | | 0.05 | 0.09 | 0.006 | | | 2 |
| 4 | COOEt | 0.93 ± | 1.85 ± | — | >100 | <7.4 | 29 ± |
| | | 0.09 | 0.05 | | | | 2 |
| 5 | CN | 1.1 ± | 2.1 ± | 1.12 ± | 8 ± | 228 ± | — |
| | | 0.1 | 0.2 | 0.03 | 0.7 | 2 | |

### EXAMPLE 2-5N2C IS ESSENTIAL FOR ANTILEISHMANIAL ACTIVITY

Despite the precedence for nitro drugs to be effective antiparasitic agents, the presence of the nitro group was considered a risk factor and initial attempts explored alternatives for this unit, looking at compounds with general structure 2.

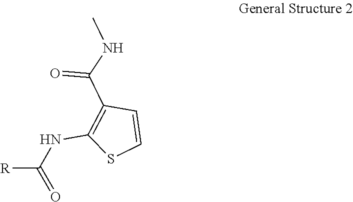

However, removal or replacement of the nitro group leads to a complete loss of antileishmanial activity (6-16, Table 2). Whilst a 5-nitrofuran carboxamide (17, Table 2) retained antileishmanial activity, albeit at the cost of significant HepG2 cytotoxicity, replacement of the nitrothiophene with a 3- or 4-nitro-phenyl group (18-19, Table 2) led to a complete loss of activity. Interestingly, addition of a 2-CF₃ group to the 4-nitrophenyl analogue (20, Table 2) gave a >3 fold improvement in antileishmanial activity compared to 19. The inability to successfully replace or remove the 5N2C group suggested that it was important for antileishmanial activity and should be retained in all future analogues.

TABLE 2
Antipromastigote and HepG2 toxicity data for compounds 6 to
20. Averages were calculated from the mean of at least three
biological repeats. Errors represent standard error.

| | | EC₅₀ | EC₅₀ | EC₅₀ | CC₅₀ |
|---|---|---|---|---|---|
| | | (L. major)/ | (L. amaz)/ | (L. don)/ | (HepG2)/ |
| ID | R | μM | μM | μM | μM |
| 6 | Thiophene-2- | >100 | >100 | — | — |
| 7 | 5-bromothiophene-2- | >100 | >100 | — | — |
| 8 | 5-aminothiophene-2- | >100 | — | — | — |
| 9 | 2-chlorothiazole-5- | 33 ± 10 | — | — | — |
| 10 | Ph | 75 ± 5 | 55 ± 5 | — | — |
| 11 | Thiazole-2- | 78 ± 2 | >100 | — | — |
| 12 | Thiazole-5- | 88 ± 6 | 53 ± 3 | — | — |
| 13 | Thiazole-4- | >100 | 93 ± 7 | — | — |
| 14 | Pyridine-4- | 82 ± 1 | 45 ± 5 | — | — |
| 15 | Pyridine-3- | 82 ± 7 | 59 ± 2 | — | — |
| 16 | (2,1,3)- | >100 | >100 | — | — |
| | benzoxadiazole-5- | | | | |
| 17 | 5-nitrofuran-2- | 0.37 ± | 0.33 ± | 0.2 ± | 2 ± 0.2 |
| | | 0.009 | 0.03 | 0.001 | |
| 18 | 3-NO₂—Ph— | >100 | >100 | — | — |
| 19 | 4-NO₂—Ph— | >100 | >100 | — | — |
| 20 | 4-NO₂-2-CF₃—Ph— | 16.6 ± 0.2 | 38.7 ± | — | — |
| | | | 0.9 | | |

### EXAMPLE 3-2-AMINOTHIOPHENE PAINS MOTIF CAN BE REPLACED WITH ANILINE DERIVATIVES

As a known PAINS moiety the 2-aminothiophene group was considered a potential risk.¹⁵ Initial attempts to address this whilst concomitantly enhancing solubility through increasing the sp³ character suggested the introduction of a similarly sized cyclopentane ring. These building blocks could be accessed by enamine formation from ethyl 2-oxocyclopentanecarboxylate followed by reduction to the amine and amide coupling to give the diastereomeric esters (21 and 22). Acid (23 and 24) and amide (25) derivatives were accessed by subsequent hydrolysis and amide coupling respectively. Whilst saturated derivates (21-25, Table 3) led to significant loss of antileishmanial activity the cyclopentene derivative 26 retained activity, albeit again at the cost of enhanced HepG2 toxicity.

The higher antileishmanial activity observed with compound 26 suggested that the delocalisation of the central amide into the pendant electron withdrawing group is important for antileishmanial activity and attention turned to similarly substituted aniline derivatives which could be simply accessed by coupling of the relevant amine with 5-nitrothiophene-2-carbonyl chloride. Pleasingly, the carboxamides 27 and 28 retained activity compared to the original hits, albeit at the expense of HepG2 toxicity (Table 3).

Anilino analogues 27 and 28 not only removed the 2-amino thiophene PAINS liability, but also were more synthetically accessible and provided further options for exploring chemical space around the right-hand phenyl ring (FIG. 2). Initially using simple commercial anilines a range of alternative substituents were assessed (29-37, Table 3). Overall, no benefit was found by removing or replacing the pendant electron withdrawing group with neutral or donating substituents with most substituents explored leading to modest drop in antileishmanial activity compared to the original hits. However, significantly increasing the bulk of alkyl substituent on the carboxamide led to complete loss of activity (38 and 39, Table 3). In a second approach the inventors opted to retain the C-2 carboxamide and explore additional substituents. Simple substituted anthranilamides were synthesised by triphosgene mediated cyclisation of the anthranilic acid derivative followed by ring opening with methylamine and amide coupling to afford the desired analogues in modest to good yields (Scheme 2).

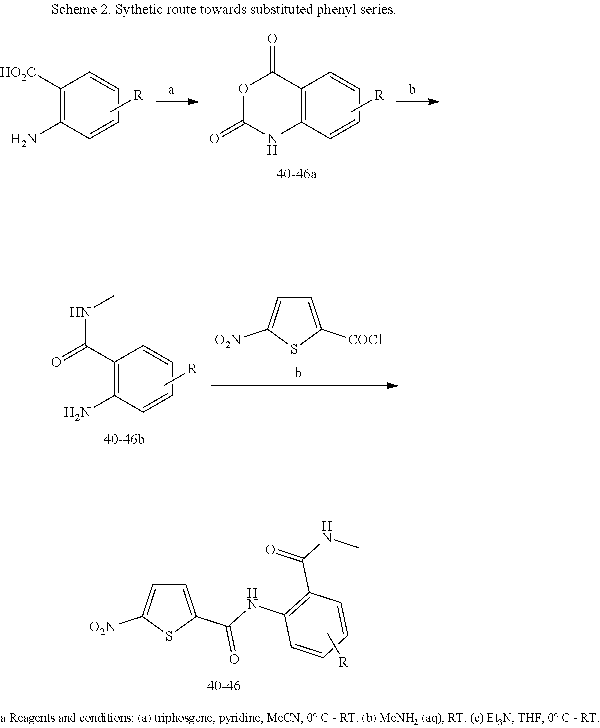

The introduction of a 3- or 4-methyl or methoxy substituents (40-42) resulted in a decrease in antileishmanial activity. In contrast, a 5-substituent (43-46) could be tolerated with optimal activity residing in analogues with the more electron-donating substituents. These (43 and 44) afforded a modest increase in antileishmanial activity compared to the original hits and unsubstituted phenyl analogue 28 (Table 3).

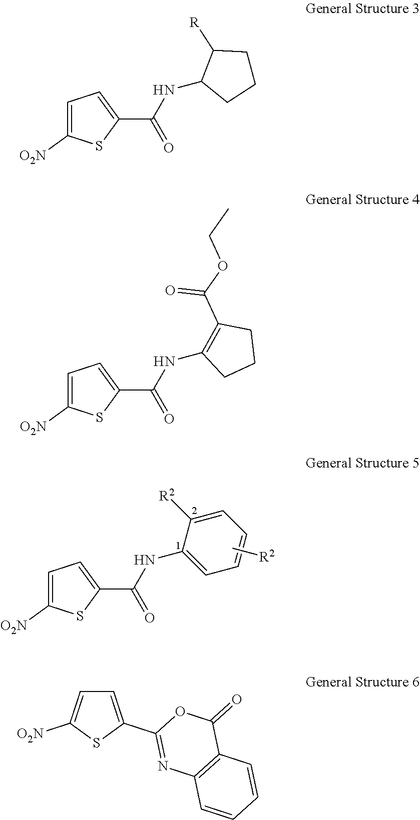

TABLE 3
Antipromastigote, HepG2 toxicity and solubility data for compounds 21 to 46. Averages were calculated
from the mean of at least three biological repeats. Errors represent standard error.

| | | | EC₅₀ | EC₅₀ | EC₅₀ | CC₅₀ | Solubility | Solubility |
|---|---|---|---|---|---|---|---|---|
| General | | | (L. major)/ | (L. amaz)/ | (L. don)/ | (HepG2)/ | (PBS)/ | (FESSIF)/ |
| structure | ID | R | μM | μM | μM | μM | μM | μM |
| 3 | 21 | CO₂Et (syn) | 22 ± | 9.7 ± | — | — | — | — |
| | | | 2 | 0.4 | | | | |
| | 22 | CO₂Et (anti) | 10 ± | 13 ± | — | — | — | — |
| | | | 1 | 1 | | | | |
| | 23 | CO₂H (syn) | >100 | 52 ± | — | — | — | — |
| | | | | 5 | | | | |
| | 24 | CO₂H (anti) | >100 | >100 | — | — | — | — |
| | 25 | CONHMe (anti) | 20 ± | 8.0 ± | 3.40 ± | — | — | — |
| | | | 2 | 0.9 | 0.06 | | | |
| 4 | 26 | — | 0.46 ± | 0.72 ± | — | 2.9 ± | — | — |
| | | | 0.03 | 0.03 | | 0.4 | | |
| 5 | 27 | R¹ = CONH₂ | 0.47 ± | 0.079 ± | — | 4.7 ± | <2.5 | 90 ± |
| | | R² = H | 0.04 | 0.001 | | 0.5 | | 2 |
| | 28 | R¹ = CONHMe | 0.65 ± | 0.052 ± | 0.0120 ± | 5.0 ± | 8.5 ± | 143 ± |
| | | R² = H | 0.05 | 0.002 | 0.0005 | 0.6 | 0.7 | 0.5 |
| | 29 | R¹ = COOH | 46 ± | — | — | — | — | — |
| | | R² = H | 4 | | | | | |
| | 30 | R¹ = OMe | 6.3 ± | — | — | — | — | — |
| | | R² = H | 0.2 | | | | | |
| | 31 | R¹ = CF₃ | 8.3 ± | 4.24 ± | — | — | — | — |
| | | R² = H | 0.6 | 0.1 | | | | |
| | 32 | R¹ = COCH₃ | 16 ± | — | — | — | — | — |
| | | R² = H | 2 | | | | | |
| | 33 | R¹ = H | 4.06 ± | 3.6 ± | 0.93 ± | — | 15 ± | — |
| | | R² = H | 0.06 | 0.4 | 0.03 | | 1 | |
| | 34 | R¹ = H | 3.5 ± | 0.26 ± | — | — | — | — |
| | | R² = 3-CONHMe | 0.4 | 0.03 | | | | |
| | 35 | R¹ = H | 3.9 ± | — | — | — | — | — |
| | | R² = 3-OMe | 0.4 | | | | | |
| | 36 | R¹ = H | 3.45 ± | — | — | — | — | — |
| | | R² = 4-OMe | 0.08 | | | | | |
| | 37 | R¹ = H | 3.5 ± | 2.22 ± | — | — | — | — |
| | | R² = 3-CF₃ | 0.4 | 0.2 | | | | |
| | 38 | R¹ = CONHⁱPr | 4.0 ± | — | — | — | — | — |
| | | R² = H | 0.5 | | | | | |
| | 39 | R¹ = | >100 | — | — | — | — | — |
| | | CONH(CH₂)₁₂CH₃ | | | | | | |
| | | R² = H | | | | | | |
| | 40 | R¹ = CONHMe | 8.5 ± | — | — | — | — | — |
| | | R² = 3-Me | 0.8 | | | | | |
| | 41 | R¹ = CONHMe | 16 ± | — | — | — | — | — |
| | | R² = 4-OMe | 2 | | | | | |
| | 42 | R¹ = CONHMe | 2.7 ± | — | — | — | — | — |
| | | R² = 4-Me | 0.3 | | | | | |
| | 43 | R¹ = CONHMe | 0.33 ± | 0.021 ± | 0.0060 ± | >100 | <7.4 | 38 ± |
| | | R² = 5-OMe | 0.02 | 0.002 | 0.0002 | | | 4 |
| | 44 | R¹ = CONHMe | 0.34 ± | 0.027 ± | 0.0050 ± | 31 ± | <7.4 | 65 ± |
| | | R² = 5-Me | 0.03 | 0.002 | 0.0002 | 4 | | 9 |
| | 45 | R¹ = CONHMe | 1.84 ± | 0.24 ± | — | 13 ± | 6.5 ± | 250 ± |
| | | R² = 5-CF₃ | 0.1 | 0.01 | | 3 | 0.2 | 10 |
| | 46 | R¹ = CONHMe | 0.57 ± | 0.048 ± | 0.12 ± | 7.2 ± | <2.5 | 210 ± |
| | | R² = 5-Br | 0.06 | 0.004 | 0.01 | 0.5 | | 10 |
| | 55 | R¹ = CONHMe | 1.35 ± | 2.5 ± | 1.3 ± | >100 | <2.5 | 260 ± |
| | | R² = 5-^{t}Bu | 0.07 | 0.3 | 0.1 | | | 20 |
| | 56 | R¹ = CONMe₂ | 17 ± | 6.3 ± | — | — | — | — |
| | | R² = H | 2 | 0.9 | | | | |
| 6 | 57 | — | 38 ± | — | — | — | — | — |
| | | | 4 | | | | | |

### EXAMPLE 4-5N2C DERIVATIVES SHOW BROAD SPECTRUM ANTILEISHMANIAL ACTIVITY, LOW MAMMALIAN CELL TOXICITY BUT POOR AQUEOUS SOLUBILITY

At this stage the inventors had identified 13 potent antileishmanial compounds with EC₅₀<2 μM vs L. major (Tables 1 to 3). However, leishmaniasis is a collection of diseases cause by approximately 20 different species of Leishmania parasite that vary according to geographical location and disease manifestation and it is desirable to have pan species activity. Accordingly, the inventors then progressively tested this set of compounds against L. amazonensis promastigotes as a second example of a cutaneous disease-causing species and L. donovani promastigotes as a representative agent of visceral leishmaniasis (FIG. 3). Excitingly, all compounds showed broad spectrum antileishmanial activity. Counter screening against HepG2 cells, allowed the selection of 6 compounds which showed both good levels of activity against all three species and a S.I. (HepG2/L. major)>30 (FIG. 3B). However, a major limitation in these assays was the poor aqueous solubility (solubility (PBS)<7.4 μM) of all the compounds, with the test solutions requiring supplementing with 15% FBS to provide reproduceable values. Improved levels of solubility could be obtained using a biorelevant buffer mimicking the human fed state intestinal fluid (FESSIF) (Tables 1 and 3). However, the poor inherent aqueous solubility of these compounds challenged assays in the more clinically relevant intramacrophage amastigote lifestage and would limit bioavailability if the compounds were to reach animal models.

### EXAMPLE 5-CENTRAL AMIDE IS IMPORTANT FOR ANTILEISHMANIAL ACTIVITY

As attempts to remove the pendant amide group led to a decrease in antileishmanial activity, the inventors then turned to explore the central amide region. Attributing the lack of solubility to the highly conjugated nature of the scaffold, suggested disrupting the sp2 framework through introduction of a spacer unit as seen with 47.

Whilst 47 exhibited higher solubility this was accompanied by a significant decrease in antileishmanial activity (Table 4). In a second approach, it was noted that X-ray analysis of the initial lead compound 2 (FIG. 1) revealed that the close packing nature of the solid state was enabled by a highly planar structure maintained by an intramolecular hydrogen bond between the pendant carboxamide and central amide. Consequently, to disrupt this hydrogen bonding, a methylated central amide analogue (48) was generated by triphosgene cyclisation of 2-aminothiophene-3-carboxylate followed by methylation, methylamine mediated ring opening and amide coupling to give the desired product 48.

This methylation allowed for an alternative folded geometry (FIG. 4), leading to significantly enhanced solubility (Table 4).

However, again this occurred with a 10-fold loss of potency, suggesting that conformational rigidity is required (Table 4). Disappointingly, attempts to retain conformational control by introducing a tetrahydroquinoline ring (49) had a similar outcome. Collectively this suggested that the H-bond enforced planar framework was essential for activity. Finally, the inventors explored the corresponding imine (50) in which an alternative H-bond would be possible between the imine nitrogen and the carboxamide NH. Whist this provided improved solubility and retention of antileishmanial activity and moderate selectivity this appeared to have lower stability with difficulties in purification challenging further progression of this series (Table 4).

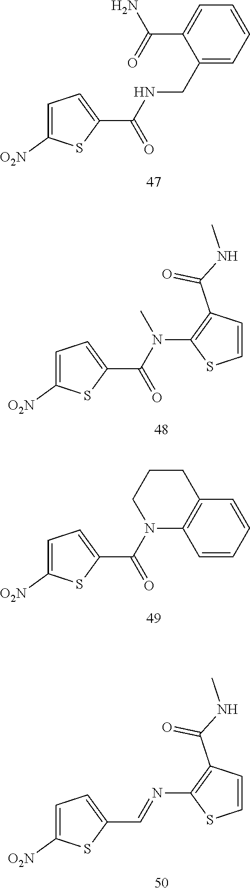 [SMILES: NC(=O)c1ccccc1CNC(=O)c1ccc([N+](=O)[O-])s1 ; CNC(=O)c1ccsc1N(C)C(=O)c1ccc([N+](=O)[O-])s1 ; O=C(c1ccc([N+](=O)[O-])s1)N1CCCc2ccccc21 ; CNC(=O)c1ccsc1/N=C/c1ccc([N+](=O)[O-])s1]

TABLE 4
Antileishmanial activity, selectivity and solubility of
analogues where the central amide has been altered. Averages
were calculated from the mean of at least three biological
repeats. Errors represent standard error.

| Compound | 47 | 48 | 49 | 50 |
|---|---|---|---|---|
| EC₅₀ (L. major)/μM | 21 ± 3 | 9.6 ± 0.7 | 11.1 ± 0.3 | 0.86 ± 0.07 |
| S.I. (HepG2/L. major) | — | 4.0 ± 0.9 | — | 21.9 ± 0.8 |
| Aqueous solubility | 179 ± 8 | 190 ± 20 | 151 ± 3 | 101 ± 6 |
| (PBS/μM) | | | | |

### EXAMPLE 6-5-SUBSTITUENT ON THE PHENYL SERIES CAN BE MANIPULATED TO IMPROVE AQUEOUS SOLUBILITY

In an alternative approach to disrupt the solid-state framework and induce solubility, efforts turned towards the 5-position of the phenyl ring. Analysis of the structure of the original hits suggested that the close packing of the aromatic rings may be contributing to the low solubility. However, attempts to disrupt this thorough the introduction of a 5-tert butyl group (55) had little effect on the crystal packing (FIG. 4B) or solubility. Consequently, attention turned to explore incorporating solubility enhancing groups based on modified PEG units, synthesised by boron tribromide mediated demethylation of 5-methoxy anthranilamide 43b followed by alkylation with the corresponding alkyl bromide (Scheme 3).

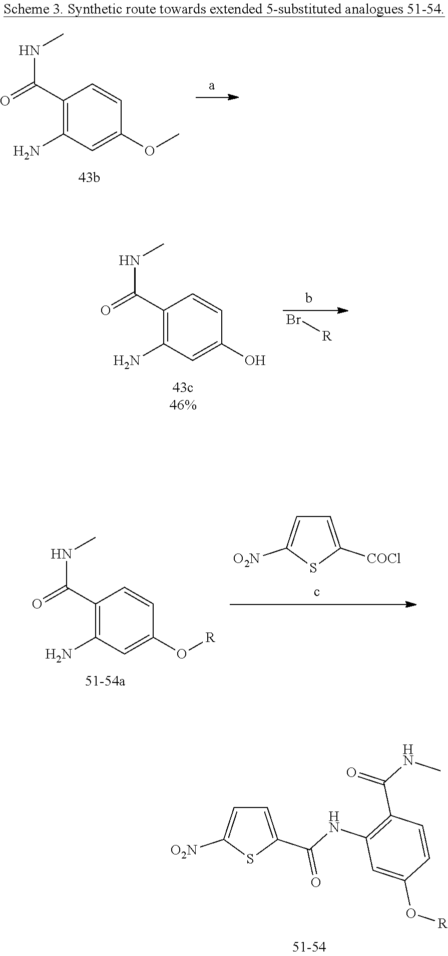

TABLE 5
Yield of compounds 51 to 54

| | Intermediate | Product (B) |
|---|---|---|
| R | A (yield %) | ID (yield %) |
| (C₂H₄)O)₂CH₃ | 51a (27%) | 51 (75%) |
| (C₂H₄)morpholin-4-yl | 52a (18%) | 52 (31%) |
| (C₃H₆)morpholin-4-yl | 53a (57%) | 53 (48%) |
| (C₂H₄)oxan-4-yl | 54a (85%) | 54 (57%) |

Whilst the simple PEG analogue 51 retained antileishmanial activity and high selectivity, it failed to improve aqueous solubility (Table 6). However, introducing a basic substituent in the form of a morpholine ring (52 and 53) provided high antipromastigote activity, high selectivity and improved aqueous solubility (Table 6). Consistent with this observation, X-ray analysis revealed that whereas the PEG linker in 51 could be efficiently accommodated within the lattice (FIG. 5A), the morpholine moiety enforced a much-expanded structure (FIG. 5B). That the basic nitrogen was essential for the enhanced solubility was verified by the fact that the isosteric pyran 54 exhibited significantly reduced aqueous solubility (Table 6). The aqueous solubility of the new lead morpholine compounds could be further improved in biorelevant buffers (FESSIF, FASSIF and FASSGF), suggesting that the lead compounds may be suitable for oral administration (Table 6).

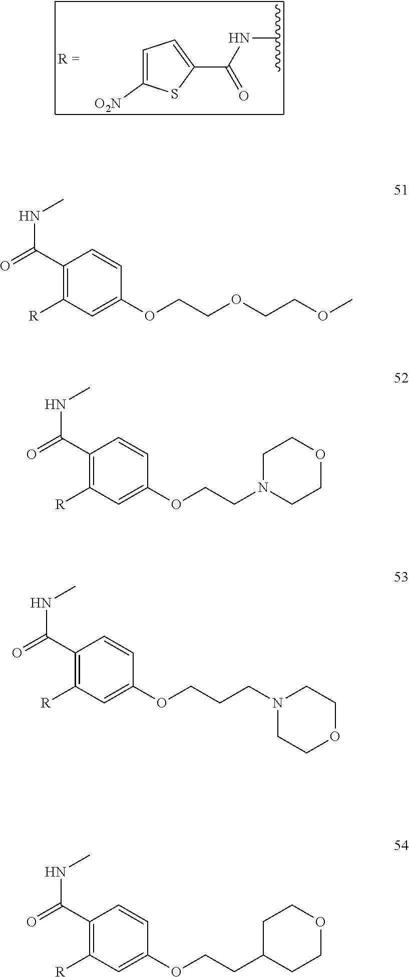

TABLE 6
Antileishmanial activity, HepG2 cytotoxicity and aqueous solubility of
extended 5-substituted 5N2Cs. Averages were calculated from the mean
of at least three biological repeats. Errors represent standard error.

| | EC₅₀ | EC₅₀ | EC₅₀ | CC₅₀ | Solubility | Solubility | Solubility | Solubility |
|---|---|---|---|---|---|---|---|---|
| | (L. major)/ | (L. amaz)/ | (L. don)/ | (HepG2)/ | (PBS)/ | (FESSIF)/ | (FASSIF)/ | (FASSGF)/ |
| ID | μM | μM | μM | μM | μM | μM | μM | μM |
| 51 | 0.32 ± | 0.22 ± | 0.19 ± | >100 | 2.27 ± | 7.26 ± | <7.4 | 11 ± |
| | 0.02 | 0.03 | 0.02 | | 0.05 | 0.04 | | 1 |
| 52 | 0.44 ± | 0.27 ± | 0.134 ± | 24 ± | 19.3 ± | 1870 ± | 148 ± | >8000 |
| | 0.006 | 0.01 | 1 | 1 | 0.6 | 60 | 3 | |
| 53 | 0.30 ± | 0.08 ± | 0.044 ± | 13 ± | 29 ± | 4700 ± | 359 ± | >8000 |
| | 0.02 | 0.01 | 5 | 2 | 3 | 400 | 6 | |
| 54 | 0.33 ± | 0.46 ± | 0.34 ± | >100 | 3.7 ± | 47 ± | — | — |
| | 0.06 | 0.04 | 0.02 | | 0.9 | 4 | | |

### EXAMPLE 7-LEAD MORPHOLINE 5N2CS (52 AND 53) SHOW HIGH INTRAMACROPHAGE ANTILEISHMANIAL ACTIVITY AND SHOW PAN-ACTIVITY AGAINST ANTITRYPANOSOMATIDS

Following the identification of the two lead morpholine 5N2Cs 52 and 53, it became of interest to verify whether they retain activity in the more clinically relevant intra-macrophage amastigote form, and against related trypanosomatids T. brucei and T. cruzi, and the results are provided below.

TABLE 7
Antitrypanosomatid activity of extended 5-substituted
5N2Cs and intramacrophage amastigote activity. Averages
were calculated from the mean of at least three biological
repeats. Errors represent standard error.

| | EC₅₀ | EC₅₀ |
|---|---|---|
| ID | (T. cruzi)/μM | (T. brucei)/μM |
| 52 | 13 ± 1 | 0.78 ± 0.4 |
| 53 | 3.8 ± 0.5 | 0.29 ± 0.1 |

As can be seen in the table, the compounds were also active against the more clinically relevant intra-macrophage amastigote form, and against related trypanosomatids T. brucei and T. cruzi.

The ability of compound 53 to kill intracellular amastigotes for cells infected with L. amazonensis was assessed and the cytotoxicity of the compound was investigated. The results are shown in FIGS. 6 to 8 and Table 8 below.

TABLE 8
Activity, cytotoxicity and selectivity
indexes for compound 53 and pentostam

| | | | |
|---|---|---|---|
| | EC₅₀ | CC₅₀ | |
| Drug | (amastigote) | (macrophage) | SI |

| | | | | | |
|---|---|---|---|---|---|
| 53 | 0.6 ± 0.2 | μg/mL | 16.0 ± 1.0 | μg/mL | 24.6 |
| Pentostam | 16.5 ± 1.2 | μg/mL | ~1284 | μg/mL | ~77.8 |

The selectivity index (SI) was calculated by dividing the half maximal cytotoxic concentration (CC₅₀) by the half maximal effective concentration (EC₅₀). CC₅₀ was extrapolated for pentostam.

As can be seen from the above data, compound 53 is active at much lower amounts than pentostam (an existing treatment for leishmaniasis). Additionally, compound 53 is shown to have activity at concentrations which are significantly lower than the cytotoxic dose. This is reflected in the relatively high selectivity index for the compound.

### EXAMPLE 8-IN VIVO EFFICACY OF COMPOUNDS AGAINST EXPERIMENTAL VISCERAL LEISHMANIASIS

Mice were infected with 2×10⁷ promastigotes and kept for 2 months. After 2 months mice were treated with a single dose of phosphate-buffered saline (PBS), an empty liposome formulation (E.Lip), 10 mg/kg of body weight of Drug 3R, 10 mg/kg of body weight of compound 53 (identified in the Figures as drug 10), a liposome composition comprising 10 mg/kg of body weight of Drug 3R (Lip Drug 3R) or a liposome composition comprising 10 mg/kg of body weight of compound 53 (Lip Drug 10). Drug 3R is a reference compound which does not fall within the scope of the invention. The mice were then kept for one month and then sacrificed. Infection in liver and spleen was determined through LDU and LDA (FIGS. 9 and 11), infection in bone marrow was determined by counting amastigotes/1000 cells (FIG. 10).

It will be noted that compound 53 alone has reasonable antileishmanial effect but when delivered (iv) in a liposomal formulation then very effective clearance at this dosing is observed.

### EXAMPLE 9-IN VIVO EFFICACY OF COMPOUND 53 AGAINST EXPERIMENTAL L. AMAZONENSIS-GFP (MOUSE EAR)

Mice were infected with L. amazonensis and were then treated intralesionally (IL) with five doses of 10 μL PBS only (control) or 10 μL of PBS containing 0.4 mg of glucantime, compound 53 (VJL), clemastine fumarate (CF) or clemastine fumarate in a polymeric nanoparticle (NP-CF). The mice were treated once a week for five weeks. The results are shown in FIGS. 12 to 14, and again shown that compound 53 is an effective treatment, outperforming glucatime, clemastine fumarate and clemastine fumarate in a polymeric nanoparticle.

### EXAMPLE 10-IN VITRO EFFICACY OF COMPOUNDS AGAINST S. AUREUS (A GRAM POSITIVE BACTERIA)

The ability of compounds 53 and 54 to inhibit the growth of S. aureus was investigated and the results are shown in FIGS. 13 and 14 (compound 54) and FIG. 15 (compound 53). It is noted that both compounds were effective at inhibiting the bacteria at low concentrations.

Example 11-In vitro efficacy of compound 53 against E. coli (a gram negative bacteria) The ability of compound 53 to inhibit the growth of both wild-type and ΔrfaC mutant E. coli was investigated and the results are shown in FIG. 16 (wild-type) and FIG. 17 (ΔrfaC mutant). There is little effect against the wild-type, but some reasonable activity against the ΔrfaC mutant. It is noted that the ΔrfaC mutant has a defect in to the membrane than allows permeation of the drug. Accordingly, the lack of activity in WT E. coli is due to a transport phenomena rather than an activity defect. This indicates that compounds of the invention could also be used to treat gram negative bacteria.

### Experimental Section

### General Procedures

All reagents were purchased from commercial suppliers. All solvents were dry unless otherwise stated and were either dried in house or purchased from commercial suppliers. Reactions were monitored by thin layer chromatography (TLC) using Merck silica gel aluminium sheets (F254) and visualised under UV light (254 nm) using a UVP Mineralight® lamp or by staining with permanganate, ninhydrin, Mary's reagent or phosphomolybdic acid. Melting points were recorded on Thermo Scientific Electrothermal IA9100 Digital Melting Point apparatus. Nuclear magnetic resonance (NMR) spectra were recorded on the following instruments: Varian VNMRS-600 instrument with operating frequencies of 600.130 MHz for 1H and 150.903 MHz for 13C NMR, Vari-an VNMRS-700 with operating frequencies of 700.130 MHz for 1H and 176.048 MHz for 13C NMR, Avance III-HD-400, Bruker spectrometer-400 and Bruker Neo-400. Spectra were referenced relative to CDCl₃ (8×7.26 ppm, &c 77.16 ppm), DMSO-do (δ_{H} 2.50 ppm, &c 39.52 ppm) or CD OD (δ_{H} 4.87 ppm, & 49.00 ppm). Chemical shifts are reported in parts per million (ppm), coupling constants (J) in hertz (Hz) and multiplicity as singlet(s), doublet (d), triplet (t), quartet (q), multiplet (m) or a combination thereof. All ¹H NMR and 3C NMR spectral assignments were made with the aid of ¹H-¹H COSY, ¹H-¹H NOESY, ¹H-¹³C HSQC and ¹H-¹³C HMBC NMR experiments. Ar-refers to an aromatic carbon or proton that could not be as-signed. Infra-red (IR) spectra were recorded on a PerkinElmer Paragon™ 1000 FT-IR spectrometer or a Perki-nElmer Frontier™ FT-IR with Golden Gate Diamond ATR apparatus. 1R assignments are reported in wavenumbers (cm⁻¹) and may be assigned as broad (br) or weak (w). High resolution mass spectrometry (HRMS) and LC-MS were recorded on either a Waters TQD mass spectrometer with Acquity UPLC (ESI-LC water (0.1% formic acid): MeCN, flow rate 0.6 mL min⁻¹ with a UPLC BEH C18 1.7 μm (2.1 mm×50 mm) column), Waters QtoF Premier mass spectrometer with Acquity UPLC (ESI-LC water (0.1% formic acid): MeCN, flow rate 0.2 mL min⁻¹ with a UPLC BEH C18 1.7 μm (2.1 mm×100 mm) column) or a Waters SQD mass spectrometer with Acquity UPLC (ESI-LC water (0.1% formic acid): MeCN, flow rate 0.6 mL min⁻¹ with a UPLC BEH C¹⁸ 1.7 μm (2.1 mm×50 mm) column). GCMS was carried out on a Shimadzu QP2010-Ultra with a temperature gradient 50° C.-300° C. and a hold time of 5 mins, using a Rxi-17Sil MS (0.15 μm×10 m×0.15 mm) column. ASAP was carried out by dipping a melting point tube into sample solution. Samples are run isothermally at 350° C. on a Waters LCT Premier XE with Acquity UPLC or at 450° C. on a Waters Xevo QTOF mass spectrometer.

### General Procedure A

Oxalyl chloride (1.2 eq.) was added dropwise to a solution of carboxylic acid in dry dichloromethane (DCM, 1-2 mL per 1 mmol) under argon at 0° C., followed by 1 drop of dimethylformamide (DMF). The reaction was stirred at room temperature (RT) for 2 hrs before volatiles were removed in vacuo to give the desired acid chloride.

### General Procedure C

5-nitrothiophene-2-carbonyl chloride (23) (unless otherwise specified, 1.2 eq.) was dissolved in dry tetrahydrofuran (THF) or DCM (as specified) and added dropwise to a solution of substituted amine (1.0 eq.) and triethylamine (2.5 eq.) in dry THF or DCM at 0° C. under argon. The reaction mixture was stirred at RT for 2 hrs (unless otherwise specified). Product was filtered and triturated with water, unless otherwise stated, to give the title amide.

### General Procedure G

5-nitrothiophene-2-carbonyl chloride (23) (1.2 eq.), unless otherwise stated, was dissolved in dry THF or DCM (stated) and added dropwise to a solution of substituted amine (1.0 eq.) and triethylamine (2.5 eq.) in dry THF or DCM at 0° C. under argon and stirred for 20 hr at RT. Volatiles were removed and the residue extracted into EtOAc, washed with saturated sodium bicarbonate (general procedure Gi) or 1M HCl (general procedure Gii), water and brine and purified as specified to afford the title product.

### General Structures

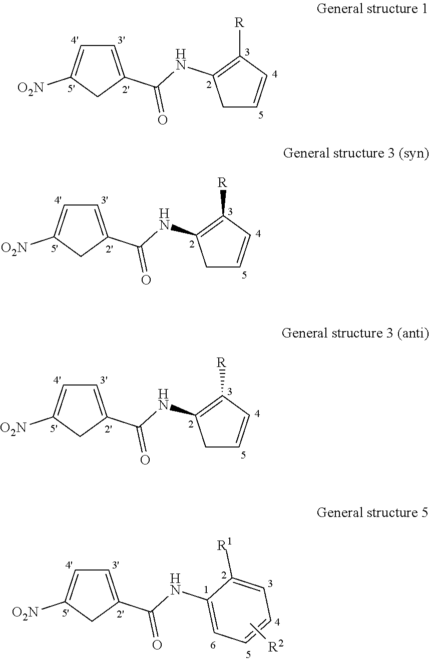

### N-(3-cyanothiophen-2-yl)-5′-nitrothiophene-2′-carboxamide (5)

5-nitrothiophene-2-carbonyl chloride (0.61 g, 3.2 mmol, 1.0 eq.) was dissolved in dry THF (3 mL) and added dropwise to a solution of 2-amino-3-cyanothiophene (0.50 g, 3.9 mmol, 1.2 eq.), DIPEA (0.67 mL, 3.8 mmol, 1.2 eq.) and DMAP (0.004 g, 0.03 mmol, 0.01 eq.) in dry THF (5 mL) under argon at 0° C. The reaction mixture was stirred at RT for 22 hrs. Volatiles were removed in vacuo and the residue was purified by flash column chromatography (EtOAc:hexane) to give N-(3-cyanothiophen-2-yl)-5′-nitrothiophene-2′-carboxamide (5) as an orange/yellow solid (0.45 g, 50%) melting point 251-253° C. (dec.). νₘₐₓ (ATR) 3266 (N—H), 2230 (C≡N), 1564 (C═O), 1501, 1285 cm⁻¹. δ_{H} (599 MHz, DMSO-d₆) 12.28 (1H, s, NH), 8.21 (1H, d, J 4.5, 4′—H), 8.14 (1H, d, J 4.5, 3′—H), 7.37 (1H, d, J 5.8, 5-H), 7.28 (1H, d, J 5.8, 4-H). δ_{C} (151 MHz, DMSO-d₆) 158.3 (C═O), 154.2 (C-2′), 147.8 (C-2), 142.6 (C-5′), 130.4 (C-3′), 129.9 (C-4′), 125.9 (C-4), 121.3 (C-5), 114.5 (C≡N), 96.7 (C-3). m/z (LCMS ES⁻) 278.1 [M−H]⁻. HRMS (ES⁻) found [M−H]⁻ 277.9682, C₁₀H₄N₃O₃S, requires M 277.9694. CHN found: C 50.17; H 5.23; N 9.05. C₁₀H₉N₃O₃S2 requires C 49.99; H 5.16; N 8.97.

### N-(3-carbamoylthiophen-2-yl)-5′-nitrothiophene-2′-carboxamide (1)

N-(3-cyanothiophen-2-yl)-5′-nitrothiophene-2′-carboxamide (36) (0.207 g, 0.72 mmol, 1 eq.) was dissolved in 1,4 dioxane (16 mL) and stirred with 4M HCl (aq) (5.6 mL, 22.3 mmol, 31 eq.) at 80° C. under argon for 72 hrs. The reaction mixture cooled on ice for 1 hr and the resulting precipitate was filtered and dried in vacuo to give N-(3-carbamoylthiophen-2-yl)-5′-nitrothiophene-2′-carboxamide (1) as an orange-yellow solid (0.17 g, 80%) melting point 293-296° C. (dec.). νₘₐₓ (ATR) 3477 (N—H), 3376 (N—H), 1646 (C═O), 1593, 1508, 1338 cm⁻¹. δ_{H} (599 MHz, DMSO-d₆) 13.64 (1H, s, 2-NH), 8.20 (1H, d, J 4.4, 4′—H), 8.14 (1H, s, NH₂), 7.78 (1H, s, NH₂), 7.68 (1H, d, J 4.4, 3′—H), 7.51 (1H, d, J 5.8, 4-H), 7.13 (1H, d, J 5.8, 5-H). δ_{C} (151 MHz, DMSO-d₆) 167.2 (3-C═O), 155.9 (2′—C═O), 153.9 (C-2′), 144.9 (C-2), 142.9 (C-5′), 130.3 (C-4′), 128.2 (C-3′), 123.3 (C-4), 117.6 (C-5), 116.6 (C-3). m/z (LCMS ES⁺) 298.1 [M+H]⁺. HRMS (ES⁺) found [M+H]⁺ 297.9961, C₁₀H₇N₃O₄S₂ requires M 297.9956. CHN found: C 40.40; H 2.42; N 13.78. C₁₀H₇N₃O₄S₂ requires C 40.40; H 2.37; N 14.13.

### N-methyl-2-(5′-nitrothiophene-2′-amido)thiophene-3-carboxamide (2)

Following general procedure C, using THF (10 mL) as a reaction solvent, where the substituted amine was 2-amino-N-methylthiophene-3-carboxamide (0.23 g, 1.5 mmol, 1.0 eq.) afforded N-methyl-2-(5′-nitrothiophene-2′-amido)thiophene-3-carboxamide (2) as a yellow solid (0.24 g, 52%) melting point 248° C. (dec.). νₘₐₓ (ATR) 3442 (N—H), 3087 (N—H), 1657 (C═O), 1627 (C═O), 1541, 1335 cm⁻¹. δ_{H} (599 MHz, DMSO-d₆) 13.61 (1H, s, 2-NH), 8.62 (1H, s, CH₃-NH), 8.20 (1H, d, J 4.4, 4′—H), 7.72 (1H, d, J 4.4, 3′—H), 7.49 (1H, d, J 5.8, 4-H), 7.15 (1H, d, J 5.8, 5-H), 2.83 (3H, d, J 4.5, CH₃). δ_{C} (151 MHz, DMSO-d₆) 165.5 (3-C═O), 155.9 (2′—C═O), 153.9 (C-2′), 144.2 (C-2), 142.8 (C-5′), 130.3 (C-4′), 128.3 (C-3′), 122.5 (C-4), 117.9 (C-5), 116.6 (C-3), 25.8 (CH₃). m/z (LCMS ES⁺) 312.1 ([M+H]⁺). HRMS (ES⁺) found [M+H]⁺ 312.0120, C₁₁H₁₀N₃O₄S₂ requires M 312.0113. CHN found: C 42.61; H 2.99; N 13.29. C₁₁H₉N₃O₄S2 requires C 42.44; H 2.91; N 13.50.

### Methyl 2-(5′-nitrothiophene-2′-amido)thiophene-3-carboxylate (3)

Following general procedure C, using DCM (10 mL) as a reaction solvent, where the substituted amine was methyl 2-aminothiophene-3-carboxylate (0.50 g, 3.5 mmol, 1.0 eq.) afforded methyl 2-(5′-nitrothiophene-2′-amido)thiophene-3-carboxylate (3) as a yellow solid (0.45 g, 41%) melting point 228-229° C. νₘₐₓ (ATR) 3220 (N—H), 3100 (N—H), 1675 (C═O), 1649 (C═O), 1566, 1335 cm⁻¹. δ_{H} (599 MHz, DMSO-d₆) 11.74 (1H, s, 2-NH), 8.22 (1H, d, J 4.4, 4′—H), 7.85 (1H, d, J 4.4, 3′—H), 7.28 (1H, d, J 5.8, 4-H), 7.21 (1H, d, J 5.8, 5-H), 3.90 (3H, s, CH₃). δ_{C} (151 MHz, DMSO-d₆) 165.2 (3-C═O), 157.2 (2′-C═O), 154.8 (C-2′), 146.9 (C-2), 142.7 (C-5′), 130.7 (C-4′), 129.6 (C-3′), 124.5 (C-4), 119.2 (C-5), 115.1 (C-3), 52.1 (CH₃). m/z (LCMS ES⁺) 313.1 [M+H]⁺. HRMS (ES⁺) found [M+H]⁺ 312.9968, C₁₁H₉N₂O₅S₂ requires M 312.9953. CHN found: C 42.29; H 2.59; N 8.95. C₁₁H₇N₂O₅S₂ requires C 42.30; H 2.58; N 8.97.

### Ethyl 2-(5′-nitrothiophene-2′-amido)thiophene-3-carboxylate (4)

Following general procedure Gi, using DCM (5 mL) as a reaction solvent, where the substituted amine was 2-aminothiophene-3-ethanoate (0.39 g, 2.3 mmol, 0.8) afforded, following work up with sodium hydroxide and purification using flash column chromatography (DCM:hexane), ethyl 2-(5′-nitrothiophene-2′-amido)thiophene-3-carboxylate (4) as a yellow solid (0.21 g, 28%) melting point 212-213° C. νₘₐₓ (ATR) 3119 (N—H), 1722 (C═O), 1646 (C═O), 1508, 1329 cm⁻¹. δ_{H} (700 MHz, CDCl₃) 12.14 (1H, s, N—H), 7.93 (1H, d, J 4.3, 4′—H), 7.65 (1H, d, J 4.3, 3′—H), 7.29 (1H, d. J 5.8, 4-H), 6.86 (1H, d, J 5.8, 5-H), 4.40 (2H, q, J 7.1, CH₂), 1.42 (3H, t, J 7.1, CH₃). δ_{C} (176 MHz, CDCl₃) 166.2 (OC═O), 156.8 (NHC═O), 155.6 (C-2′), 147.9 (C-2), 142.9 (C-5′), 128.4 (C-4′), 127.3 (C-3′), 124.3 (C-4), 117.4 (C-5), 114.5 (C-3), 61.4 (CH₂), 14.5 (CH₃). m/z (LCMS ES⁺) 327.2 [M+H]⁺. HRMS (ES⁺) found [M+H]⁺ 327.0110, C₁₂H₁₁N₂O₅S₂ requires M 327.0109. CHN found: C 44.04; H 3.12; N 8.59. C₁₂H₉N₂O₅S₂ requires C 44.16; H 3.09; N 8.58.

### N-methyl-2-(thiophene-2′-amido)thiophene-3-carboxamide (6)

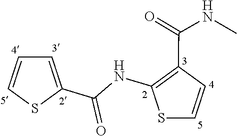 [SMILES: CNC(=O)c1ccsc1NC(=O)c1cccs1]

Following general procedure C, using THF (8 mL) as a reaction solvent for 7 hrs, where the acid chloride was thiophene-2-carbonyl chloride (0.23 g, 1.60 mmol, 1.25 eq.) and the substituted amine was 2-amino-N-methylthiophene-3-carboxamide (0.20 g, 1.28 mmol, 1 eq.) afforded, following removal of volatiles from the filtrate followed by flash column chromatography (hexane:EtOAc) and recrystallisation (hexane: DCM), N-methyl-2-(thiophene-2′-amido)thiophene-3-carboxamide (6) as a white crystalline solid (0.14 g, 40%) melting point 191-192° C. νₘₐₓ (ATR) 3445 (N—H), 3405 (N—H), 1651 (C═O), 1624 (C═O) cm⁻¹. δ_{H} (700 MHz, CDCl₃) 12.90 (1H, s, 2-NH), 7.81 (1H, dd, J 3.8, J 1.1, 5′—H), 7.60 (1H, dd, J 5.0, J 1.1, 3′—H), 7.15 (1H, dd, J _{3′, 4′} 5.0, J _{4′, 5′} 3.8, 4′—H), 6.95 (1H, dd, J 5.8, J 0.4, 4-H), 6.82 (1H, dd, J 5.8, J 0.8, 5-H), 5.99 (1H, s, CH₃—NH), 3.04 (3H, d, J 4.9, CH₃). δ_{C} (176 MHz, CDCl₃) 166.4 (3-C═O), 158.8 (2′-C═O), 147.2 (C-2), 137.7 (C-2′), 132.0 (C-3′), 129.6 (C-5′), 128.2 (C-4′), 120.4 (C-4), 117.1 (C-5), 114.6 (C-3), 26.5 (CH₃). m/z (LCMS ES⁺) 267.5 [M+H]⁺. HRMS (ES⁺) found [M+H]⁺ 267.0265, C₁₁H₁₁N₂O₂S₂ requires M 267.0262. CHN found: C 49.58; H 3.70; N 10.43. C₁₁H₁₀N₂O₂S₂ requires C 49.61; H 3.78; N 10.52.

### 5′-bromo-N-[3-(methylcarbamoyl)thiophen-2-yl]thiophene-2′-carboxamide (7)

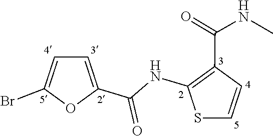 [SMILES: CNC(=O)c1ccsc1NC(=O)c1ccc(Br)o1]

Following general procedure C, using THF (10 mL) as a reaction solvent for 15 hr, where the acid chloride was 5-bromothiophene-2-carbonyl chloride (2.26 g, 10 mmol, 1.2 eq.) and the substituted amine was 2-amino-N-methylthiophene-3-carboxamide (1.31 g, 8.3 mmol, 1.0 eq.) afforded, following removing volatiles from the filtrate and purification by flash column chromatography (50:50 EtOAc:Hexane) followed by recrystallisation (EtOAc:Hexane), 5′-bromo-N-[3-(methylcarbamoyl)thiophen-2-yl]thiophene-2′-carboxamide (7) as an off-white solid (0.66 g, 23%) melting point 171-172° C. νₘₐₓ (ATR) 3382 (N—H), 1639 (C═O), 1629 (C═O), 1552, 1321. δ_{H} (599 MHz, CDCl₃) 12.92 (1H, s, 2-NH), 7.53 (1H, d, J 4.0, 4-H), 7.10 (1H, d, J 4.0, 5-H), 6.98 (1H, d, J 5.8, 4′—H), 6.81 (1H, dd, J 5.8 J 0.8, 1H), 6.15 (1H, d, J 4.7 CH₃—NH), 3.01 (3H, d, J 4.7, CH₃). δ_{C} (151 MHz, CDCl₃) 166.4 (2′-C═O), 157.6 (3-C═O), 146.8 (C-2′), 139.0 (C-2), 131.3 (C-5), 129.6 (C-4), 120.6 (C-4′), 120.1 (C-3), 117.2 (C-3′), 114.9 (C-5′), 26.5 (CH₃). m/z (LCMS ES⁺) 345.0 [M(⁷⁹Br)+H]⁺& 347.0 [M(⁸¹Br)+H]⁺. HRMS (ES⁺) found [M+H]⁺ 344.9380, C₁₁H₁₀N₂O₂S₂Br requires M 344.9367. CHN found: C 38.13; H 2.60; N 8.10. C₁₁H₉N₂O₂S₂Br requires C 38.27; H 2.63; N 8.11.

### N-[3-(methylcarbamoyl)thiophen-2-yl]-5′-nitrofuran-2′-carboxamide (17)

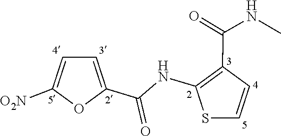 [SMILES: CNC(=O)c1ccsc1NC(=O)c1ccc([N+](=O)[O-])o1]

Following general procedure C, using THF (6 mL) as a reaction solvent, where the acid chloride was 5-nitrofuran-2-carbonyl chloride (0.44 g, 2.5 mmol, 1.2 eq.) and the substituted amine was 2-amino-N-methylthiophene-3-carboxamide (0.33 g, 2.1 mmol, 1.0 eq.) afforded, following recrystallisation (DCM:hexane), N-[3-(methylcarbamoyl)thiophen-2-yl]-5′-nitrofuran-2′-carboxamide (17) as a yellow solid (0.062 g, 10%) melting point 233-234° C. νₘₐₓ (ATR) 3466 (N—H), 3300 (N—H), 1671 (C═O), 1660 (C═O) 1556, 1355 cm⁻¹. δ_{H}(599 MHz, DMSO-d₆) 13.43 (1H, s, 2-NH), 8.55 (1H, d, J 4.6, CH₃—NH), 7.85 (1H, d, J 3.9, 4′—H), 7.58 (1H, d, J 3.9, 3′—H), 7.49 (1H, d, J 5.8, 4-H), 7.16 (1H, d, J 5.8, 5-H), 2.84 (3H, d, J 4.6, CH₃). δ_{C} (151 MHz, DMSO-d₆) 165.2 (3-C═O), 152.5 (2′—C═O), 151.7 (C-5′), 146.3 (C-2′), 143.7 (C-2), 122.5 (C-4), 117.9 (C-3′ & C-5), 116.9 (C-3), 113.8 (C-4′), 25.8 (CH₃). m/z (LCMS ES⁺) 296.1 [M+H]⁺. HRMS (ES⁺) found [M+H]⁺ 296.0370, C₁₁H₁₀N₃O₅S requires M 296.0341. CHN found: C 44.32; H 2.99; N 14.01. C₁₁H₉N₃O₅S requires C 44.74; H 3.07; N 14.23.

### N-methyl-2-[4′-nitro-2′-(trifluoromethyl)benzamido]thiophene-3-carboxamide

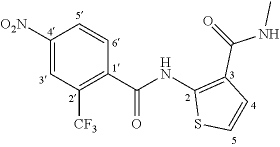 [SMILES: CNC(=O)c1ccsc1NC(=O)c1ccc([N+](=O)[O-])cc1C(F)(F)F]

Following general procedure Gi, using THF (6 mL) as a reaction solvent, where the acid chloride was 4-nitro-2-(trifluoromethyl)benzoyl chloride (0.49 g, 1.92 mmol, 1.2 eq.) and the substituted amine was 2-amino-N-methylthiophene-3-carboxamide (0.49 g, 1.92 mmol, 1.2 eq.) afforded, following work up using chloroform and purification by flash column chromatography (EtOAc/hexane), N-methyl-2-[4′-nitro-2′-(trifluoromethyl)benzamido]thiophene-3-carboxamide (20) as a yellow solid (0.39 g, 65%), melting point 170-172° C. νₘₐₓ (ATR) 3442 (N—H), 1682 (C═O), 1629 (C═O), 1553, 1351 cm⁻¹. δ_{H} (700 MHz, CDCl₃) 12.66 (1H, s, 2-NH), 8.64 (1H, d, J 2.2, 3′—H), 8.51 (1H, dd, J 8.3 J 2.2, 5′—H), 7.91 (1H, d, J 8.3, 6′—H), 7.00 (1H, d, J 5.9, 4-H), 6.93 (1H, dd, J 5.9, 5-H), 6.04 (1H, s, NHCH₃), 2.97 (3H, d, J 4.9, CH₃). δ_{C} (176 MHz, CDCl₃) 166.0 (3-C═O), 162.0 (1′—C═O). 148.7 (C-4′), 145.8 (C-2), 139.8 (C-1′), 130.5 (C-6′), 130.2 (q, J 34.2, C-2′), 127.2 (C-5′), 122.6 (q, J 5.1, C-3′), 122.3 (q, J 268.2, CF₃), 120.5 (C-4), 118.1 (C-5), 116.1 (C-3), 26.4 (CH₃). OF (376 MHz, CDCl₃) −59.4 (CF₃). m/z (LCMS ES⁺) 374.2 [M+H]⁺. HRMS (ES⁺) found [M+H]⁺ 374.0422, C₁₄H₁₁N₃O₄F₃S requires 374.0422. CHN found: C 45.17; H 2.75; N 11.16. C₁₄H₁₂N₃O₄F₃S requires C 45.04; H 2.70; N 11.25.

### N-[3-(methylcarbamoyl)thiophen-2-yl]-1′,3′-thiazole-2′-carboxamide (11)

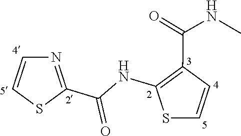 [SMILES: CNC(=O)c1ccsc1NC(=O)c1nccs1]

Following general procedure A, where the acid was 1,3-thiazole-2-carboxylic acid (0.200 g, 1.5 mmol, 1.0 eq.), afforded 1,3-thiazole-2-carbonyl chloride as a pale-yellow solid, which was used without purification or characterisation.

Following general procedure C, using THF (6 mL) as a reaction solvent, where the acid chloride was 1,3-thiazole-2-carbonyl chloride (0.20 g, 1.35 mmol, 1.2 eq.) and the substituted amine was 2-amino-N-methylthiophene-3-carboxamide (0.18 g, 1.13 mmol, 1.0 eq.) afforded, following removal of volatiles from the filtrate and purification of the residue by flash column chromatography (hexane:EtOAc) followed by recrystallisation (hexane:EtOAc), N-[3-(methylcarbamoyl)thiophen-2-yl]-1′,3′-thiazole-2′-carboxamide (11) as a pale yellow solid (0.073 g, 21%) melting point 212-213° C. νₘₐₓ (ATR) 3330 (N—H), 3228 (N—H), 3111 (N—H) 1666 (C═O), 1619 (C═O) cm⁻¹. δ_{H} (700 MHz, DMSO-d₆) 13.52 (1H, s, 2-NH), 8.48 (1H, d, J 4.7, CH₃—NH), 8.21 (1H, d, J 3.0, 5′—H), 8.17 (1H, d, J 3.0, 4′—H), 7.48 (1H, d, J 5.8, 4-H), 7.14 (1H, dd, J 5.8 J 0.8, 5-H), 2.82 (3H, d, J 4.7, CH₃). δ_{C} (176 MHz, DMSO-d₆) 165.0 (3-C═O), 161.2 (C-2′), 155.9 (2′-C═O), 144.6 (C-4′), 143.8 (C-2), 127.5 (C-5′), 122.6 (C-4), 117.5 (C-5), 117.0 (C-3), 25.7 (CH₃). m/z (LCMS ES⁺) 268.2 [M+H]⁺. HRMS (ES⁺) found [M+H]⁺ 268.0226, C₁₀H₁₀N₃O₂S₂ requires M 268.0214. CHN found: C 45.38; H 3.56; N 15.39. C₁₀H₉N₃O₂S₂ requires C 44.93; H 3.39; N 15.72.

### N-[3-(methylcarbamoyl)thiophen-2-yl]-1′,3′-thiazole-5′-carboxamide (12)

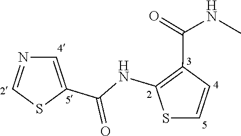 [SMILES: CNC(=O)c1ccsc1NC(=O)c1cncs1]

Following general procedure A, where the acid was 1,3-thiazole-5-carboxylic acid (0.200 g, 1.5 mmol, 1.0 eq.), afforded 1,3-thiazole-5-carbonyl chloride as an off white solid, which was used without purification or characterisation.

Followed general procedure C, using THF (6 mL) as a reaction solvent, where the acid chloride was 1,3-thiazole-5-carbonyl chloride (0.20 g, 1.35 mmol, 1.2 eq.) and the substituted amine was 2-amino-N-methylthiophene-3-carboxamide (0.18 g, 1.13 mmol, 1.0 eq.), afforded N-[3-(methylcarbamoyl)thiophen-2-yl]-1′,3′-thiazole-5′-carboxamide (12) as an off-white solid (0.091 g, 30%) melting point 277-278° C. νₘₐₓ (ATR) 3308 (N—H), 3077 (N—H), 1660 (C═O), 1623 (C═O) cm⁻¹. δ_{H} (700 MHz, DMSO-d₆) 13.32 (1H, s, 2-NH), 9.39 (1H, s, 2′-H), 8.55 (1H, d, J 4.7, CH₃—NH), 8.47 (1H, s, 4′-H), 7.47 (1H, d, J 5.8, 4-H), 7.10 (1H, d, J 5.8, 5-H), 2.83 (1H, d, J 4.7, CH₃). δ_{C} (176 MHz, DMSO-d₆) 165.5 (3-C═O), 159.5 (C-2′), 156.3 (5′-C═O), 145.0 (C-4′), 144.6 (C-2), 133.0 (C-5′), 122.4 (C-4), 117.2 (C-5), 116.0 (C-3), 25.8 (CH₃). m/z (LCMS ES⁺) 268.1 [M+H]⁺. HRMS (ES⁺) found [M+H]⁺ 268.0222, C₁₀H₁₀N₃O₂S₂ requires M 268.0214. CHN found: C 44.98; H 3.51; N 15.26. C₁₀H₉N₉O₂S₂ requires C 44.93; H 3.39; N 15.72.

### N-[3-(methylcarbamoyl)thiophen-2-yl]-1′,3′-thiazole-4′-carboxamide (13)

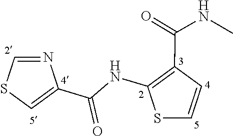 [SMILES: CNC(=O)c1ccsc1NC(=O)c1cscn1]

Following general procedure A, where the acid was 1,3-thiazole-4-carboxylic acid (0.200 g, 1.5 mmol, 1.0 eq.), afforded 1,3-thiazole-4-carbonyl chloride (100) as an off white solid, which was used without purification or characterisation.

Following general procedure C, using THF (6 mL) as a reaction solvent, where the acid chloride was 1,3-thiazole-4-carbonyl chloride (0.20 g, 1.35 mmol, 1.2 eq.) and the substituted amine was 2-amino-N-methylthiophene-3-carboxamide (0.18 g, 1.13 mmol, 1.0 eq.), afforded N-[3-(methylcarbamoyl)thiophen-2-yl]-1′,3′-thiazole-4′-carboxamide (13) as an off-white solid (0.13 g, 38%) melting point 272-273° C. νₘₐₓ (ATR) 3375 (N—H), 3111 (N—H), 1657 (C═O), 1639 (C═O) cm⁻¹. δ_{H} (599 MHz, DMSO-d₆) 13.40 (1H, s, 2-NH), 9.32 (1H, d, J 2.1, 2′—H), 8.60 (1H, d, J 2.1, 5′—H), 8.40 (1H, d, J 4.7, CH₃—NH), 7.45 (1H, d, J 5.9, 4-H), 7.07 (1H, d, J 5.9, 5-H), 2.80 (3H, d, J 4.7, CH₃). δ_{C} (151 MHz, DMSO-d₆) 165.0 (3-C═O), 157.3 (4′-C═O), 156.0 (C-2′), 148.6 (C-4′), 144.3 (C-2), 126.7 (C-5′), 122.5 (C-4), 117.0 (C-5), 116.3 (C-3), 25.7 (CH₃). m/z (LCMS ES⁺) 268.1 [M+H]⁺. HRMS (ES⁺) found [M+H]⁺ 268.0223, C₁₀H₁₀N₃O₂S₂ requires M 268.0214. CHN found: C 44.81; H 3.46; N 15.44. C₁₀H₉N₃O₂S₂ requires C 44.93; H 3.39; N 15.72.

### N-[3-(methylcarbamoyl)thiophen-2-yl]-2,1,3-benzoxadiazole-5′-carboxamide (16)

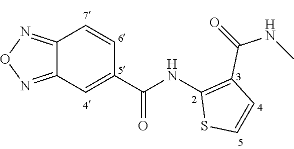 [SMILES: CNC(=O)c1ccsc1NC(=O)c1ccc2nonc2c1]

Following general procedure C, using THF (2 mL) as a reaction solvent, where the acid chloride was 2,1,3-benzoxadiazole-5-carbonyl chloride (0.22 g, 1.2 mmol, 1.2 eq.) and the substituted amine was 2-amino-N-methylthiophene-3-carboxamide (0.16 g, 1.13 mmol, 1.0 eq.), afforded N-[3-(methylcarbamoyl)thiophen-2-yl]-2,1,3-benzoxadiazole-5′-carboxamide (16) as a yellow solid (0.062 g, 20%) melting point 248-249° C. νₘₐₓ (ATR) 3410 (N—H), 3081 (N—H), 1661 (C═O), 1621 (C═O), 1555, 1311 cm⁻¹. δ_{H} (599 MHz, DMSO-d₆) 13.58 (1H, s, 2-NH), 8.56-8.59 (2H, m, 4′-H & NH—CH₃), 8.28 (1H, dd, J 9.4 J 1.1, 7′—H), 7.95 (1H, dd, J 9.4 J 1.5, 6′—H), 7.48 (1H, d, J 5.9, 4-H), 7.13 (1H, d, J 5.9, 5-H), 2.83 (3H, d, J 4.5, CH₃). δ_{C} (151 MHz, DMSO-d₆) 165.5 (3-C═O), 160.7 (5′—C═O), 149.2 (C-8′), 148.8 (C-3′), 144.8 (C-2), 136.2 (C-5′), 130.0 (C-6′), 122.5 (C-4), 117.6 (C-7′), 117.5 (C-5), 116.7 (C-4′), 116.5 (C-3), 25.8 (CH₃). m/z (LCMS ES⁺) 303.2 [M+H]⁺. HRMS (ES⁺) found [M+H]⁺ 303.0556, C₁₃H₁₁N₄O₃S requires M 303.0552. CHN found: C 51.21; H 3.40; N 18.05. C₁₃H₁₀N₄O₃S requires C 51.65; H 3.33; N 18.53.

### 2-benzamido-N-methylthiophene-3-carboxamide (10)

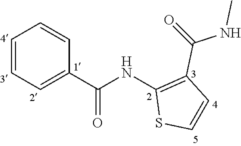 [SMILES: CNC(=O)c1ccsc1NC(=O)c1ccccc1]

Following general procedure Gi, using THF (3 mL) as a reaction solvent, where the acid chloride was benzoyl chloride (0.10 mL 0.84 mmol, 1.2 eq.) and the substituted amine was 2-amino-N-methylthiophene-3-carboxamide (0.11 g, 0.70 mmol, 1.0 eq.) afforded, following recrystallisation from DCM:hexane, 2-benzamido-N-methylthiophene-3-carboxamide (10) as an off white solid (0.0378, 20%) melting point 183-184° C. νₘₐₓ (ATR) 3374 (N—H), 3330 (N—H), 1661 (C═O), 1635 (C═O), 1555, 1405 cm⁻¹. δ_{H} (599 MHz, CDCl₃) 13.03 (1H, s, 2-NH), 8.05-8.08 (2H, m, 2′-H), 7.56-7.59 (1H, m, 4′-H), 7.50-7.54 (2H, m, 3′-H), 6.99 (1H, d, J 5.7, 4-H), 6.83 (1H, dd, J 5.7, J 0.8, 5-H), 6.07 (1H, s, CH₃—NH), 3.03 (3H, d, J 4.9, CH₃). δ_{C} (151 MHz, CDCl₃) 166.3 (3-C═O), 163.7 (1′—C═O), 147.3 (C-2), 132.5 (C-4′), 132.3 (C-1′), 128.9 (C-3′), 127.6 (C-2′), 120.4 (C-4), 116.9 (C-5), 114.7 (C-3), 26.3 (CH₃). m/z (LCMS ES⁺) 261.2 [M+H]⁺. HRMS (ES⁺) found [M+H]⁺ 261.0682, C₁₃H₁₃N₂O₂S requires M 261.0698. CHN found: C 59.23; H 4.70; N 10.69. C₁₃H₁₂N₂O₂S requires C 59.98; H 4.65; N 10.76.

### N-[3-(methylcarbamoyl)thiophen-2-yl]pyridine-4′-carboxamide (14)

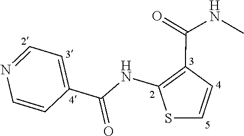 [SMILES: CNC(=O)c1ccsc1NC(=O)c1ccncc1]

Isonicotinoyl chloride (0.11 g, 0.77 mmol, 1.2 eq.) dissolved in dry DMF (3 mL) was added to 2-amino-N-methylthiophene-3-carboxamide (0.100 g, 0.64 mmol, 1.0 eq.) dissolved in dry THF (7 mL) and triethylamine (0.089 mL, 1.9 mmol, 2.5 eq.) at 0° C. under argon and the reaction was stirred at RT for 23 hr. Volatiles were removed in vacuo before extraction into EtOAc (20 mL) and washed with water (3×20 mL) and brine (20 mL) before drying over MgSO₄. Purification was achieved by flash column chromatography (EtOAc:hexane) followed by recrystallisation (CHCl₃/hexane) to give N-[3-(methylcarbamoyl)thiophen-2-yl]pyridine-4′-carboxamide (14) (0.043 g, 26%) as an off white solid melting point 230-231° C. νₘₐₓ (ATR) 3297 (N—H), 3088 (N—H), 1658 (C═O), 1627 (C═O) cm⁻¹. δ_{H} (599 MHz, CDCl₃) 13.28 (1H, s, C2-NH), 8.80-8.88 (2H, m, 2′-H), 7.88 (2H, d, J 5.9, 3′—H), 7.00 (1H, d, J 5.8, 4-H), 6.90 (1H, d, J 5.8, 5-H), 6.13 (1H, s, NH—CH₃), 3.04 (3H, d, J 4.9, CH₃). δ_{C} (151 MHz, CDCl₃) 166.4 (CH₃-C═O), 161.9 (4′-C═O), 151.1 (C-2′), 146.6 (C-2), 139.6 (C-4′), 121.2 (C-3′), 120.6 (C-4), 117.7 (C-5), 115.6 (C-3), 26.5 (CH₃). m/z (LCMS ES⁺) 262.2 [M+H]⁺. HRMS (ES⁺) found [M+H]⁺ 262.0658, C₁₂H₁₂N₃O₂S requires M 262.0650. CHN found: C 54.69; H 4.20; N 15.96. C₁₂H₁₁N₃O₂S requires C 55.16 H 4.24; N 16.08.

### N-[3-(methylcarbamoyl)thiophen-2-yl]pyridine-3′-carboxamide (15)

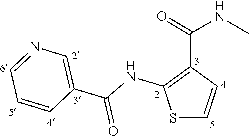 [SMILES: CNC(=O)c1ccsc1NC(=O)c1cccnc1]

Pyridine-3-carbonyl chloride (0.11 g, 0.77 mmol, 1.2 eq.) dissolved in dry DMF (3 mL) was added to 2-amino-N-methylthiophene-3-carboxamide (0.10 g, 0.64 mmol, 1.0 eq.) and triethylamine (0.089 mL, 1.9 mmol, 2.5 eq.) dissolved in dry THF (3 mL) at 0° C. under argon and the reaction was stirred at RT for 19 hrs. Volatiles were removed in vacuo and the residue was extracted into EtOAc (20 mL) and washed with water (4×20 mL) and brine (1×20 mL) before drying over MgSO₄. The solvent was removed in vacuo and the resulting solid was purified by trituration in DCM and water to give N-[3-(methylcarbamoyl)thiophen-2-yl]pyridine-3′-carboxamide (15) as an off-white semi-solid (0.016 g, 10%). νₘₐₓ (ATR) 3315 (N—H), 3088 (N—H), 1656 (C═O), 1630 (C═O). δ_{H} (599 MHz, DMSO-d₆) 13.49 (1H, s, 2-NH), 9.10 (1H, d, J 2.3, 2′—H), 8.84 (1H, dd, J 4.8 J 1.8, 6′—H), 8.56 (1H, d, J 4.7, CH₃—NH), 8.26-8.28 (1H, m, 4′-H), 7.67 (1H, ddd, J 7.9 J 4.8 J 0.9, 5′—H), 7.48 (1H, d, J 5.8, 4-H), 7.11 (1H, d, J 5.8, 5-H), 2.83 (3H, d, J 4.7, CH₃). δ_{C} (176 MHz, DMSO-d₆) 165.6 (3-C═O), 161.1 (3′—C═O), 153.1 (C-6′), 148.1 (C-2′), 145.1 (C-2), 134.8 (C-4′), 128.0 (C-3′), 124.2 (C-5′), 122.4 (C-4), 117.1 (C-5), 116.1 (C-3), 25.8 (CH₃). m/z (LCMS ES⁺) 262.2 [M+H]⁺. HRMS (ES⁺) found [M+H]⁺ 262.0660, C₁₂H₁₂N₃O₂S requires M 262.0650.

### N-methyl 2-(4′-nitrobenzamido)thiophene-3-carboxamide (19)

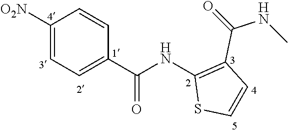 [SMILES: CNC(=O)c1ccsc1NC(=O)c1ccc([N+](=O)[O-])cc1]

Following general procedure C, using THF (10 mL) as a reaction solvent for 72 hrs, where the acid chloride was 4-nitrobenzoyl chloride (0.29 g, 1.54 mmol, 1.2 eq.) and the substituted amine was 2-amino-N-methylthiophene-3-carboxamide (0.20 g, 1.3 mmol, 1.0 eq.) afforded, following removal of volatiles from the filtrate, purification by flash column chromatography (EtOAc: hexane) and recrystallisation (EtOAc:hexane), N-methyl-2-(4′-nitrobenzamido)thiophene-3-carboxamide (19) as a yellow solid (0.013 g, 3%) melting point 276-277° C. νₘₐₓ (ATR) 3405 (N—H), 3117 (N—H), 1662 (C═O), 1619 (C═O), 1560, 1352 cm⁻¹. δ_{H} (700 MHz, DMSO-d₆) 13.59 (1H, s, 2-NH), 8.60 (1H, d, J 4.7, CH₃NH), 8.47 (2H, d, J 8.8, 3′—H), 8.17 (2H, d, J 8.8, 2′—H), 7.50 (1H, d, J 5.8, 4-H), 7.14 (1H, d, J 5.8, 5-H), 2.84 (3H, d, J 4.7, CH₃). δ_{C} (176 MHz, DMSO-d₆) 165.6 (3-C═O), 160.9 (1′—C═O), 149.7 (C-1′), 145.0 (C-2), 137.2 (C-4′), 128.6 (C-2′), 124.4 (C-3′), 122.4 (C-4), 117.2 (C-5), 116.2 (C-3), 25.8 (CH₃). m/z (LCMS ES⁺) 306.1 [M+H]⁺. HRMS (ES⁺) found [M+H]⁺ 306.0552, C₁₃H₁₂N₃O₄S requires M 306.0549.

### N-methyl-2-(3′-nitrobenzamido)thiophene-3-carboxamide (18)

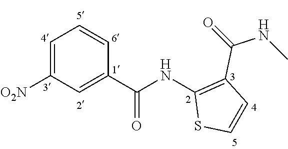 [SMILES: CNC(=O)c1ccsc1NC(=O)c1cccc([N+](=O)[O-])c1]

Following general procedure C, using THF (8 mL) as a reaction solvent for 3 hrs, where the acid chloride was 3-nitrobenzoyl chloride (0.29 g, 1.54 mmol, 1.3 eq.) and the substituted amine was 2-amino-N-methylthiophene-3-carboxamide (0.20 g, 1.3 mmol, 1.0 eq.), afforded N-methyl-2-(3′-nitrobenzamido)thiophene-3-carboxamide (18) as a yellow solid (0.032 g, 9%) melting point 260-261° C. νₘₐₓ (ATR) 3449 (N—H), 3431 (N—H), 1660 (C═O), 1620 (C═O), 1550, 1357 cm⁻¹. δ_{H} (599 MHz, DMSO-d₆) 13.60 (1H, s, 2-NH), 8.65-8.68 (1H, m, 5′-H), 8.58 (1H, s, NH—CH₃), 8.50 (1H, d, J 8.2, 4′—H), 8.31-8.34 (1H, m, 6′-H), 7.92-7.97 (1H, m, 2′-H), 7.49 (1H, d, J 5.8, 4-H), 7.12 (1H, d, J 5.8, 5-H), 2.84 (3H, d, J 4.5, NH—CH₃). δ_{C} (151 MHz, DMSO-d₆) 165.6 (3-C═O), 160.4 (1′—C═O), 148.2 (C-1′), 145.0 (C-2), 133.6 (C-3′), 132.9 (C-6′), 131.1 (C-2′), 127.0 (C-4′), 122.4 (C-4), 121.9 (C-5′), 117.3 (C-5), 116.3 (C-3), 25.8 (CH₃). m/z (LCMS ES⁺) 306.1 [M+H]⁺. HRMS (ES⁺) found [M+H]⁺ 306.0554, C₁₃H₁₂N₃O₄S requires M 306.0549. CHN found: C 51.16; H 3.69; N 13.56. C₁₃H₁₁N₃O₄S requires C 51.14: H 3.63; N 13.76.

### 5′-amino-N-[3-(methylcarbamoyl)thiophen-2′-yl]thiophene-2-carboxamide (8)

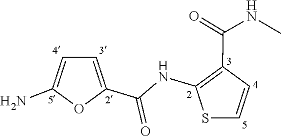 [SMILES: CNC(=O)c1ccsc1NC(=O)c1ccc(N)o1]

Pd/C (0.070 g, 0.6 mmol, 0.5 eq.) was wet with a catalytic amount of EtOAc and degassed before N-methyl-2-(5′-nitrothiophene-2′ amido)thiophene-3-carboxamide (0.40 g, 1.3 mmol, 1.0 eq.) in MeOH (8 mL) was added. The reaction was stirred under a H₂ atmosphere (balloon pressure) for 27 hrs before filtering through Celite and removing volatiles in vacuo. Purification by flash column chromatography (CHCl₃:MeOH) gave 5′-amino-N-[3-(methylcarbamoyl)thiophen-2′-yl]thiophene-2-carboxamide (8) as a brown semi-solid (0.15 g, 42%). νₘₐₓ (ATR) 3039-3463 (br, NH₂), 1624 (C═O), 1607 (C═O) cm⁻¹. δ_{H} (700 MHz, MeOD) 7.42 (1H, d, J 4.1, 3′—H), 7.23 (1H, d, J 5.9, 4-H), 6.82 (1H, d, J 5.9, 5-H), 6.08 (1H, d, J 4.1, 4′—H), 2.91 (3H, s, CH₃). δ_{C} (176 MHz, MeOD) 168.2 (3-C═O), 164.4 (2′-C═O), 160.7 (C-5′), 148.0 (C-2), 133.0 (C-3′), 122.7 (C-4), 118.3 (C-2′), 117.0 (C-5), 115.4 (C-3), 107.0 (C-4′), 26.3 (CH₃). m/z (LCMS ES⁺) 282.2 [M+H]⁺. HRMS (ES⁺) found [M+H]⁺ 282.0377, C₁₁H₁₂N₃O₂S₂ requires M 282.0371.

### N-[3-(methylcarbamoyl)thiophen-2-yl]-2′-chloro-1′,3′-thiazole-5′-carboxamide (9)

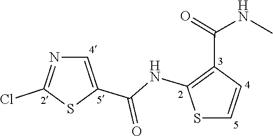 [SMILES: CNC(=O)c1ccsc1NC(=O)c1cnc(Cl)s1]

2-chlorothiazole-5-carbonyl chloride (0.17 g, 0.89 mmol, 1.1 eq.) in dry THF (1 mL) was added to a solution of 2-amino-N-methylthiophene-3-carboxamide (0.13 g, 0.81 mmol, 1.0 eq.) and triethylamine (0.28 mL, 2.1 mmol, 2.5 eq.) in THF at 0° C. under argon and the reaction was stirred at RT for 20 hrs. Volatiles were removed in vacuo and the residue was extracted into EtOAc, washed with 1 M HCl and then extracted into 1M NaOH. The basic layer was washed with EtOAc and reacidified to pH 5 and extracted into EtOAc. The organic layer was washed with brine and dried over MgSO₄. Volatiles were removed in vacuo and the crude product was purified by flash column chromatography (EtOAc/hexane) followed by recrystallisation (CHCl₃/hexane) to give N-[3-(methylcarbamoyl)thiophen-2-yl]-2′-chloro-1′,3′-thiazole-5′-carboxamide (9) as a yellow solid (0.008 g, 3%) melting point 210-211° C. νₘₐₓ (ATR) 3337 (N—H), 1667 (C═O), 1621 (C═O) cm⁻¹. δ_{H} (700 MHz, CDCl₃) 13.07 (1H, s, 2-NH), 8.18 (1H, s, 4′-H) 6.97 (1H, d, J 5.9, 4-H), 6.87 (1H, dd, J 5.9, J 0.8, 5-H), 6.01 (1H, s, CH₃—NH), 3.04 (3H, d, J 4.9, CH₃). 0c (176 MHz, CDCl₃) 166.3 (3-C═O), 156.9 (C-2′), 156.2 (5′—C═O), 146.2 (C-2), 142.8 (C-4′), 135.7 (C-5′), 120.5 (C-4), 117.7 (C-5), 115.3 (C-3), 25.5 (CH₃). m/z (LCMS ES⁺) 302.2 [M+H]⁺. HRMS (ES⁺) found [M+H]⁺ 301.9828, C₁₀H₉N₃O₂S₂Cl requires 301.9825.

### ±Ethyl-2-(5′-nitrothiophene-2′-amido)cyclopentane-1-carboxylate (21 & 22)

DIPEA (3.0 mL, 16.9 mmol, 3.0 eq.) was added to a solution of 5-nitrothiophene-2-carboxylic acid (0.99 g, 5.7 mmol, 1.0 eq.) in DMF (8 mL). The solution was cooled to 0° C. and treated with EDCl (2.17 g, 11.3 mmol, 2.0 eq.), HOBt (1.73 g, 11.3 mmol, 2.0 eq.) and a mixture of anti: syn (8:2) ethyl 2-aminocyclopentane-1-carboxylate (1.09 g, 6.9 mmol, 1.2 eq.). The reaction mixture was stirred at RT for 20 hours before dilution with EtOAc (70 mL) and washing with water (3×50 mL). The EtOAc layer was dried over MgSO₄ and purified by flash column chromatography (hexane:EtOAc) to give anti-ethyl-2-(5′-nitrothiophene-2′-amido)cyclopentane-1-carboxylate (22) as a yellow solid (0.53 g, 30%) melting point 111-112° C. νₘₐₓ (ATR) 3325 (N—H), 1731 (C═O), 1634 (C═O), 1511, 1338 cm⁻¹. δ_{H} (599 MHz, CDCl₃) 7.83 (1H, d, J 4.4, 4′—H), 7.41 (1H, d, J 4.4, 3′—H), 6.69 (1H, s, NH), 4.48-4.53 (1H, m, 2-H), 4.13 (2H, q, J 7.2, CH—CH₃), 2.74-2.78 (1H, m, 1-H), 2.21-2.26 (1H, m, 3-H), 2.04-2.08 (1H, m, 5-H), 1.89-1.95 (1H, m, 5-H), 1.76-1.81 (2H, m, 4-H), 1.61-1.67 (1H, m, 3-H), 1.22 (3H, t, J 7.2, CH₃). δ_{C} (151 MHz, CDCl₃) 174.4 (OC═O), 160.0 (NHC═O), 154.2 (C-2′), 145.2 (C-5′), 128.4 (C-4′), 126.2 (C-3′), 61.0 (CH₃-CH₂), 55.6 (C-2), 50.5 (C-1), 32.8 (C-3), 28.5 (C-5), 23.2 (C-4), 14.3 (CH₃). m/z (LCMS ES⁺) 313.2 [M+H]⁺. HRMS (ES⁺) found [M+H]⁺ 313.0862, C₁₃H₁₇N₂O₅S requires M 313.0858. CHN found: C 50.17; H 5.23; N 9.05. C₁₃H₁₆N₂O₅S requires C 49.99; H 5.16; N 8.97. Flash column chromatography followed by recrystallisation (DCM:hexane) also gave syn-ethyl-2-(5′-nitrothiophene-2′-amido)cyclopentane-1-carboxylate (21) as an off-white solid (0.096 g, 5%) melting point 109-110° C. νₘₐₓ (ATR) 3333 (N—H), 1730 (C═O), 1640 (C═O), 1511, 1341 cm⁻¹. δ_{H} (700 MHz, CDC)₃) 7.84 (1H, d, J 4.3, 4′—H), 7.35-7.36 (1H, m, NH), 7.33 (1H, d, J 4.3, 3′—H), 4.55-4.59 (1H, m, 2-H), 4.12-4.19 (2H, m, CH₂-CH₃), 3.03 (1H, dt, J 8.4, J 6.7, 1-H), 2.12-2.14 (1H, m, 3-H), 2.00-2.09 (2H, m, 4-H), 1.86-1.89 (1H, m, 5-H), 1.79-1.83 (1H, m, 3-H), 1.67-1.73 (1H, m, 5-H), 1.26 (3H, t, J 7.1, CH₃). δ_{C} (176 MHz, CDCl₃) 175.5 (OC═O), 159.6 (NHC═O), 154.3 (C-2′), 145.4 (C-5′), 128.3 (C-4′), 125.8 (C-3′), 61.2 (CH₃-CH₂), 52.7 (C-2), 46.0 (C-1), 32.3 (C-3), 29.1 (C-4), 22.5 (C-5), 14.3 (CH₃). m/z (LCMS ES⁺) 313.1 [M+H]⁺. HRMS (ES⁺) found [M+H]⁺ 313.0863, C₁₃H₁₇N₂O₅S requires M 313.0858. CHN found: C 49.11; H 5.08; N 8.73. C₁₃H₁₆N₂O₅S requires C 49.99; H 5.16; N 8.97.

### ±anti 2-(5′-nitrothiophene-2′-amido)cyclopentane-1-carboxylic acid (24)

±anti ethyl-2-(5′-nitrothiophene-2′-amido)cyclopentane-1-carboxylate (0.11 g, 0.35 mmol, 1.0 eq.) was dissolved in MeOH (3 mL) before 1M LiOH (1.8 mL, 1.76 mmol, 5.0 eq.) was added at 0° C. under argon. The reaction mixture was stirred at 0° C. for 7 hours, before acidification to pH 7. MeOH was removed in vacuo and the remaining solution was acidified further to pH 2, before extraction into EtOAc (3×30 mL). The organic layer was washed with brine (2×30 mL) and dried over MgSO₄. Solvent was removed in vacuo to give ±anti 2-(5′-nitrothiophene-2′-amido)cyclopentane-1-carboxylic acid (24) (0.080 g, 88%) as an off white solid, melting point 229-230° C. νₘₐₓ (ATR) 3314 (O—H), 1702 (C═O), 1628 (C═O), 1508, 1314 cm⁻¹. δ_{H} (700 MHz, CD₃OD) 7.96 (1H, d, J 4.3, 4′—H), 7.68 (1H, d, J 4.3, 3′—H), 4.54-4.57 (1H, m, 2-H), 2.81-2.85 (1H, m, 1-H), 2.14-2.18 (1H, m, 3-H), 2.08-2.13 (1H, m, 5-H), 1.89-1.93 (1H, m, 5-H), 1.76-1.87 (2H, m, 4-H), 1.65-1.71 (1H, m, 3-H). δ_{C} (176 MHz, CD₃OD) 178.2 (0-C═O), 162.0 (N—C═O), 155.4 (C-2′), 146.7 (C-5′), 129.8 (C-4′), 128.1 (C-3′), 56.4 (C-2), 51.0 (C-1), 33.5 (C-3), 30.0 (C-5), 24.4 (C 4). m/z (LCMS ES⁺) 285.2 [M+H]⁺. HRMS (ES⁺) found [M+H]⁺ 285.0560, C₁₁H₁₃N₂O₅S requires M 285.0545. CHN found: C 46.66; H 4.49; N 9.35. C₁₁H₁₂N₂O₅S requires C 46.47; H 4.25; N 9.85.

### ±syn 2-(5′-nitrothiophene-2′-amido)cyclopentane-1-carboxylic acid (23)

±syn ethyl-2-(5′-nitrothiophene-2′-amido)cyclopentane-1-carboxylate (0.055 g, 0.2 mmol, 1.0 eq.) was dissolved in MeOH (2 mL) before 1M LiOH (1.1 mL, 1.1 mmol, 5.0 eq.) was added at 0° C. under argon. The reaction mixture was stirred at 0° C. for 5 hours, before acidification to pH 7. MeOH was removed in vacuo and the remaining solution was acidified further to pH 2, before extraction into EtOAc (3×30 mL). The organic layer was washed with brine (2×30 mL) and dried over MgSO₄. Volatiles were removed in vacuo to give ±syn 2-(5′-nitrothiophene-2′-amido)cyclopentane-1-carboxylic acid (23) (0.05 g, 88%) as an off white solid, melting point 201-202° C. νₘₐₓ (ATR) 3316 (O—H), 3097 (N—H), 1698 (C═O), 1620 (C═O), 1558, 1344 cm⁻¹. OH (599 MHz, CD₃OD) 7.95 (1H, d, J 4.4, 4′—H), 7.68 (1H, d, J 4.4, 3′—H), 4.60 (1H, q, J 7.6, 2-H), 3.11 (1H, q, J 7.6, 1-H), 2.05-2.11 (2H, m, aliph-H), 1.84-2.00 (3H, m, aliph-H), 1.62-1.70 (1H, m, aliph-H). δ_{C} (151, CD₃OD) 177.2 (HO—C═O), 162.1 (5′—C═O), 155.3 (C-2′), 146.6 (C-5′), 129.7 (C-4′), 128.3 (C-3′), 54.6 (C-2), 48.4 (C-1), 31.9 (C-aliph), 28.8 (C-aliph), 23.5 (C-aliph). m/z (LCMS ES⁺) 285.5 [M+H]⁺ HRMS (ES⁺) found [M+H]⁺ 285.0557, C₁₁H₁₃N₂O₅S requires 285.0545.

### N—[±2′-(methylcarbamoyl)cyclopentyl]-5′-nitrothiophene-2-carboxamide (25)

±anti 2-(5′-nitrothiophene-2′-amido)cyclopentane-1-carboxylic acid (0.100 g, 0.35 mmol, 1.0 eq.), methylamine (2.0 M in THF, 0.3 mL, 0.62 mmol, 5.0 eq.) and triethylamine (0.05 mL, 0.25 mmol, 2.0 eq.) were dissolved in DMA (2 mL). PyBOP (0.078 g, 0.15 mmol, 1.2 eq.) was added and the reaction mixture was stirred at RT for 1.5 hrs. The reaction mixture was diluted with water (10 mL) and extracted into EtOAc (20 mL) and washed with water (3×20 mL), NaHCO₃ (3×20 mL) and brine (1×20 mL). The organic extract was dried over MgSO₄ and solvent was removed in vacuo. Trituration in EtOAc (1 mL) gave N—[+2′-(methylcarbamoyl)cyclopentyl]-5′-nitrothiophene-2-carboxamide (25) as an off white solid (0.037 g, 36%) melting point 251-252° C. νₘₐₓ (ATR) 3355 (N—H), 3274 (N—H), 1638 (C═O), 1567 (C═O), 1506, 1315 cm⁻¹. δ_{H} (700 MHz, DMSO-d₆) 8.84 (1H, d, J 7.6, 2-NH), 8.13 (1H, d, J 4.4, 4′—H), 7.82 (1H, d, J 4.4, 3′—H), 7.72 (1H, d, J 4.7, CH₃—NH), 4.35 (1H, m, 2-H), 2.66 (1H, m, 1-H), 2.55 (3H, d, J 4.7, CH₃), 1.90-1.99 (2H, m, aliph-H), 1.58-1.74 (4H, m, aliph-H). δ_{C} (176 MHz, DMSO-d₆) 173.8 (1-C═O), 159.0 (5′—C═O), 152.8 (C-2′), 146.7 (C-5′), 130.1 (C-4′), 127.3 (C-3′), 54.5 (C-2), 50.2 (C-1), 32.4 (C-aliph), 29.4 (C-aliph), 25.6 (CH₃), 23.7 (C-aliph). m/z (LCMS ES⁺) 298.2 [M+H]⁺ HRMS (ES⁺) found [M+H]⁺ 298.0871, C₁₂H₁₆ N₃O₄S requires M 298.0862. CHN found: C 48.38; H 5.15; N 13.97. C₁₂H₁₅N₃O₄S requires C 48.47 H 5.08; N 14.13.

### Ethyl 2-(5′-nitrothiophene-2′-amido)cyclopent-1-ene-1-carboxylate (26)

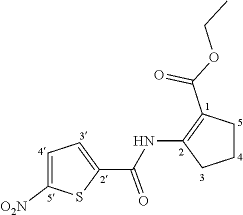 [SMILES: CCOC(=O)C1=C(NC(=O)c2ccc([N+](=O)[O-])s2)CCC1]

Following general procedure Gi, using THF (10 mL) as a reaction solvent for 44 hr, where the substituted amine was 2-aminocyclopent-1-ene-1-carboxylate (0.40 g, 2.6 mmol, 1.0 eq.) afforded, following purification by flash column chromatography (EtOAc:hexane), ethyl 2-(5′-nitrothiophene-2′-amido)cyclopent-1-ene-1-carboxylate (26) as a yellow solid (0.072 g, 9%), melting point 146-148° C. νₘₐₓ (ATR) 3090 (N—H), 1673 (C═O), 1633 (C═O), 1534, 1333 cm⁻¹. δ_{H} (599 MHz, CDCl₃) 11.49 (1H, s, NH), 7.88 (1H, d, J 4.3, 4′—H), 7.55 (1H, d, J 4.3, 3′—H), 4.26 (2H, q, J 7.1, OCH₂), 3.23 (2H, tt, J 7.7 J 2.2, 3-H), 2.53-2.59 (2H, m, 5-H), 1.93-2.01 (2H, m, 4-H), 1.33 (3H, t, J 7.1, CH₃). δ_{C} (151 MHz, CDCl₃) 168.5 (1-C═O), 157.7 (2′-C═O), 155.3 (C-5′), 154.1 (C-2), 145.0 (C-2′), 128.4 (C-4′), 127.2 (C-3′), 110.5 (C-1), 60.6 (CH—CH₃), 33.9 (C-3), 28.4 (C-5), 21.2 (C-4), 14.5 (CH₃). m/z (LCMS ES⁺) 311.3 [M+H]⁺. HRMS (ES⁺) found [M+H]⁺ 311.0711, C₁₃H₁₅N₂O₅S requires M 311.0702.

### N-(2-carbamoylphenyl)-5′-nitrothiophene-2′-carboxamide (27)

Following general procedure C, using THF (20 mL) as a reaction solvent, where the substituted amine is anthranilamide (0.21 g, 1.6 mmol, 1.0 eq.) afforded, following removal of volatiles from the filtrate and purification by flash column chromatography (EtOAc:hexane), N-(2-carbamoylphenyl)-5′-nitrothiophene-2′-carboxamide (27) as a yellow solid (0.18 g, 40%) melting point 250-251° C. νₘₐₓ (ATR) 3494 (N—H), 3396 (N—H), 3058 (N—H), 1651 (C═O), 1617 (C═O), 1527, 1340 cm⁻¹. δ_{H} (599 MHz, DMSO-d₆) 13.37 (1H, s, 1-NH), 8.49-8.52 (2H, m, 3-H & 2-CONH), 8.21 (1H, d, J 4.4, 4′—H), 7.94 (2H, dd, J 7.9, J 1.4, 6-H & 2-CONH), 7.68 (1H, d, J 4.4, 3′—H), 7.58-7.61 (1H, m, 5-H), 7.23-7.25 (1H, m, 4-H). δ_{C} (151 MHz, DMSO-d₆) 171.0 (2-C═O), 157.6 (2′—C═O), 153.5 (C-2′), 146.1 (C-5′), 139.0 (C-1), 132.8 (C-4), 130.3 (C-4′), 128.8 (C-3), 127.3 (C-3′), 123.6 (C-5), 120.3 (C-6), 119.4 (C-2). m/z (LCMS ES⁺) 292.3 [M+H]⁺. HRMS (ES⁺) found [M+H]⁺ 292.0412, C₁₂H₁₀N₃O₄S requires M 292.0392. CHN found: C 49.60; H 3.09; N 14.33. C₁₂H₉N₃O₄S requires C 49.48; H 3.11; N 14.43.

### N-[2-(methylcarbamoyl)phenyl]-5′-nitrothiophene-2′-carboxamide (28)

Following general procedure C, using DCM (8 mL) as a reaction solvent, where the substituted amine was N-[2′-(methylcarbamoyl)phenyl]-5′-nitrothiophene-2-carboxamide (0.21 g, 1.4 mmol, 1.0 eq.) afforded, following trituration with water, EtOH and DCM, N-[2-(methylcarbamoyl)phenyl]-5′-nitrothiophene-2′-carboxamide (28) as a yellow solid (0.31 g, 63%) melting point 228-229° C. νₘₐₓ (ATR) 3428 (N—H), 3118 (N—H), 1666 (C═O), 1646 (C═O), 1507, 1325 cm⁻¹. δ_{H} (700 MHz, DMSO-d₆) 13.11 (1H, s, 1-NH), 8.94 (1H, d, J 4.7, CH₃NH), 8.46 (1H, dd, J 8.5, J 1.2, Ar—H), 8.21 (1H, d, J 4.3, 4′—H), 7.85 (1H, dd, J 7.8, J 1.5, Ar—H), 7.70 (1H, d, J 4.3, 3′—H), 7.58 (1H, ddd, J 8.5, J 7.5, J 1.5, Ar—H), 7.24 (1H, ddd, J 7.8, J 7.5, J 1.2, Ar—H), 2.83 (3H, d, J 4.7, CH₃). &c (176 MHz, DMSO-d₆) 168.8 (2-C═O), 157.6 (2′-C═O), 153.4 (C-2′), 146.0 (C-5′), 138.4 (C—Ar), 132.4 (C—Ar), 130.3 (C-4′), 128.1 (C—Ar), 127.3 (C-3′), 123.7 (C—Ar), 120.4 (C—Ar), 120.1 (C—Ar), 26.4 (CH₃). m/z (LCMS ES⁺) 306.1 [M+H]⁺. HRMS (ES⁺) found [M+H]⁺ 306.0553, C₁₃H₁₂N₃O₄S requires M 306.0549. CHN found: C 51.05; H 3.59; N 13.80 C₁₃H₁₁N₃O₄S requires C 51.14; H 3.63; N 13.76.

### 2-(5′-nitrothiophene-2′-amido)benzoic acid (29) and 2-(5′-nitrothiophen-2′-yl)-4H-3,1-benzoxazin-4-one (57)

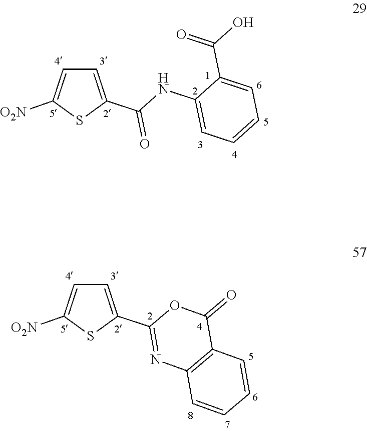 [SMILES: O=C(Nc1ccccc1C(=O)O)c1ccc([N+](=O)[O-])s1 ; O=c1oc(-c2ccc([N+](=O)[O-])s2)nc2ccccc12]

Following general procedure Gi, using THF (6 mL) as a reaction solvent, where the substituted amine was anthranilic acid (0.19 g, 1.4 mmol, 1.0 eq.) afforded, following work up with DCM and purification by flash column chromatography (DCM:MeOH), the two title products. Fractions containing 29 were purified further by recrystallisation (MeOH) to give 2-(5′-nitrothiophene-2′-amido)benzoic acid (29) as an yellow solid (0.019 g, 5%) melting point 239-240° C. νₘₐₓ (ATR) 2700-3316 (O—H), 1679 (C═O), 1663 (C═O), 1538, 1255 cm⁻¹. δ_{H} (599 MHz, CD₃OD) 8.65 (1H, dd, J 8.3 J 1.4, 6-H), 8.14 (1H, dd, J 8.2 J 1.4, 3-H), 8.01 (1H, d, J 4.3, 4′—H), 7.70 (1H, d, J 4.3, 3′—H), 7.61 (1H, ddd, J 8.2 J 7.3 J 1.4, 4-H), 7.21 (1H, ddd, J 8.3 J 7.3/1.4, 5-H). δ_{C} (151 MHz, CD₃OD) 172.1 (1-C═O), 159.8 (2′—C═O), 155.9 (C-2′), 147.1 (C-5′), 141.9 (C-2), 135.5 (C-4), 132.8 (C-3), 130.0 (C-4′), 128.1 (C-3′), 124.7 (C-5), 121.2 (C-6), 117.7 (C-1). m/z (LCMS ES⁺) 293.1 [M+H]⁺. HRMS (ES⁺) found [M+H]⁺ 293.0255, C₁₂H₉N₂O₅S requires M 292.0232. CHN found: C 49.27; H 2.83; N 9.48. C₁₂H₈N₂O₅S requires C 49.31; H 2.76; N 9.58. Fractions containing 57 were purified further by recrystallisation (DCM) to give 2-(5′-nitrothiophen-2′-yl)-4H-3,1-benzoxazin-4-one (57) as a yellow solid (0.010 g, 3%) melting point 196-197° C. νₘₐₓ (ATR) 1731-1780 (C═O, C═N), 1542, 1358 cm⁻¹. δ_{H} (599 MHz, CDCl₃) 8.25 (1H, ddd, J 8.0 J 1.3 J 0.6, 8-H), 7.93 (1H, d, J 4.3, 4′—H), 7.87 (1H, ddd, J 8.0 J 7.3 J 1.3, 7-H), 7.84 (1H, d, J 4.3, 3′—H), 7.69 (1H, ddd, J 8.0 J 1.3 J 0.6, 5-H), 7.59 (1H, ddd, J 8.0 J 7.3 J 1.3. 6-H). δ_{C} (151 MHz, CDCl₃) 158.1 (C-4), 155.3 (C-2′), 151.9 (C-2), 146.2 (6-C), 140.0 (C-5′), 137.2 (C-7), 129.7 (C-3′). 129.5 (C-6), 129.2 (C-8), 128.7 (C-4′), 127.6 (C-5), 117.2 (4-C). m/z (LCMS ES⁺) 275.2 [M+H]⁺. HRMS (ES⁺) found [M+H]⁺ 275.0158, C₁₂H₇N₂O₄S requires 275.0127.

### N-methyl-3-[(5′-nitrothiophen-2′-yl)amino]benzamide (34)

Following general procedure C, using THF (5 mL) as a reaction solvent, where the substituted amine was 3-amino-N-methyl benzamide (0.14 g, 0.94 mmol, 1.0 eq.), afforded N-methyl-3-[(5′-nitrothiophen-2′-yl)amino]benzamide (34) as a yellow solid (0.1076 g, 37%) melting point 275-277° C. νₘₐₓ (ATR) 3384 (N—H), 3258 (N—H), 1654 (C═O), 1639 (C═O), 1548, 1344 cm⁻¹. δ_{H} (700 MHz, DMSO-d₆) 10.79 (1H, s, 1-NH), 8.45-8.46 (1H, m, CH₃—NH), 8.22 (1H, d, J 4.5, 4′—H), 8.18-8.21 (1H, m, 2-H), 8.10 (1H, d, J 4.5, 3′—H), 7.93 (1H, ddd, J 8.2, J 2.3, J 1.1, 6-H), 7.61 (1H, ddd, J 7.9, J 2.5, J 1.1, 4-H), 7.46-7.48 (1H, m, 5-H), 2.79 (3H, d, J 4.5, CH₃). & (176 MHz, DMSO-d₆) 166.2 (3-C═O), 158.3 (2′—C═O), 153.5 (C-2′), 146.1 (C-5′), 138.0 (C-1), 135.3 (C-3), 130.1 (C-4′), 128.8 (C-5), 128.5 (C-3′), 123.0 (C-6), 122.7 (C-4), 119.9 (C-2), 26.3 (CH₃). m/z (LCMS ES⁺) 306.1 [M+H]⁺. HRMS (ES⁺) found [M+H]⁺ 306.0551, C₁₃H₁₁N₃O₄S requires M 306.0549. CHN found: C 51.02; H 3.77; N 13.44 C₁₃H₁₁N₃O₄S requires C 51.14; H 3.63; N 13.76.

### 5′-nitro-N-phenylthiophene-2′-carboxamide (33)

Following general procedure C, using THF (3 mL) as a reaction solvent, where the substituted amine was aniline (0.13 mL, 1.4 mmol, 1.0 eq.), afforded 5′-nitro-N-phenylthiophene-2′-carboxamide (33) as a yellow solid (0.053 g, 15%) melting point 188-189° C. νₘₐₓ (ATR) 3353 (N—H), 1656 (C═O), 1535, 1324 cm-3. δ_{H} (700 MHz, DMSO-d₆) 10.64 (1H, s, NH), 8.21 (1H, d, J 4.4, 4′—H), 8.07 (1H, d, J 4.4, 3′—H), 7.72-7.73 (2H, m, 2-H), 7.38-7.40 (2H, m, 3-H), 7.17 (1H, tt, J 7.4 J 1.2, 4-H). δ_{C} (176 MHz, DMSO-d₆) 158.2 (C═O), 153.3 (C-2′), 146.4 (C-5′), 137.9 (C-1), 130.1 (C-4′), 128.8 (C-3), 128.3 (C-3′) 124.6 (C-4), 120.6 (C-2). m/z (LCMS ES⁺) 249.2 [M+H]⁺. HRMS (ES⁺) found [M+H]⁺ 249.0357, C₁₁H₉N₂O₃S requires 249.0334. CHN found: C 53.19; H 3.29; N 11.19. C₁₁H₁₀N₂O₄S requires C 53.22; H 3.25; N 11.28.

### N-(2-methoxyphenyl)-5′-nitrothiophene-2′-carboxamide (30)

Following general procedure C, using THF (3 mL) as a reaction solvent, where the substituted amine was o-anisidine (0.25 mL, 2.2 mmol, 1.0 eq.), afforded N-(2-methoxyphenyl)-5′-nitrothiophene-2′-carboxamide (30) as a yellow solid (0.092 g, 15%) melting point 151-152° C. νₘₐₓ (ATR) 3425 (N—H), 1662 (C═O), 1530, 1336 cm⁻¹. δ_{H} (700 MHz, DMSO-d₆) 10.13 (1H, s, NH), 8.19 (1H, d, J 4.4, 4′—H), 8.05 (1H, d, J 4.4, 3′—H), 7.54 (1H, dd, J 7.7 J 1.7, 6-H), 7.26 (1H, ddd, J 8.3 J 7.4 J 1.7, 4-H), 7.13 (1H, dd, J 8.3 J 1.3, 3-H), 6.99 (1H, ddd, J 7.7 J 7.4 J 1.3, 5-H), 3.83 (3H, s, CH₃). δ_{C} (176 MHz, DMSO-d₆) 158.3 (C═O), 153.2 (C-2′), 152.5 (C-2), 146.2 (C-5′), 130.1 (C-4′), 128.2 (C-3′), 127.2 (C-4), 126.1 (C-6), 125.1 (C-1), 120.2 (C-5), 111.8 (C-3), 55.7 (CH₃). m/z (LCMS ES⁺) 279.5 [M+H]⁺. HRMS (ES⁺) found [M+H]⁺ 279.0466, C₁₂H₁₁N₂O₄S requires 279.0440. CHN found: C 51.72; H 3.64; N 9.98. C₁₂H₁₀N₂O₄S requires C 51.79; H 3.62; N 10.07

### N-(3-methoxyphenyl)-5′-nitrothiophene-2′-carboxamide (35)

Following general procedure C, using THF (2 mL) as a reaction solvent, where the substituted amine was m-anisidine (0.11 mL, 0.95 mmol, 1.0 eq.), afforded N-(3-methoxyphenyl)-5′-nitrothiophene-2′-carboxamide (35) as a yellow solid (0.024 g, 9%) melting point 180-181° C. νₘₐₓ (ATR) 3335 (N—H), 1639 (C═O), 1539, 1342 cm⁻¹. δ_{H} (700 MHz, DMSO-d₆) 10.60 (1H, s, NH), 8.21 (1H, d, J 4.4, 4′—H), 8.06 (1H, d, J 4.4, 3′—H), 7.39 (1H, m, 2-H), 7.28-7.32 (2H, m, 5-H & 6-H), 6.75 (1H, ddd, J 7.7 J 2.5 J 1.4, 4-H), 3.76 (3H, s, CH₃). δ_{C} (176 MHz, DMSO-d₆) 159.5 (C-3), 158.2 (C═O), 153.4 (C-2′), 146.3 (C-5′), 139.1 (C-1), 130.1 (C-4′), 129.7 (C-5), 128.3 (C-3′), 112.8 (C-6), 110.2 (C-4), 106.3 (C-2), 55.1 (CH₃). m/z (LCMS ES⁺) 279.0 [M+H]⁺. HRMS (ES⁺) found [M+H]⁺ 279.0471, C₁₂H₁₁N₂O₄S requires 279.0440. CHN found: C 51.71; H 3.57; N 9.96. C₁₂H₁₀N₂O₄S requires C 51.79; H 3.62; N 10.07.

### N-(4-methoxyphenyl)-5′-nitrothiophene-2′-carboxamide (36)

Following general procedure C, using THF (2 mL) as a reaction solvent, where the substituted amine was p-anisidine (0.12 g. 0.95 mmol, 1.0 eq.), afforded N-(4-methoxyphenyl)-5′-nitrothiophene-2′-carboxamide (36) as a yellow solid (0.035 g, 13%) melting point 187-188° C. νₘₐₓ (ATR) 3341 (N—H), 1634 (C═O), 1528, 1327 cm⁻¹. δ_{H} (599 MHz, DMSO-d₆) 10.55 (1H, s, NH), 8.20 (1H, d, J 4.5, 4′—H), 8.03 (1H, d, J 4.5, 3′—H), 7.62-7.64 (2H, m, 2-H), 6.95-6.97 (2H, m, 3-H), 3.75 (3H, s, CH₃). δ_{C} (151 MHz, DMSO-d₆) 157.8 (C═O), 156.2 (C-4), 153.1 (C-2′), 146.7 (C-5′), 130.9 (C-1), 130.1 (C-4′), 128.0 (C-3′), 122.3 (C-2), 114.0 (C-3), 55.2 (CH₃). m/z (LCMS ES⁺) 279.2 [M+H]⁺. HRMS (ES⁺) found [M+H]⁺ 279.0441, C₁₂H₁₁N₂O₄S requires 279.0440. CHN found: C 51.27; H 3.62; N 9.91. C₁₂H₁₀N₂O₄S requires C 51.79; H 3.62; N 10.07.

### 5′-nitro-N-[2-(trifluoromethyl)phenyl]thiophene-2′-carboxamide (31)

Following general procedure Gi, using DCM (4 mL) as a reaction solvent, where the substituted amine was 2-trifluoromethyl aniline (0.16 mL, 1.24 mmol, 1.0 eq.) afforded, following work up with DCM and purification by flash column chromatography (EtOAc:hexane), 5-amino-N-[2-(trifluoromethyl)phenyl]thiophene-2-carboxamide (31) as a yellow solid (0.16 g, 41%), melting point 129-130° C. νₘₐₓ (ATR) 3275 (N—H), 1647 (C═O), 1545, 1321 cm⁻¹. δ_{H} (700 MHz, CDCl₃) 8.30 (1H, d, J 8.2, 6-H), 8.10 (1H, s, NH), 7.92 (1H, d, J 4.3, 4′—H), 7.69 (1H, d, J 7.9, 3-H), 7.64 (1H, d, J 8.2 J 7.7, 5-H), 7.46 (1H, d, J 4.3, 3′—H), 7.34 (1H, dd, J 7.9 J 7.7, 4-H). δ_{C} (176 MHz, CDCl₃) 158.2 (C═O), 155.3 (C-2′), 144.4 (C-5′), 134.3 (C-1), 133.4 (C-5), 128.3 (C-4′), 126.6 (q, J 5.2, C-3), 126.5 (C-3′), 125.7 (C-4), 124.5 (C-6), 124.3 (d, J 273.0, CF₃), 120.6 (d, J 29.0, C-2). δ_{F} (376 MHz, CDCl₃) −60.1 (CF₃). m/z (LCMS ES⁺) 317.2 [M+H]⁺. HRMS (ES⁺) found [M+H]⁺ 317.0210, C₁₂H₇N₂O₃F₃S requires M 317.0208. CHN found: C 45.70; H 2.24; N 8.78. C₁₂H₇N₂O₃F₃S requires C 45.57; H 2.23; N 8.86.

### 5′-nitro-N-[3-(trifluoromethyl)phenyl]thiophene-2′-carboxamide (37)

Following general procedure C, using DCM (4 mL) as a reaction solvent, where the substituted amine was 3-trifluoromethyl aniline (0.16 mL, 1.24 mmol, 1.0 eq.), afforded 5-amino-N-[3-(trifluoromethyl)phenyl]thiophene-2-carboxamide (37) as a yellow solid (0.37 g, 94%), melting point 178-179° C. νₘₐₓ (ATR) 3404 (N—H), 1683 (C═O), 1555, 1316 cm⁻¹. δ_{H} (700 MHz, DMSO-d₆) 10.90 (1H, s, NH), 8.22 (1H, d, J 4.4, 4′—H), 8.16 (1H, d, J 2.0, 2-H), 8.07 (1H, d, J 4.4, 3′—H), 8.01 (1H, ddd, J 8.1 J 2.0 J 1.0, 4-H), 7.64 (1H, dd, J 8.1 J 7.8, 5-H), 7.52 (1H, ddd, J 8.1 J 2.0 J 1.0, 6-H). δ_{C} (176 MHz, DMSO-d₆) 158.7 (C═O), 153.6 (C-2′), 145.5 (C-5′), 138.8 (C-1), 130.2 (C-5), 130.1 (C-4′), 129.5 (q, J 31.2, C-3), 128.8 (C-3′), 124.0 (q, J 274.3, CF₃), 124.0 (C-6), 120.9 (d, J 3.8, C-4), 116.6 (q, J 4.1, C-2). OF (376 MHz, CDCl₃) −62.8 (CF₃). m/z (LCMS ES⁺) 317.1 [M+H]⁺. HRMS (ES⁺) found [M+H]⁺ 317.0216, C₁₂H₈N₂O₃F₃S requires M 317.0208. CHN found: C 45.57; H 2.22; N 8.89. C₁₂H₇N₂O₃F₃S requires C 45.57; H 2.23; N 8.86

### N-(2-acetylphenyl)-5′-nitrothiophene-2′-carboxamide (32)

Following general procedure C, using DCM (3 mL) as a reaction solvent for 6 hrs, where the substituted amine was 2-aminoacetophenone (0.20 g, 1.5 mmol, 1.0 eq.), afforded N-(2-acetylphenyl)-5′-nitrothiophene-2′-carboxamide (32) as a yellow solid (0.268 g, 62%) melting point 215-216° C. νₘₐₓ (ATR) 3106 (N—H), 1664 (C═O), 1644 (C═O), 1518, 1316 cm⁻¹. δ_{H} (599 MHz, DMSO-d₆) 12.30 (1H, s, NH), 8.32 (1H, dd, J 8.3 J 1.2, 6-H), 8.23 (1H, d, J 4.4, 4′—H), 8.09 (1H, dd, J 8.2 J 1.5, 3-H), 7.84 (1H, d, J 4.4, 3′—H), 7.68-7.71 (1H, m, 5-H), 7.33-7.36 (1H, m, 4-H), 2.68 (3H, d, J 0.9, CH₃). δ_{C} (151 MHz, DMSO-d₆) 203.1 (2-C═O), 158.3 (2′-C═O), 153.7 (C-2′), 145.4 (C-5′), 137.7 (C-1), 134.4 (C-5), 131.8 (C-3), 130.3 (C-4′), 127.9 (C-3′), 125.3 (C-2), 124.4 (C-4), 121.3 (C-6), 28.7 (CH₃). m/z (LCMS ES⁺) 291.2 [M+H]⁺. HRMS (ES⁺) found [M−H]⁻ 289.0262, C₁₃H₉N₂O₄S requires 289.0283. CHN found: C 53.75; H 3.43; N 9.73. C₁₃H₁₀N₂O₄S requires C 53.79; H 3.47; N 9.66.

### N-[2-(dimethylcarbamoyl)phenyl]-5′-nitrothiophene-2′-carboxamide (56)

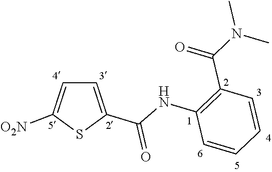 [SMILES: CN(C)C(=O)c1ccccc1NC(=O)c1ccc([N+](=O)[O-])s1]

Following general procedure Gii, using THF (5 mL) as a reaction solvent for 6 hrs, where the substituted amine was 2-amino-N-dimethyl benzamide (0.26 g, 1.6 mmol, 1.0 eq.) afforded, following filtration and trituration of the crude solid and purification of the worked up filtrate by flash column chromatography (EtOAc:hexane), N-[2-(dimethylcarbamoyl)phenyl]-5′-nitrothiophene-2′-carboxamide (56) as a pale yellow solid (0.12 g, 24%) melting point 214-216° C. νₘₐₓ (ATR) 3230 (N—H), 1659 (C═O), 1618 (C═O), 1546, 1360 cm⁻¹. δ_{H} (599 MHz, DMSO-d₆) 10.68 (1H, s, 1-NH), 8.19 (1H, d, J 4.4, 4′—H), 7.92 (1H, d, J 4.4, 3′—H), 7.46-7.53 (2H, m, 3-H & 4-H), 7.38 (1H, dd, J 7.5 J 1.6, 6-H), 7.33 (1H, ddd, J 7.5, J 1.4, 5-H), 2.91 (3H, s, CH₃), 2.87 (3H, s, CH₃). δ_{C} (151 MHz, DMSO-d₆) 168.5 (2-C═O), 158.5 (C-2′), 153.3 (2′—C═O), 145.6 (C-5′), 133.8 (C-1), 131.6 (C-2), 130.2 (C-4′), 129.6 (C-3 or C-4), 128.3 (C-3′), 127.8 (C-6), 125.9 (C-3 or C-4 and C-5), 38.8 & 34.6 (CH₃). m/z (LCMS ES⁺) 320.3 [M+H]⁺. HRMS (ES⁺) found [M+H]⁺ 320.0713, C₁₄H₁₄N₃O₄S requires M 320.0705.

### 5′-nitro-N-[2-(tridecylcarbamoyl)phenyl]thiophene-2′-carboxamide (39)

2-(5′-nitrothiophen-2′-yl)-4H-3,1-benzoxazin-4-one (0.02 g, 0.07 mmol, 1.0 eq.) was dissolved in dry chloroform (0.5 mL). Tridecylamine (0.048 g, 0.24 mmol, 3.2 eq.) in dry chloroform (0.5 mL) was added dropwise at 0° C. The reaction mixture was stirred at RT for 1 hour. Volatiles were removed in vacuo before the crude solid was triturated in hexane to give 5′-nitro-N-[2-(tridecylcarbamoyl)phenyl]thiophene-2′-carboxamide (39) as a yellow solid (0.027 g, 82%), melting point 112-113° C. νₘₐₓ (ATR) 3336 (N—H), 1681 (C═O), 1626 (C═O), 1522, 1332 cm⁻¹. δ_{H} (599 MHz, CDCl₃) 12.72 (1H, s, 1-NH), 8.70 (1H, d, J 8.5, 6-H), 7.90 (1H, d, J 4.3, 4′—H), 7.65 (1H, d, J 4.3, 3′—H), 7.51-7.56 (2H, m, 3-H & 5-H), 7.16 (1H, d, J 7.4, 4-H), 6.36 (1H, s, 2-CONH), 3.45-3.49 (2H, m, NH—CH₂), 1.62-1.67 (2H, m, NHCH₂-CH₂), 1.25-1.41 (20H, m, CH₂), 0.88 (3H, t, J 7.0, CH₃). δ_{C} (151 MHz, CDCl₃) 169.2 (2-C═O), 158.4 (2′-C═O), 154.8 (C-2′), 146.7 (C-5′), 139.4 (C-1), 133.2 (Ar—C), 128.6 (C-4′), 126.5 (C-3′ & Ar—C), 123.8 (C-4), 121.6 (C-6), 119.9 (C-2), 40.4 (CH₂-NH), 32.1 (CH₂), 29.4-29.8 (CH₂), 27.1 (CH₂), 22.8 (CH₂), 14.3 (CH₃). m/z (LCMS ES⁺) 474.3 [M+H]⁺. HRMS (ES⁺) found [M+H]⁺ 474.2426, C₂₅H₃₆N₃O₄S requires 474.2427. CHN found: C 63.57; H 7.56; N 8.40. C₂₅H35N₃O₄S requires C 63.40; H 7.45; N 8.87.

### 5′-nitro-N-{[(propan-2-yl) carbamoyl]-2-phenyl}thiophene-2′-carboxamide (38)

2-(5′-nitrothiophen-2′-yl)-4H-3,1-benzoxazin-4-one (0.013 g, 0.05 mmol, 1.0 eq.) was dissolved in dry chloroform (0.5 mL). Isopropylamine (0.02 mL, 0.22 mmol, 4.4 eq.) in dry chloroform (0.5 mL) was added dropwise at 0° C. The reaction mixture was stirred at RT for 3 hrs. Volatiles were removed in vacuo to give 5′-nitro-N-{[(propan-2-yl) carbamoyl]-2-phenyl}thiophene-2′-carboxamide (38) as a yellow solid (0.016 g, 100%), melting point 234° C. νₘₐₓ (ATR) 3305 (N—H), 1679 (C═O), 1627 (C═O), 1513, 1340 cm⁻¹. δ_{H} (599 MHz, CDCl₃) 12.74 (1H, s, 2-NH), 8.70 (1H, dd, J 8.4 J 1.0, 3-H), 7.90 (1H, dd, J 4.3 J 0.9, 4′—H), 7.65 (1H, dd, J 4.3 J 0.9, 3′—H), 7.50-7.56 (2H, m, 4-H & 6-H), 7.16 (1H, ddd, J 8.6 J 7.5 J 1.1, 5-H), 6.16 (1H, d, J 7.2, CH—NH), 4.27-4.34 (1H, m, CH—NH), 1.31 (dd, J 6.6, J 0.9, CH₃). δ_{C} (151 MHz, CDCl₃) 168.4 (1-C═O), 158.4 (2′-C═O), 154.8 (C-2′), 146.8 (C-5′), 139.5 (C-2), 133.2 (C-4), 128.6 (C-4′), 126.5 (C-3′ & C-6), 123.8 (C-5), 121.6 (C-3), 120.0 (C-1), 42.4 (CH—CH₃), 22.8 (CH₃). m/z (LCMS ES⁺) 334.8 [M+H]⁺. HRMS (ES⁻) found [M−H]⁻ 332.0700, C₁₅H₁₄N₃O₄S requires 332.0705.

### N-[3-methyl-2-(methylcarbamoyl)phenyl]-5′-nitrothiophene-2′-carboxamide (40)

Following general procedure C, using DCM (7 mL) as a reaction solvent, where the substituted amine was amino-3-methyl-N-methyl-2-benzamide (0.59 g, 3.59 mmol, 1.0 eq.), afforded N-[3-methyl-2-(methylcarbamoyl)phenyl]-5′-nitrothiophene-2′-carboxamide (40) as a yellow solid (0.39 g, 34%) melting point 211-212° C. νₘₐₓ (ATR) 3319 (N—H), 3268 (N—H), 1630 (C═O), 1608 (C═O), 1547, 1302 cm⁻¹. δ_{H} (700 MHz, DMSO-d₆) 10.39 (1H, s, 1-NH), 8.18 (1H, d, J 4.4, 4′—H), 8.14 (1H, m, CH₃—NH), 7.87 (1H, d, J 4.4, 3′—H), 7.43 (1H, d, J 7.9, 6-H), 7.34 (1H, dd, J 7.9 J 7.7, 5-H), 7.17 (1H, d, J 7.7, 4-H), 2.72 (3H, d, J 4.6, NHCH₃), 2.30 (3H, s, Ar—CH₃). δ_{C} (176 MHz, DMSO-d₆) 167.2 (2-C═O), 158.6 (2′—C═O), 153.3 (C-2′), 145.8 (C-5′), 135.5 (C-1), 133.5 (C-3), 133.4 (C-2), 130.2 (C-4′), 128.8 (C-5), 128.1 (C-3′ & C-4), 123.5 (C-6), 25.9 (CH₃NH), 19.5 (CH₃Ar). m/z (LCMS ES⁺) 320.3 [M+H]⁺. HRMS (ES⁺) found [M+H]⁺ 320.0703, C₁₄H₁₄N₃O₄S requires 320.0705. CHN found: C 52.41; H 3.99; N 13.08. C₁₄H₁₃N₃O₄S requires C 52.66; H 4.10; N 13.16.

### N-[4-methoxy-2-(methylcarbamoyl)phenyl]-5′-nitrothiophene-2′-carboxamide (41)

Following general procedure C, using DCM (1 mL) as a reaction solvent, where the substituted amine was amino-4-methoxy-N-methyl-2-benzamide (0.032 g, 0.18 mmol, 1.0 eq.), afforded N-[4-methoxy-2-(methylcarbamoyl)phenyl]-5′-nitrothiophene-2′-carboxamide (41) as a yellow solid (0.040 g, 68%) melting point 281-282° C. νₘₐₓ (ATR) 3360 (N—H), 3085 (N—H), 1664 (C═O), 1597 (C═O), 1525, 1334 cm⁻¹. δ_{H} (700 MHz, DMSO-do) 12.69 (1H, s, 1-NH), 8.91 (1H, d, J 4.7, CH₃NH), 8.34 (1H, d, J 9.1, 6-H), 8.21 (1H, d, J 4.4, 4′—H), 7.70 (1H, d, J 4.4, 3′—H), 7.39 (1H, d, J 2.9, 3-H), 7.18 (1H, dd, J 9.1 J 2.9, 5-H), 3.82 (3H, s, O—CH₃), 2.83 (3H, d, J 4.7, NHCH₃). δ_{C} (176 MHz, DMSO-d₆) 168.4 (2-C═O), 157.2 (2′-C═O), 155.2 (C-4), 153.2 (C-2′), 146.3 (C-5′), 131.3 (C-1), 130.4 (C-4′), 127.0 (C-3′), 122.4 (C-6), 122.2 (C-2), 117.8 (C-5), 113.1 (C-3), 55.6 (O—CH₃), 26.4 (NH—CH₃). m/z (LCMS ES⁺) 336.3 [M+H]⁺. HRMS (ES⁺) found [M+H]⁺ 336.0657, C₁₄H₁₃N₃O₅S requires 336.0654. CHN found: C 49.22; H 3.81; N 12.21. C₁₄H₁₄N₃O₅S requires C 50.14; H 3.91; N 12.54.

### N-[4-methyl-2-(methylcarbamoyl)phenyl]-5′-nitrothiophene-2′-carboxamide (42)

Following general procedure C, using DCM (4 mL) as a reaction solvent, where the substituted amine was amino-4-methyl-N-methyl-2-benzamide (0.16 g, 0.97 mmol, 1.0 eq.), afforded N-[4-methyl-2-(methylcarbamoyl)phenyl]-5′-nitrothiophene-2′-carboxamide (42) as a yellow solid (0.22 g, 70%) melting point 272-274° C. νₘₐₓ (ATR) 3436 (N—H), 3115 (N—H), 1671 (C═O) 1643 (C═O), 1525, 1329 cm⁻¹. δ_{H} (700 MHz, DMSO-d₆) 12.98 (1H, s, 1-NH), 8.89 (1H, s, NHCH₃), 8.34 (1H, d, J 8.4, 6-H), 8.20 (1H, d, J 4.3, 4′—H), 7.67-7.69 (2H, m, 3′-H & 3-H), 7.38 (1H, dd, J 8.4 J 2.0, 5-H), 2.82 (3H, d, J 4.5, NH—CH₃), 2.33 (3H, s, Ar—CH₃). δ_{C} (176 MHz, DMSO-d₆) 168.8 (2-C═O), 157.3 (2′—C═O), 153.3 (C-2′), 146.2 (C-5′), 136.0 (C-1), 133.0 (C-4), 132.8 (C-5), 130.3 (C-4′), 128.4 (C-3), 127.1 (C-3′), 120.4 (C-6), 120.1 (C-2), 26.4 (CH₃—NH), 20.4 (CH₃-Ar). m/z (LCMS ES⁺) 320.3 [M+H]⁺. HRMS (ES⁺) found [M+H]⁺ 320.0703, C₁₄H₁₄N₃O₄S requires 320.0705. CHN found: C 52.48; H 4.05; N 12.97. C₁₄H₁₃N₃O₄S requires C 52.66; H 4.10; N 13.16.

### N-[5-methoxy-2-(methylcarbamoyl)phenyl]-5′-nitrothiophene-2′-carboxamide (43)

Following general procedure C, using DCM (1 mL) as a reaction solvent, where the substituted amine was amino-5-methoxy-N-methyl-2-benzamide (0.039 g, 0.22 mmol, 1.0 eq.), afforded N-[5-methoxy-2-(methylcarbamoyl)phenyl]-5′-nitrothiophene-2′-carboxamide (43) as a yellow solid (0.041 g, 57%) melting point 230-231° C. νₘₐₓ (ATR) 3210 (N—H), 1764 (C═O), 1705 (C═O) 1592, 1348 cm⁻¹. δ_{H} (700 MHz, DMSO-d₆) 13.70 (1H, s, 1-NH), 8.82 (1H, d, J 4.6, NHCH₃), 8.22 (1H, d, J 4.4, 4′—H), 8.18 (1H, d, J 2.7, 6-H), 7.87 (1H, d, J 8.9, 3-H), 7.69 (1H, d, J 4.4, 3′—H), 6.82 (1H, d, J 8.9 J 2.7, 4-H), 3.83 (3H, s, O—CH₃), 2.82 (3H, d, J 4.6, NHCH₃). δ_{C} (176 MHz, DMSO-d₆) 168.8 (2-C═O), 162.1 (C-5), 157.7 (2′—C═O), 153.5 (C-2′), 146.0 (C-5′), 140.7 (C-1), 130.4 (C-4′), 129.7 (C-3), 127.2 (C-3′), 111.6 (C-2), 109.2 (C-4), 105.1 (C-6), 55.5 (O—CH₃), 26.3 (NH—CH₃). m/z (LCMS ES⁺) 336.3 [M+H]⁺. HRMS (ES⁺) found [M+H]⁺ 336.0667, C₁₄H₁₄N₃O₅S requires 336.0654. CHN found: C 49.61; H 3.88; N 12.31. C₁₄H₁₃N₃O₅S requires C 50.14; H 3.91; N 12.54.

### N-[5-methyl-2-(methylcarbamoyl)phenyl]-5′-nitrothiophene-2′-carboxamide (44)

Following general procedure C, using DCM (6 mL) as a reaction solvent, where the substituted amine was amino-5-methyl-N-methyl-2-benzamide (0.21 g, 1.28 mmol, 1.0 eq.), afforded N-[5-methyl-2-(methylcarbamoyl)phenyl]-5′-nitrothiophene-2′-carboxamide (44) as a yellow solid (0.26 g, 64%) melting point 206-207° C. νₘₐₓ (ATR) 3439 (N—H) 3118 (N—H), 1653 (C═O), 1586 (C═O), 1529, 1335 cm⁻¹. δ_{H} (599 MHz, DMSO-d₆) 13.29 (1H, s, 1-NH), 8.88-8.89 (1H, m, CH₃—NH), 8.35 (1H, d, J 1.8, 6-H), 8.21 (1H, d, J 4.4, 4′—H), 7.77 (1H, d, J 8.0, 3-H), 7.69 (1H, d, J 4.4, 3′—H). 7.06 (1H, ddd, J 8.0 J 1.8 J 0.9, 4-H) 2.82 (3H, d, J 4.5, NHCH₃), 2.36 (3H, s, Ar—CH₃). δ_{C} (151 MHz, DMSO-d₆) 168.9 (2-C═O), 157.5 (2′—C═O), 153.4 (C-2′), 146.2 (C-5′), 142.7 (C-5), 138.7 (C-1), 130.4 (C-4′), 128.0 (C-3), 127.2 (C-3′), 124.3 (C-4), 120.6 (C-6), 116.9 (C-2), 26.4 (NH—CH₃), 21.3 (Ar—CH₃). m/z (LCMS ES⁺) 320.3 [M+H]⁺. HRMS (ES⁺) found [M+H]⁺ 320.0697, C₁₄H₁₄N₃O₄S requires 320.0705. CHN found: C 52.76; H 4.12; N 13.06. C₁₄H₁₃N₃O₄S requires C 52.66; H 4.10; N 13.16.

### N-[2-(methylcarbamoyl)-5-(trifluoromethyl)phenyl]-5′-nitrothiophene-2′-carboxamide (45)

Following general procedure Gi, using THF (6 mL) as a reaction solvent, where the substituted amine was amino-5-trifluoromethyl-N-methyl-2-benzamide (0.20 g, 0.92 mmol, 1.0 eq.) afforded, following work up in DCM and recrystallisation from chloroform: hexane, N-[2-(methylcarbamoyl)-5-(trifluoromethyl)phenyl]-5′-nitrothiophene-2′-carboxamide (45) as a yellow solid (0.060 g, 18%) melting point 228° C. νₘₐₓ (ATR) 3384 (N—H), 3123 (N—H), 1683 (C═O), 1650 (C═O), 1540, 1338 cm⁻¹. δ_{H} (400 MHz, DMSO-d₆) 12.99 (1H, s, 1-NH), 9.16 (1H, s, NH—CH₃), 8.79 (1H d, J 5.0 6-H), 8.24 (1H, d, J 4.4, 4′—H), 8.05 (1H, d. J 8.3, 3-H), 7.74 (1H, d. J 4.4, 3′—H), 7.65 (1H, m, 4-H), 2.85 (3H, d, J 4.4, CH₃). δ_{C} (101 MHz, DMSO-d₆) 167.6 (2-C═O), 158.7 (2′-C═O), 153.8 (C-2′), 145.2 (C-5′), 138.7 (C-1), 131.8 (C-5), 130.4 (C-4′), 129.5 (C-3), 127.9 (C-3′), 123.8 (C-2), 123.5 (q, J 272.7, CF₃), 120.4 (C-4), 117.1 (C-6), 26.6 (CH₃). 8F (376 MHz, DMSO-d₆) −61.9 (CF₃). m/z (LCMS ES⁺) 374.2 [M+H]⁺. HRMS (ES⁺) found [M+H]⁺ 374.0440, C₁₄H₁₁N₃O₄F₃S requires M 374.0422. CHN found: C 44.83; H 2.77; N 11.08. C₁₄H₁₀N₃O₄F₃S requires C 45.04; H 2.70; N 11.26.

### N-[5-bromo2-(methylcarbamoyl)phenyl]-5′-nitrothiophene-2′-carboxamide (46)

Followed general procedure C, using DCM (5 mL) as a reaction solvent, where the substituted amine was amino-5-bromo-N-methyl-2-benzamide (1.03 g, 4.5 mmol, 1.0 eq.) afforded, following recrystallisation (pyridine/hexane), N-[5-bromo2-(methylcarbamoyl)phenyl]-5′-nitrothiophene-2′-carboxamide (46) (0.39 g, 23%) as a yellow solid, melting point 238° C. δ_{H} (700 MHz, DMSO-d₆) 13.22 (1H, s, 1-NH), 9.03 (1H, d, J 4.7, CH₃—NH), 8.70 (1H, d, J 2.1, 6-H), 8.22 (1H, d, J 4.4, 4′—H), 7.81 (1H, d, J 8.5, 3-H), 7.70 (1H, d, J 4.4, 3′—H), 7.48 (1H, dd, J 8.5 J 2.1, 4-H), 2.83 (3H, d, J 4.7, CH₃). δ_{C} (176 MHz, DMSO-d₆) 168.1 (2-C═O), 157.9 (2′-C═O), 153.7 (C-2′), 145.3 (C-5′), 139.7 (C-1), 130.3 (C-4′), 129.9 (C-3), 127.7 (C-3′), 126.4 (C-4), 125.5 (C-5), 122.7 (C-6), 118.9 (C-2), 26.4 (CH₃). m/z (LCMS ES⁺) 384.2 [M(⁷⁹Br)+H]⁺& 386.2 [M(⁸¹Br)+H]⁺. HRMS (ES⁺) found [M+H]⁺ 383.9648, C₁₃H₁₁N₂O₄SBr requires 383.9654. CHN found: C 40.51; H 2.61; N 10.92. C₁₄H₁₄N₃O₅S requires C 40.63; H 2.62; N 10.94.

### N-methyl-2-(N-methyl-5′-nitrothiophene-2′-amido)thiophene-3-carboxamide (48)

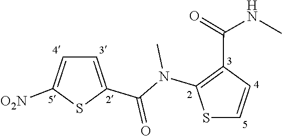 [SMILES: CNC(=O)c1ccsc1N(C)C(=O)c1ccc([N+](=O)[O-])s1]

Followed general procedure Gi, using DCM (1.4 mL) as a reaction solvent for 1 hr, where the substituted amine was N-methyl-2-(methylamino)thiophene-3-carboxamide (0.12 g, 0.70 mmol, 1.0 eq.) afforded, following work up in DCM and purification by flash column chromatography (EtOAc:hexane), N-methyl-2-(N-methyl-5′-nitrothiophene-2′-amido)thiophene-3-carboxamide (48) as a pale yellow solid (0.13 g, 59%) melting point 101-102° C. νₘₐₓ (ATR) 3323 (N—H), 1630 (C═O), 1504, 1330 cm⁻¹. δ_{H} (599 MHz, CDC)₃) 7.64 (1H, d, J 4.4, 4′—H), 7.31 (1H, d, J 5.8, 5-H), 7.22 (1H, d, J 5.8, 4-H), 7.12 (1H, d, J 4.4, 3′—H), 6.15 (1H, s, NH), 3.45 (3H, s, 2-NCH₃), 2.88 (3H, d, J 4.9, NH—CH₃). δ_{C} (151 MHz, CDCl₃) 162.0 (3-C═O), 161.6 (2′-C═O), 154.6 (C-2′), 146.8 (C-2), 141.8 (C-5′), 132.7 (C-3), 131.1 (C-3′), 127.3 (C-4′), 126.0 (C-4), 125.2 (C-5), 40.2 (2-NCH₃), 26.7 (NH—CH₃). m/z (LCMS ES⁺) 326.2 [M+H]⁺. HRMS (ES⁺) found [M+H]⁺ 326.0262, C₁₂H₁₂N₃O₄S₂ requires 326.0269.

### 1-(5′-nitrothiophene-2′-carbonyl)-1,2,3,4-tetrahydroquinoline (49)

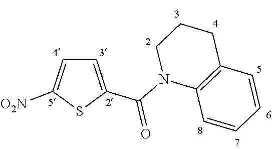 [SMILES: O=C(c1ccc([N+](=O)[O-])s1)N1CCCc2ccccc21]

Following general procedure Gi, using DCM (4 mL) as a reaction solvent, where the substituted amine was 1,2,3,4-tetrahydroquinoline (0.19 mL, 1.50 mmol, 1.0 eq.) afforded, following work up with 1M NaOH, 1-(5′-nitrothiophene-2′-carbonyl)-1,2,3,4-tetrahydroquinoline (208) as a yellow solid (0.34 g, 79%) melting point 97-98° C. νₘₐₓ (ATR) 1632 (C═O), 1536, 1339 cm⁻¹. δ_{H} (700 MHz, CDCl₃) 7.62 (1H, d, J 4.3, 4′—H), 7.24 (1H, dd, J 7.5 J 1.2, 5-H), 7.16 (1H, ddd, J 8.0 J 7.8 J 1.2, 7-H), 7.03 (1H, dd, J 7.8 J 7.5, 6-H), 6.90 (1H, d, J 8.0, 8-H), 6.81 (1H, d, J 4.3, 3′—H), 3.92 (2H, t, J 6.8, 2-H₂), 2.81 (2H, t, J 6.6, 4-H₂), 2.07 (2H, dt, J 6.8 J 6.6, 3-H₂). δ_{C} (176 MHz, CDCl₃) 161.2 (C═O), 153.8 (C-2′), 144.8 (C-5′), 138.3 (1-C(Ar)) 133.7 (4-C(Ar)), 129.7 (C-3′), 128.8 (C-5), 127.3 (C-4′), 126.6 (C-7), 126.5 (C-6), 125.6 (C-8), 44.8 (C-2), 26.8 (C-4), 24.2 (C-3). m/z (LCMS ES⁺) 289.2 [M+H]⁺. HRMS (ES⁺) found [M+H]⁺ 289.0657, C₁₄H₁₃N₂O₃S requires M 289.0647. CHN found: C 58.16; H 4.20; N 9.57. C₁₂H₇N₂O₃F₃S requires C 58.32; H 4.20; N 9.72.

### N-(2-carbamoylbenzyl)-5′-nitrothiophene-2′-carboxamide (47)

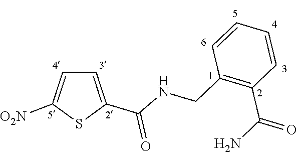 [SMILES: NC(=O)c1ccccc1CNC(=O)c1ccc([N+](=O)[O-])s1]

Following general procedure Gi, using THF (1 mL) as a reaction solvent and triethylamine (3.5 eq.) for 1 hr, where the substituted amine was 2-aminomethylbenzamide hydrochloride (54 mg, 0.29 mmol, 1.0 eq.), afforded, without further purification, N-(2-carbamoylbenzyl)-5′-nitrothiophene-2′-carboxamide (47) as a yellow solid (18 mg, 20%) melting point 185-186° C. νₘₐₓ (ATR) 3080 (NH), 1654 (C═O), 1621 (C═O), 1509, 1334 cm⁻¹. δ_{H} (599 MHz, CD₃OD) 7.96 (1H, d, J 4.9, 4′—H), 7.65 (1H, d, J 4.9, 3′—H), 7.55 (1H, d, J 8.5, 3-H), 7.45-7.47 (2H, m, 5-H & 6-H), 7.34-7.38 (1H, m, 4-H), 4.72 (2H, s, 1-CH₂). δ_{C} (151 MHz, CD₃OD) 174.7 (2-C═O), 162.1 (2′-C═O), 155.5 (C-5′), 146.3 (C-2′), 137.4 (C-1), 136.6 (C-2), 131.7 & 130.3 (C-5 & C-6), 129.8 (C-4′), 128.9 (C-3), 128.7 (C-4), 128.2 (C-3′), 42.9 (1-CH₂). m/z (LCMS ES⁺) 306.3 [M+H]⁺. HRMS (ES⁺) found [M+H]⁺ 306.0554, C₁₃H₁₂N₃O₄S requires M 306.0549.

### N-methyl-2-[(E)-[(5′-nitrothiophen-2′-yl)methylidene]amino]thiophene-3-carboxamide (50)

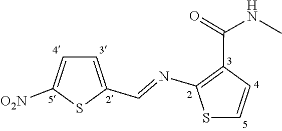 [SMILES: CNC(=O)c1ccsc1/N=C/c1ccc([N+](=O)[O-])s1]

5-nitrothiophene-2-carboxaldehyde (0.22 g, 1.4 mmol, 1.0 eq.) was dissolved in dry MeOH (2 mL) and added to a solution of 2-amino-N-methylthiophene-3-carboxamide (0.27 g, 1.7 mmol, 1.2 eq.) and acetic acid (0.01 mL, 0.14 mmol, 0.1 eq.) in dry MeOH (2 mL). The reaction was stirred at RT for 4 hrs. The reaction mixture was filtered to give an orange solid, which was purified by flash column chromatography (CHCl₃:MeOH) to give N-methyl-2-[(E)-[(5′-nitrothiophen-2′-yl)methylidene]amino]thiophene-3-carboxamide (50) as an orange solid of 80% purity (0.076 g, 14%) melting point 183-187° C. νₘₐₓ (ATR) 3310 (N—H), 1647 (C═N), 1582 (C═O), 1552, 1334 cm⁻¹. TH (700 MHz, DMSO-d₆) 8.90 (1H, s, CHN), 8.21 (2H, m, 4′-H & NH), 7.84 (1H, d, J 4.4, 3′—H), 7.48 (1H, d, J 5.6, 5-H), 7:35 (1H, d, J 5.6, 4-H), 2.88 (3H, d, J 4.8, CH₃). δ_{C} (176 MHz, DMSO-d₆) 162.1 (C═O), 153.2 (C-5′), 152.0 (2′-CHN), 151.0 (C-2), 147.0 (C-2′), 133.3 (C-3′), 132.5 (C-3), 130.6 (C-4′), 128.9 (C-4), 123.2 (C-5), 26.0 (CH₃). m/z (LCMS ES⁺) 296.1 [M+H]⁺. HRMS (ES⁺) found [M+H]⁺ 296.0192, C₁₁H₁₀N₃O₃S₂ requires M 296.0164. CHN found: C 44.76; H 3.39; N 13.42. C₁₇H₁₉N₃O₄S₂ requires C 44.73; H 3.07; N 14.23.

### N-[5-tert-butyl-2-(methylcarbamoyl)phenyl]-5′-nitrothiophene-2′-carboxamide (55)

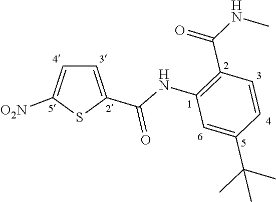 [SMILES: CNC(=O)c1ccc(C(C)(C)C)cc1NC(=O)c1ccc([N+](=O)[O-])s1]

Following general procedure C, using DCM (2 mL) as a reaction solvent for 3 hrs, where the substituted amine was 2-amino-4-tert-butyl-N-methylbenzamide (0.11 g, 0.53, 1.0 eq.) afforded, after dilution with DCM and washing with water and brine, removal of volatiles, trituration with water and recrystallisation from CHCl₃:hexane, N-[5-tert-butyl-2-(methylcarbamoyl)phenyl]-5′-nitrothiophene-2′-carboxamide (55) as a yellow solid (0.084 g, 44%) melting point 242-243° C. νₘₐₓ (ATR) 3384 (N—H), 2965 (N—H), 1654, 1642 (C═O), 1538, 1355 cm⁻¹. δ_{H} (400 MHz, CDCl₃) 12.87 (1H, s, 1-NH), 8.83 (1H, d, J 2.1, 6-H), 7.91 (1H, d, J 4.2, 4′—H), 7.68 (1H, d, J 4.2, 3′—H), 7.45 (1H, d, J 8.3, 3-H), 7.18 (1H, dd, J 8.3 J 2.1, 4-H), 6.37 (1H, s, NH—CH₃), 3.04 (3H, d, J 4.7, NH—CH₃), 1.36 (9H, s, C (CH₃)₃). δ_{C} (101 MHz, CDCl₃) 169.8 (2-C═O), 158.5 (2′-C═O), 157.5 (C-5), 154.7 (C-2′), 147.0 (C-5′), 139.5 (C-1), 128.7 (C-4′), 126.4 (C-3′), 126.3 (C-3), 121.0 (C-4), 118.6 (C-6), 116.6 (C-2), 35.5 (C(CH₃)₃), 31.1 (C(CH₃)₃), 27.1 (NH—CH₃). m/z (LCMS ES⁺) 362.3 [M+H]⁺. HRMS (ES⁺) found [M+H]⁺ 362.1179, C₁₇H₂₀N₃O₄S requires M 362.1175. CHN found: C 56.61; H 5.38; N 11.41. C₁₇H₁₉N₃O₄S requires C 56.49; H 5.30; N 11.63.

### N-{5-[2″-(2″-methoxyethoxy)ethoxy]-2-(methylcarbamoyl)phenyl}-5′-nitrothiophene-2′-carboxamide (51)

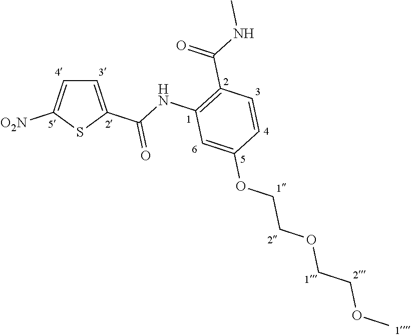 [SMILES: CNC(=O)c1ccc(OCCOCCOC)cc1NC(=O)c1ccc([N+](=O)[O-])s1]

Following general procedure Gii, using THE (1 mL) as a reaction solvent, where the substituted amine was 5-[2′-(2″-methoxyethoxy)ethoxy]-1-amino-N-methyl-2-benzamide (7.9 mg, 0.029 mmol, 1.0 eq.) afforded, without further purification, N-{5-[2″-(2″-methoxyethoxy)ethoxy]-2-(methylcarbamoyl)phenyl}-5′-nitrothiophene-2′-carboxamide (51) as a yellow solid (9.3 mg, 75%), melting point 204-205° C. νₘₐₓ (ATR) 3414 (N—H), 3119 (N—H), 1652 (C═O), 1586 (C═O), 1527, 1334 cm⁻¹. δ_{H} (599 MHz, CDCl₃) 13.16 (1H, s, 1-NH), 8.39 (1H, d, J 2.6, 6-H), 7.91 (1H, d, J 4.3, 4′—H), 7.68 (1H, d, J 4.3, 3′—H), 7.43 (1H, d, J 8.8, 3-H), 6.70 (1H, dd, J 8.8 J 2.6, 4-H), 6.29 (1H, s, NHCH₃), 4.21-4.25 (2H, m, 1″-H₂), 3.87-3.90 (2H, m, 2″-H₂), 3.71-3.74 (2H, m, 1″-H₂), 3.54-3.60 (2H, m, 2″-H₂), 3.39 (3H, s, 1″-H), 3.03 (3H, d, J 4.8, NHCH₃), δ_{C} (151 MHz, CDCl₃) 169.7 (2-C═O), 162.5 (C-5), 158.6 (2′-C═O), 154.8 (C-2′), 146.8 (C-5′), 141.7 (C-1), 128.6 (C-4′), 127.9 (C-3), 126.6 (C-3′), 111.7 (C-2), 111.2 (C-4), 105.9 (C-6), 72.1 (C-2″), 70.9 (C-1″), 69.6 (C-2″), 67.9 (C-1″), 59.2 (C-1″), 27.0 (NHCH₃). m/z (LCMS ES⁺) 424.4 [M+H]⁺. HRMS (ES⁺) found [M+H]⁺ 424.1194, C₁₈H₂₂N₃O₇S requires M 424.1178.

### N-[2-(methylcarbamoyl)-5-[2″-(morpholin-4″-yl)ethoxy]phenyl]-5′-nitrothiophene-2′-carboxamide (52)

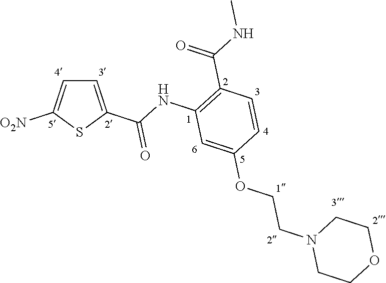 [SMILES: CNC(=O)c1ccc(OCCN2CCOCC2)cc1NC(=O)c1ccc([N+](=O)[O-])s1]

Following general procedure Gi, using THF (1 mL) as a reaction solvent, where the substituted amine was amino-N-methyl-5-[2′-(morpholin-4″-yl)ethoxy]-2-benzamide (248) (22 mg, 0.079 mmol, 1.0 eq.) afforded, after filtration and trituration with water of the crude precipitate and purification of the filtrate after work up by flash column chromatography (EtOAc:hexane), N-[2-(methylcarbamoyl)-5-[2″-(morpholin-4″-yl)ethoxy]phenyl]-5′-nitrothiophene-2′-carboxamide (52) as a yellow solid (10.7 mg, 31%), melting point 168-170° C. νₘₐₓ (ATR) 3428 (N—H), 1650 (C═O), 1536, 1331 cm⁻¹. δ_{H} (599 MHz, CDCl₃) 13.18 (1H, s, 1-NH), 8.40 (1H, d, J 2.6, 6-H), 7.91 (1H, d, J 4.3, 4′—H), 7.69 (1H, d, J 4.3, 3′—H), 7.43 (1H, d, J 8.8, 3-H), 6.69 (1H, dd, J 8.8 J 2.6, 4-H), 6.27 (1H, s, NHMe), 4.20 (2H, t, J 5.6, 1″—H₂), 3.71-3.76 (4H, m, 2″-H₂), 3.03 (3H, d, J 4.8, CH₃), 2.83 (2H, t, J 5.6, 2″—H₂), 2.59 (4H, J 4.8, 3″—H₂). δ_{C} (151 MHz, CDCl₃) 169.7 (2-C═O), 162.5 (C-5), 158.7 (2′-C═O), 154.9 (C-5′), 146.7 (C-2′), 141.7 (C-1), 128.6 (C-4′), 127.9 (C-3), 126.6 (C-3′), 111.7 (C-2), 111.2 (C-4), 105.9 (C-6), 67.1 (C-2″), 66.3 (C-1″), 57.6 (C-2″), 54.2 (C-3″), 27.0 (CH₃). m/z (LCMS ES⁺) 435.3 [M+H]⁺. HRMS (ES⁺) found [M+H]⁺ 435.1336, C₁₉H₂₃N₄O₆S requires M 435.1338.

### N-[2-(methylcarbamoyl)-5-[3″-(morpholin-4′″-yl) propoxy]phenyl]-5′-nitrothiophene-2′-carboxamide (53)

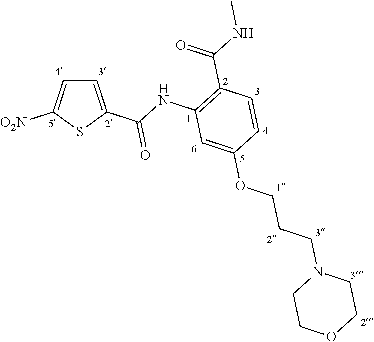 [SMILES: CNC(=O)c1ccc(OCCCN2CCOCC2)cc1NC(=O)c1ccc([N+](=O)[O-])s1]

Following general procedure Gi, using THF (3 mL) as a reaction solvent, where the substituted amine was amino-N-methyl-5-[3′-(morpholin-4″-yl) propoxy]-2-benzamide (0.100 g, 0.34 mmol, 1.0 eq.) afforded, without further purification, N-[2-(methylcarbamoyl)-5-[3″-(morpholin-4″-yl) propoxy]phenyl]-5′-nitrothiophene-2′-carboxamide (53) as a yellow solid (73 mg, 48%) melting point 206-208° C. νₘₐₓ (ATR) 3391 (N—H), 3100 (N—H), 1661 (C═O), 1641 (C═O), 1521, 1331 cm⁻¹. δ_{H} (599 MHz, CDCl₃) 13.19 (1H, s, 1-NH), 8.37 (1H, d, J 2.6, 6-H), 7.91 (1H, d, J 4.3, 4′—H), 7.69 (1H, d, J 4.3, 3′—H), 7.44 (1H, d, J 8.8, 3-H), 6.66 (1H, dd, J 8.8 J 2.6, 4-H), 6.31 (1H, s, NHMe), 4.13 (2H, t, J 6.3, 1″—H₂), 3.73-3.79 (4H, m, 2″-H₂), 3.02 (3H, d, J 4.8, CH₃), 2.46-2.62 (6H, m, 3″-H₂ & 3′″-H₂), 2.01-2.06 (2H, m, 2″-H₂). δ_{C} (151 MHz, CDCl₃) 169.7 (2-C═O), 162.7 (C-5), 158.7 (2′—C═O), 154.8 (C-5′), 146.8 (C-2′), 141.7 (C-1), 128.6 (C-4′), 127.9 (C-3), 126.6 (C-3′), 111.6 (C-2), 111.0 (C-4), 106.0 (C-6), 66.9 (C-2″), 66.5 (C-1″), 55.5 (C-3″), 53.8 (C-3″), 27.0 (CH₃), 26.1 (C-2″). m/z (LCMS ES⁺) 449.7 [M+H]⁺. HRMS (ES⁺) found [M+H]⁺ 449.1489, C₂₀H₂₅N₄O₆S requires M 449.1495.

### N-[2-(methylcarbamoyl)-5-[2″-(oxan-4″-yl)ethoxy]phenyl]-5′-nitrothiophene-2′-carboxamide (54)

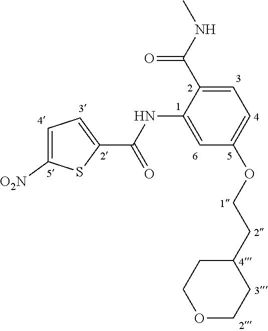 [SMILES: CNC(=O)c1ccc(OCCC2CCOCC2)cc1NC(=O)c1ccc([N+](=O)[O-])s1]

Following general procedure Gi, using THF (2 mL) as a reaction solvent, where the substituted amine was amino-N-methyl-5-[2′-(oxan-4″-yl))ethoxy]-2-benzamide (0.037 g, 0.13 mmol, 1.0 eq.) afforded, after purification by flash column chromatography (EtOAc:hexane), N-[2-(methylcarbamoyl)-5-[2″-(oxan-4″ yl)ethoxy]phenyl]-5′-nitrothiophene-2′-carboxamide (54) as a yellow solid (33 mg, 57%), melting point 144-146° C. νₘₐₓ (ATR) 3433 (N—H), 1638 (C═O), 1659 (C═O), 1537, 1333 cm⁻¹. δ_{H} (599 MHz, CDCl₃) 13.20 (1H, s, 1-NH), 8.39 (1H, d, J 2.6, 6-H), 7.91 (1H, d, J 4.3, 4′—H), 7.69 (1H, d, J 4.3, 3′—H), 7.43 (1H, d, J 8.8, 3-H), 6.66 (1H, dd, J 8.8 J 2.6, 4-H), 6.28 (1H, s, CH₃—NH), 4.11 (2H, t, J 6.1, 1″—H₂), 3.94-4.01 (2H, m, 2″-H₂), 3.41 (2H, td, J 11.9 J 2.1, 2″—H₂), 3.02 (3H, d, J 4.7, CH₃), 1.73-1.84 (3H, m, 2″-H₂ & 4″-H), 1.64-1.69 (2H, m, 3″-H₂), 1.32-1.42 (2H, m, 3″-H₂). δ_{C} (151 MHz, CDCl₃) 169.7 (2-C═O), 162.8 (C-5), 158.7 (2′-C═O), 154.8 (C-5′), 146.8 (C-2′) 141.8 (C-1), 128.6 (C-4′), 127.9 (C-3), 126.6 (C-3′), 111.5 (C-2), 111.1 (C-4), 105.8 (C-6), 68.1 (C-2″), 65.8 (C-1″), 36.1 (C-2″), 33.1 (C-3″), 32.0 (C-4″), 27.0 (CH₃). m/z (LCMS ES⁺) 434.6 [M+H]⁺. HRMS (ES⁺) found [M+H]⁺ 434.1388, C₂₀H₂₄N₃O₆S requires M 434.1386.

### Biological Experimental

All biological results are reported as the mean of three biological repeats, with errors representing standard error in each case.

### General Reagents

Biological grade reagents, buffers and media were commercially sourced. Media and buffers were made up using purified water from a MilliQ® water purification system. Biorelevant buffers (FESSIF, FASSIF and FASSGF) were purchased from Biorelevant and made up according to manufacturer's instructions. PBS tablets were obtained from Fischer Scientific.

Schneider's media throughout refers to Schneiders insect media (Merck) supplemented with 15% fetal bovine serum (FBS, heat inactivated, South American origin, ThermoFisher scientific) and 1% Penicillin-Streptomycin (PenStrep, Gibco™, 10,000 U mL⁻¹ ThermoFisher scientific). DMEM refers to Dulbecco's Modified Eagle Medium (Gibco™ DMEM, high glucose, GlutaMAX™ Supplement, pyruvate, ThermoFisher scientific) supplemented with 10% FBS and 1% PenStrep. Schneider's media and DMEM were filter sterilised using sterile vacuum filter flasks (0.22 μm pore CA membrane, Merck) and stored at 4° C.

### Chemicals

Clemastine fumarate, tamoxifen, resazurin, daidzin, miltefosine, amphotericin B and cycloheximide were purchased from Merck (formerly Sigma Aldrich). Rhodamine azide and biotin azide were synthesised in house by Dr Exequiel Porta. All synthesised compounds and controls were dissolved in biological grade DMSO (Fisher Bioreagents) to give 10 mM stocks unless otherwise stated.

### General Equipment

Centrifuge tubes, serological pipettes, microcentrifuge tubes, reagent reservoirs and pipette tips were obtained from Fisher Scientific or StarLab and autoclaved if non-sterile (121° C., 5 mins). Centrifugation steps were carried out using a Beckman Coulter centrifuge, Simga1-14 microfuge, Sorvall® Legend RT centrifuge, Sorvall® Legend Micro 17R centrifuge or Boeco U-320. Vortexing was performed using a Vortex Mixer MT20 Chiltern®.

### Cell Lines

All parasite and human cell cultures were grown and manipulated following biosafety level 2 instructions under sterile conditions. L. major FV1, L. amazonensis (MHOM/Br/75/JOSEFA), L. donovani BOB (derived from MHOM/SD/62/1S-CL2D), T. brucei (90-13, 427 strain) and T. cruzi (Dm28c) were used throughout.

### Buffers and Media

Buffers and media were prepared as follows and made up to the specified volume using Milli-Q grade purified water. Where necessary, pH was adjusted using concentrated HCl, saturated sodium hydroxide solution, or dilutions thereof.

TABLE 9
Compositions of buffers used throughout. *Stable
for weeks-months if stored at −80° C.

| | Final | | | | |
|---|---|---|---|---|---|
| | volume/ | | | | Final |
| Buffer | mL | Stock | Quantity | | concentration |
| Resazurin | 40 | Resazurin sodium | 5 | mg | 500 μM |
| solution | | salt PBS | 40 | mL | |

TABLE 10
Composition of media used throughout. *Ampicillin is added
following autoclave sterilisation or microwaving.

| | Final | | | |
|---|---|---|---|---|
| Media | volume/mL | Stock solution | Quantity | |
| Schneider's | 1000 | Schneider's Insect Medium | 24.5 | g |
| Insect | | NaHCO₃ | 0.4 | g |
| Medium | | CaCl₂ | 0.6 | g |
| LIT media | 1000 | Liver infusion | 5.0 | g |
| | | Tryptose | 5.0 | g |
| | | NaCl | 4.0 | g |
| | | Na₂HPO₄ | 8.0 | g |
| | | KCl | 0.40 | g |
| | | Glucose | 8.0 | g |

### Biological Procedures

### Cell Culture and Assays

### Leishmania Promastigote Cell Culture

All steps were carried out under sterile conditions. Frozen stocks of the relevant Leishmania promastigote species were thawed rapidly and added to Schneider's media. Promastigotes were grown at 26° C. in Nunc EasYFlask 25 cm² Nunclon Delta Surface (ThermoScientific) and frozen stocks were prepared on low passage cultures (p2-4) by pelleting the cells by centrifugation and resuspending in Schneider's media with 10% DMSO. The cultures were aliquoted into cryovials (Starlab) and cooled slowly to −150° C. for long term storage.

T. cruzi Cell Culture

All steps were carried out under sterile conditions. Epimastigotes were grown at 28° C. and maintained at exponential phase by dilution every 2-3 days with fresh LIT media supplemented with 10% FBS and 5 μM hemin.

### HepG2 Cell Culture

All steps were carried out under sterile conditions. HepG2 cells were thawed rapidly into DMEM. Cells were grown at 37° C., 5% CO₂ for >5 days in T-75 CytoOne® Flask, TC-Treated, Vented flasks (Starlab) in a Sanyo MCO-18M incubator. HepG2 cells were passaged by decanting media, washing with 3×5 mL pre-warmed sterile PBS and disrupting with pre-warmed Gibco™ TrypLE solution (Fisher Scientific) for 10 mins at 37° C. The trypsin was deactivated by addition of DMEM. The cells were homogenised and 5 mL of cell culture was added to 20 mL fresh DMEM. Cells were split 1:4 every 3 days. Frozen stocks were prepared in the same way as the passage, but resuspended into DMEM with 10% DMSO, aliquoted into cryovials (Starlab) and cooled slowly to −150° C. for long term storage.

### Dose Response Assays

L. major and L. amazonensis Promastigote

All steps were carried out under sterile conditions. Leishmania promastigotes were incubated (1×10⁶ parasites mL⁻¹ for L. major and 0.5×10⁶ parasites mL⁻¹ for L. amazonensis) with a 3× serial dilution of compound in Schneider's media in Corning™ Costar™ 96-Well Flat-Bottom Microplates (Fisher Scientific) at 26° C. for 44 hrs. Cycloheximide or amphotericin B were used as positive controls and 2% DMSO was used as a negative control. alamarBlue® (ThermoFisher scientific) or resazurin solution was added to each well and incubated for a further 4 hrs at 26° C. Cell-viability was determined using a BioTek™ FLx800 microplate reader with Gen5® 1.08 data analysis software (BioTek) by monitoring fluorescence emission (λₑₓ 560/λₑₘ 590 nm). Dose response curves were fitted using sigmoidal regression analysis (GraphPad Prism 8 software) and used to calculate EC₅₀ values.

L. donovani Promastigote

All steps were carried out under sterile conditions by Dr Juliana Pacheco, Wellcome centre for Anti-Infectives Research, Dundee. L. donovani promastigotes (1×10⁵ parasites mL⁻¹) were incubated with a serial dilution of compound in modified M199 media for 72 hrs at 26° C. Resazurin (50 μM) was added to each well and incubated for a further 3 hrs at 26° C. Cell-viability was determined using a fluorescence plate reader λₑₓ 528/λₑₘ 590 nm. Dose response curves were fitted using GRAFIT (version 5.0.4, Erithacus software) and used to calculate EC₅₀ values.

T. brucei Trypomastigotes

All steps were carried out under sterile conditions by members of the Professor Jim Morris lab, Clemson, South Carolina. T. brucei bloodstream trypomastigotes (1×10⁵ cells mL⁻¹) were incubated with a serial dilution of compound in HMI-9 (10% FBS, 10% NuSerum) in black 384-well polystyrene plates for 48 hrs (37° C., 5% CO₂). CellTiter Blue was added, and the plate incubated for 1 hr before removing the plate lid to allow pH to equilibrate across the plate and recording the fluorescence (Nex 546/λₑₘ: 585 nm). Dose response curves were fitted using sigmoidal regression analysis (GraphPad Prism 8 software) and used to calculate EC₅₀ values.

T. cruzi Epimastigotes

All steps were carried out under sterile conditions by members of the Guille Labadie lab, National university of Rosario, Argentina. T. cruzi epimastigotes (2×10⁶ cells mL⁻¹) were incubated with a serial dilution of compound in LIT (10% FBS, 5 μM hemin) in a sealed 96-well plate at 28° C. for 72 hours. Epimastigotes were fixed with formaldehyde (final concentration 4%) and counted. Dose response curves were fitted using sigmoidal regression analysis (GraphPad Prism 8 software) and used to calculate EC₅₀ values.

### HepG2

All steps were carried out under sterile conditions. 80-100% confluent HepG2 were plated into Corning™ Costar™ 96-Well Flat-Bottom Microplates (Fisher Scientific) by adding 200 μL of a homogenous dispersion of cells at a concentration of 0.6×10⁵ cells mL⁻¹ before incubation at 37° C., 5% CO₂ overnight. The media was then removed and compounds, diluted in DMEM (10% FBS, 1% PenStrep) were added to the prepared plates using a 3× serial dilution from 100 μM (DMSO<2%) to give a final well volume of 100 μL (see footnote¹). The plates were incubated at 37° C., 5% CO₂ for 44 hrs in a Sanyo MCO-18M incubator. alamarBlue® (ThermoFisher scientific) or resazurin solution (10 μL) was added to each well and incubated for a further 4 hrs at 37° C., 5% CO₂. Cell-viability was determined using a BioTek™ FLx800 microplate reader with Gen5® 1.08 data analysis software (BioTek) by monitoring fluorescence emission (Nex 560/hem 590 nm). Dose response curves were fitted using sigmoidal regression analysis (GraphPad Prism 8 software) and used to calculate EC₅₀ values.

### DMPK Studies

DMPK studies were performed by GVK BIO Sciences Pvt Ltd Hyderabad, India, in collaboration with LifeArc. Three swiss albino mice per experiment were inoculated (IP or oral) with compound at 10 mg kg⁻¹ in 10% DMSO (2 mg mL⁻¹) on day 1. A small blood sample (50 μL) was taken after 15 minutes, 30 minutes, 1 hr, 2 hr, 4 hr, 6 hr, 8 hr and 24 hr. Proteins were precipitated in acetonitrile and the supernatant was diluted in 1:1 methanol:water and subject to LCMS analysis. The concentration of compound in the blood was determined by comparing the peak area to a calibration curve.

### Preparation of Liposome Compositions

Liposomal compositions were prepared by incorporation of an active compound into the lipid bilayer of SA-bearing choline liposome (lipid ratio, 7:2). In brief, liposomes were prepared by adding 285 μg of the drug to the lipids, 20 mg phosphotidylcholine (PC, Sigma) and 2 mg stearylamine (SA, Fluka, Switzerland) to form a clear solution in chloroform-methanol, followed by evaporating the organic solvents to form a thin film in a round-bottom flask. The film was dried overnight in a vacuum dessicator. The film was rehydrated in 1 ml (for in-vitro) and 285 μl (for in-vivo) of 20 mM PBS (pH 7.4), and the suspension was sonicated for 30 s (once for in-vitro and twice for in-vivo) in an ultrasonicator, followed by incubation for 2 h at 4° C. before use. The liposomes were stored at 4° C. and the activity remains intact at least till 1 month.

### Antibacterial Screening Methods

Technical and biological MIC assays were carried out using S. aureus (FDA209P) or E. coli (BW25113 or ΔrfaC) in cation-supplemented MHB using 50 μl of serially diluted compound, and 50 μl of bacterial culture. DMSO was used as a solvent control at a maximum final concentration of 10% (a greater percentage than would be achieved using the compounds at the concentrations in question). Plates were incubated at 37° C. with shaking for 16 hours before measuring absorbance at 600 nm. These values were then used to calculate percentage growth at each dilution of compound in comparison to growth in the absence of compound. The following day, the contents of each well were resuspended using a multichannel pipette, and 5 μl from each well was then applied to LB agar plates. These were incubated at 30° C. in a stationary incubator for 16 hours before imaging.

### Physiochemical Experimental

### Materials and Methods

All chemicals and solvents were sourced from commercial suppliers. Buffers were made up using purified water from a MilliQ® water purification system. Biorelevant buffers (FESSIF, FASSIF and FASSGF) were purchased from Biorelevant and made up according to manufacturer's instructions. 15% FBS (heat inactivated, South American origin, ThermoFisher scientific) in PBS (Fisher Scientific) was filter sterilised (0.22 μm Fisherbrand™ Sterile PES Syringe Filter, Fisher Scientific) before use. Sodium taurocholate was purchased from Merck.

### Procedures

### Kinetic Solubility

λₘₐₓ was determined for each compound from the UV-Vis absorption spectrum of a 200 LIM compound solution in buffer of interest using a Varian Cary® 100 UV-Vis Spectrophotometer. A calibration curve of λₘₐₓ VS concentration was obtained by serial dilution of compound from 200 μM in the buffer of interest, correcting for background buffer absorption. For each sample, a DMSO solution of compound was incubated in buffer of interest (<5% DMSO) for 2 hrs at RT. The solution was filtered (0.22 μm Fisherbrand™ Sterile PES Syringe Filter, Fisher Scientific) or centrifuged (14,500 rpm, 10 min) to remove undissolved debris and the supernatant was removed and diluted as required. The corrected UV absorption of the supernatant at Amax Was measured using a Varian Cary® 100 UV-Vis Spectrophotometer and compared to the calibration curve to determine compound solubility.

### REFERENCES

(1) Torpiano, P.; Pace, D. Leishmaniasis: Diagnostic Issues in Europe. Expert Rev. Anti. Infect. Ther. 2015, 13 (9), 1123-1138. https://doi.org/10.1586/14787210.2015.1056160.
(2) Norcliffe, J. L.; Mina, J. G.; Alvarez, E.; Cantizani, J.; de Dios-Anton, F.; Colmenarejo, G.; Valle, S. G.-D.; Marco, M.; Fiandor, J. M.; Martin, J. J.; Steel, P. G.; Denny, P. W. Identifying Inhibitors of the Leishmania Inositol Phosphorylceramide Synthase with Antiprotozoal Activity Using a Yeast-Based Assay and Ultra-High Throughput Screening Platform. Nat. Sci. reports 2018, 8 (3938), 1-10. https://doi.org/10.1038/s41598-018-22063-9.
(3) Zhang, H.; Collins, J.; Nyamwihura, R.; Crown, O.; Ajayi, O.; Ogungbe, I. V. Vinyl Sulfone-Based Inhibitors of Trypanosomal Cysteine Protease Rhodesain with Improved Antitrypanosomal Activities. Bioorganic Med. Chem. Lett. 2020, 30 (14), 127217. https://doi.org/10.1016/j.bmcl.2020.127217.
(4) Pena, I.; Manzano, M. P.; Cantizani, J.; Kessler, A.; Alonso-padilla, J.; Bardera, A. I.; Alvarez, E.; Colmenarejo, G.; Cotillo, I.; Roquero, I.; Dios-anton, F. De; Barroso, V.; Rodriguez, A.; Gray, D. W.; Navarro, M.; Kumar, V.; Sherstnev, A.; Drewry, D. H.; Brown, J. R.; Fiandor, J. M.; Martin, J. J. New Compound Sets Identified from High Throughput Phenotypic Screening Against Three Kinetoplastid Parasites: An Open Resource. Nat. Sci. reports 2015, 5 (8871), 1-12.
(5) Lamotte, S.; Aulner, N.; Späth, G. F. Discovery of Novel Hit Compounds with Broad (5) Activity against Visceral and Cutaneous Leishmania Species by Comparative Phenotypic Screening. Nat. Sci. Reports 2019, 9 (438), 1-11. https://doi.org/10.1038/s41598-018-36944-6.
(6) Hefnawy, A.; Cantizani, J.; Peña, I.; Manzano, P.; Rijal, S.; Dujardin, J.-C.; De Muylder, G.; Martin, J. Importance of Secondary Screening with Clinical Isolates for Anti-Leishmania Drug Discovery. Sci. Rep. 2018, 8 (1), 11765. https://doi.org/10.1038/s41598-018-30040-5.
(7) Khare, S.; Nagle, A. S.; Biggart, A.; Lai, Y. H.; Liang, F.; Davis, L. C.; Barnes, S. W.; Mathison, C. J. N.; Myburgh, E.; Gao, M. Y.; Gillespie, J. R.; Liu, X.; Tan, J. L.; Stinson, M.; Rivera, I. C.; Ballard, J.; Yeh, V.; Groessl, T.; Federe, G.; Koh, H. X. Y.; Venable, J. D.; Bursulaya, B.; Shapiro, M.; Mishra, P. K.; Spraggon, G.; Brock, A.; Mottram, J. C.; Buckner, F. S.; Rao, S. P. S.; Wen, B. G.; Walker, J. R.; Tuntland, T.; Molteni, V.; Glynne, R. J.; Supek, F. Proteasome Inhibition for Treatment of Leishmaniasis, Chagas Disease and Sleeping Sickness. Nature 2016, 537 (7619), 229-233. https://doi.org/10.1038/nature19339.
(8) Hackler, A.; Patrick, S. L.; Kahney, E. W.; Flaherty, D. P.; Sharlow, E. R.; Morris, J. C.; Golden, J. E. Antiparasitic Lethality of Sulfonamidebenzamides in Kinetoplastids. Bioorganic Med. Chem. Lett. 2017, 27 (4), 755-758. https://doi.org/10.1016/j.bmcl.2017.01.043.
(9) Flaherty, D. P.; Miller, J. R.; Garshott, D. M.; Hedrick, M.; Gosalia, P.; Li, Y.; Milewski, M.; Sugarman, E.; Vasile, S.; Salaniwal, S.; Su, Y.; Smith, L. H.; Chung, T. D. Y.; Pinkerton, A. B.; Aube, J.; Callaghan, M. U.; Golden, J. E.; Fribley, A. M.; Kaufman, R. J. Discovery of Sulfonamidebenzamides as Selective Apoptotic CHOP Pathway Activators of the Unfolded Protein Response. ACS Med. Chem. Lett. 2014, 5 (12), 1278-1283. https://doi.org/10.1021/ml5003234.
(10) Zhang, H.; Collins, J.; Nyamwihura, R.; Ware, S.; Kaiser, M.; Ogungbe, I. V. Discovery of a Quinoline-Based Phenyl Sulfone Derivative as an Antitrypanosomal Agent. Bioorganic Med. Chem. Lett. 2018, 28 (9), 1647-1651. https://doi.org/10.1016/j.bmcl.2018.03.039.
(11) Gewald, K. Methods for the Synthesis of 2-Aminothiophenes and Their Reactions (Review). Chem. Heterocycl. Compd. 1977, 12 (10), 1077-1090. https://doi.org/10.1007/BF00945583.
(12) Galvez, C.; Garcia, F. Functional Derivatives of Thiophene 1. Synthesis and 1HNMR Spectra in the 2-Aminothiophene Series. J. Heterocycl. Chem. 1981, 18, 851-853.
(13) Rashmi, P.; Nargund, L. V. G.; Hazra, K.; Chandra, J. N. N. S. Thienopyrimidines as Novel Inhibitors of Mycobacterium Tuberclosis: Synthesis and in-Vitro Studies. Arch. Pharm. Chem. Life Sci. 2011, 344 (7), 459-465. https://doi.org/10.1002/ardp.201000394.
(14) Bugge, S.; Kaspersen, S. J.; Sundby, E.; Hoff, B. H. Route Selection in the Synthesis of C-4 and C-6 Substituted Thienopyrimidines. Tetrahedron 2012, 68 (45), 9226-9233. https://doi.org/10.1016/j.tet.2012.08.090.
(15) Jasial. S.; Hu, Y.; Bajorath, J. How Frequently Are Pan-Assay Interference Compounds Active? Large-Scale Analysis of Screening Data Reveals Diverse Activity Profiles, Low Global Hit Frequency, and Many Consistently Inactive Compounds. J. Med. Chem. 2017, 60 (9). 3879-3886. https://doi.org/10.1021/acs.jmedchem.7b00154.

1. A compound of formula (I): 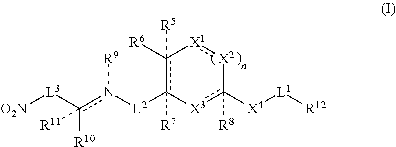
wherein X¹ is CR¹, C(R¹)₂, N, NR¹, O or S;
X² is CR², C(R²)₂, N, NR², O or S;
X³ is CR³, C(R³)₂, N, NR³, O or S;
X⁴ is a bond or NR⁴, O or S;
L¹ is optionally substituted C₁₋₁₂ alkylene, optionally substituted C₂₋₁₂ alkenylene, optionally substituted C₂₋₁₂ alkynylene or -(L₄O)ₘL⁵-;
L² is a bond or is optionally substituted C₁₋₁₂ alkylene, optionally substituted C₂₋₁₂ alkenylene, optionally substituted C₂₋₁₂ alkynylene or -(L₄O)ₘL⁵-;
L³ is an optionally substituted 5 to 10 membered heteroarylene or an optionally substituted C₆₋₁₀ arylene;
L⁴ is optionally substituted C₁₋₆ alkylene, optionally substituted C₂₋₆ alkenylene or optionally substituted C₂₋₆ alkynylene;
L⁵ is a bond or optionally substituted C₁₋₆ alkylene, optionally substituted C₂₋₆ alkenylene or optionally substituted C₂₋₆ alkynylene;
R¹, R² and R³ are each independently H, optionally substituted C₁₋₁₂ alkyl, optionally substituted C₂₋₁₂ alkynyl, optionally substituted C₂₋₁₂ alkenyl, OR¹³, SR¹³, NR¹³R¹⁴, COR¹³, COOR¹³, CONR¹³R¹⁴, CN or a halogen;
R⁴ is H, optionally substituted C₁₋₁₂ alkyl, optionally substituted C₂₋₁₂ alkenyl or optionally substituted C₂₋₁₂ alkynyl;
R⁵, R⁷ and R⁸ are independently absent or H, optionally substituted C₁₋₁₂ alkyl, optionally substituted C₂₋₁₂ alkynyl, optionally substituted C₂₋₁₂ alkenyl, OR¹³, SR¹³, NR¹³R¹⁴, COR¹³, COOR¹³, CONR¹³R¹⁴, CN or a halogen;
R⁶ is H, optionally substituted C₁₋₁₂ alkyl, optionally substituted C₂₋₁₂ alkynyl, optionally substituted C₂₋₁₂ alkenyl, OR¹³, SR¹³, NR¹³R¹⁴, COR¹³, COOR¹³, CONR¹³R¹⁴, CN or a halogen;
R⁹ is absent or is H, optionally substituted C₁₋₁₂ alkyl, optionally substituted C₂₋₁₂ alkenyl or optionally substituted C₂₋₁₂ alkynyl; or R⁶ and R⁹ together with the atoms to which they are attached combine to form an optionally substituted 5 or 6 membered heterocycle or an optionally substituted 5 or 6 membered heteroaryl;
R¹⁰ is H, halogen, optionally substituted C₁₋₁₂ alkyl, optionally substituted C₂₋₁₂ alkenyl or optionally substituted C₂₋₁₂ alkenyl; R¹¹ is absent or H, halogen, optionally substituted C₁₋₁₂ alkyl, optionally substituted C₂₋₁₂ alkenyl or optionally substituted C₂₋₁₂ alkenyl; or R⁶ and R¹⁰ together with the atoms to which they are attached combine to form an optionally substituted 5 or 6 membered heterocycle or an optionally substituted 5 or 6 membered heteroaryl; or R¹⁰ and R¹¹ together form an oxo group;
R¹² is NR¹³R¹⁴, an optionally substituted 5 to 10 membered heteroaryl or an optionally substituted 3 to 10 membered heterocycle, where the heteroaryl or heterocycle contain at least one nitrogen;
R¹³ and R¹⁴ are independently H, optionally substituted C₁₋₁₂ alkyl, optionally substituted C₂₋₁₂ alkenyl or optionally substituted C₂₋₁₂ alkynyl;
n is 0 or 1; and
m is an integer between 1 and 5;
or a pharmaceutically acceptable complex, salt, solvate, tautomeric form or polymorphic form thereof.

2. The compound of claim 1, wherein the compound is a compound of formula (Ib):

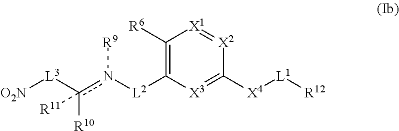

3. The compound of claim 2, wherein the compound is a compound of formula (Ibi):

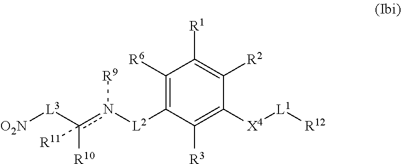

4. The compound of claim 1, wherein n is 0, optionally wherein X¹ is CR¹ and R⁵, R⁷ and R⁸ are absent.

5. (canceled)

6. The compound according to claim 1, wherein at least one of R¹, R², R³, R⁵, R⁶, R⁷ and R⁸ is optionally substituted C₁₋₁₂ alkyl, optionally substituted C₂₋₁₂ alkynyl, optionally substituted C₂₋₁₂ alkenyl, OR¹³, SR¹³, NR¹³R¹⁴, COR¹³, COOR¹³, CONR¹³R¹⁴, CN or a halogen, optionally wherein at least one of R¹ and R⁶ is optionally substituted C₁₋₁₂ alkyl, optionally substituted C₂₋₁₂ alkynyl, optionally substituted C₂₋₁₂ alkenyl, OR¹³, SR¹³, NR¹³R¹⁴, COR¹³, COOR¹³, CONR¹³R¹⁴, CN or a halogen.

7. (canceled)

8. The compound according to claim 6, wherein one of R¹ and R⁶ is COOR¹³ or CONR¹³R¹⁴, and R¹³ and R¹⁴ are independently H, optionally substituted C₁₋₆ alkyl, optionally substituted C₂₋₆ alkenyl or optionally substituted C₂₋₆ alkynyl, optionally wherein R⁶ is CONHMe.

9. (canceled)

10. The compound according to claim 1, wherein R¹, R² and R³ are H.

11. The compound according to claim 1, wherein X⁴ is O.

12. The compound according to claim 1, wherein L¹ is optionally substituted C₁₋₆ alkylene, optionally substituted C₂₋₆ alkenylene, optionally substituted C₂₋₆ alkynylene or -(L₄O)ₘL⁵-, and L⁴ and L⁵ are optionally substituted C₁₋₃ alkylene, optionally substituted C₂₋₃ alkenylene or optionally substituted C₂₋₃ alkynylene and m is 1, 2 or 3, preferably wherein L¹ is optionally substituted C₂₋₃ alkylene, optionally substituted C₂₋₃ alkenylene or optionally substituted C₂₋₃ alkynylene.

13. The compound according to claim 1, wherein R¹² is NR¹³R¹⁴, an optionally substituted 5 or 6 membered heteroaryl or an optionally substituted 5 or 6 membered heterocycle, where the heteroaryl or heterocycle contain at least one nitrogen, and R¹³ and R¹⁴ are independently H, optionally substituted C₁₋₆ alkyl, optionally substituted C₂₋₆ alkenyl or optionally substituted C₂₋₆ alkynyl, optionally wherein R¹² is an optionally substituted 5 or 6 membered heterocycle, where the heterocycle contains at least one nitrogen.

14. (canceled)

15. The compound according to claim 1, wherein L² is a bond or optionally substituted C₁₋₆ alkylene, optionally substituted C₂₋₆ alkenylene or optionally substituted C₂₋₆ alkynylene, and preferably wherein L² is a bond.

16. The compound according to claim 1, wherein R⁹ is H, optionally substituted C₁₋₆ alkyl, optionally substituted C₂₋₆ alkenyl or optionally substituted C₂₋₆ alkynyl, and preferably is H.

17. The compound according to claim 1, wherein R¹⁰ and R₁₁ together form an oxo group.

18. The compound according to claim 1, wherein L³ is an optionally substituted 5, 6 or 9 membered heteroarylene or an optionally substituted phenylene, and preferably is

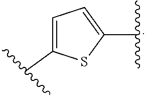 [SMILES: *c1ccc(*)s1]

19. The compound according to claim 1, wherein the compound is a compound of formula (100) or (101):

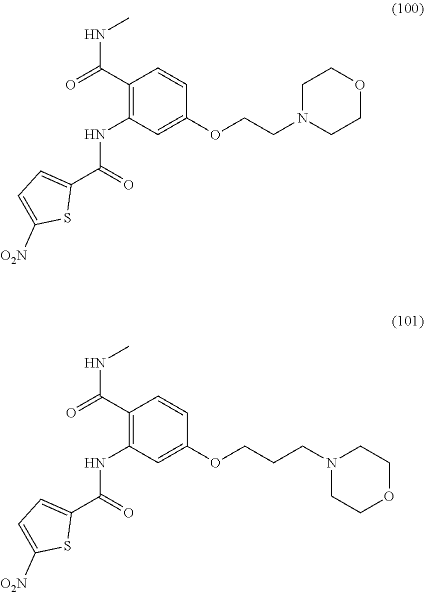 [SMILES: CNC(=O)c1ccc(OCCN2CCOCC2)cc1NC(=O)c1ccc([N+](=O)[O-])s1 ; CNC(=O)c1ccc(OCCCN2CCOCC2)cc1NC(=O)c1ccc([N+](=O)[O-])s1]

20. A pharmaceutical composition comprising a compound of formula (I), as defined by claim 1, or a pharmaceutically acceptable complex, salt, solvate, tautomeric form or polymorphic form thereof, and a pharmaceutically acceptable vehicle.

21. The pharmaceutical composition according to claim 20, wherein the composition is a liposomal suspension or formulation comprising a plurality of lipids.

22. (canceled)

23. A method of treating, preventing or ameliorating an infection in a subject, the method comprising administering to a subject in need of such treatment, a therapeutically effective amount of a compound of formula (I), as defined by claim 1, or a pharmaceutically acceptable complex, salt, solvate, tautomeric form or polymorphic form thereof.

24. The method according to claim 23, wherein the infection is a parasitic infection, and preferably is leishmaniasis, Chagas disease or African sleeping sickness, optionally wherein the parasitic infection is leishmaniasis.

25. (canceled)

26. The compound or pharmaceutical composition for use according to claim 23, wherein the infection is a bacterial infection.
